(19) United States
(12) Patent Application Publication
(10) Pub. No.: US 2025/0170254 A1
(43) Pub. Date: May 29, 2025

(54) Subdermal Implant for Sustained Drug Delivery

(71) Applicant: OAK CREST INSTITUTE OF SCIENCE, Monrovia, CA (US)

(72) Inventors: Marc M. Baum, Altadena, CA (US); Manjula Gunawardana, Claremont, CA (US)

(21) Appl. No.: 18/725,371

(22) PCT Filed: Jan. 6, 2023
(86) PCT No.: PCT/US2023/060248

§ 371 Date: Jun. 28, 2024

Related U.S. Application Data
(60) Provisional application No. 63/297,191, filed on Jan. 6, 2022.

(51) Int. Cl. A61K 47/64; A61K 9/00; A61K 47/32; A61K 47/54

(52) U.S. Cl. CPC A61K 47/645; A61K 9/0024; A61K 47/32; A61K 47/542

(57) ABSTRACT

This disclosure relates to the use of a long-acting implantable or injectable device to deliver biologically active compounds, e.g., antiviral compounds such as peptide conjugates of antiviral drugs disclosed herein, at a controlled rate for an extended period of time and methods of manufactures thereof. The device is biocompatible and biostable, and is useful as an implant and/or injectable in patients (humans and animals) for the delivery of appropriate bioactive substances to tissues or organs.

### CROSS-REFERENCE TO RELATED APPLICATIONS

Priority is claimed to U.S. Ser. No. 63/297,191, filed Jan. 6, 2022, and the disclosures thereof are hereby incorporated by reference in their entirety.

### STATEMENT OF GOVERNMENT INTEREST

This invention was made with government support under AI120748, R01 HD101344, U19AI113048, and R01AI154561 awarded by the National Institutes of Health (NIH). The government has certain rights in the invention.

### FIELD OF INVENTION

This disclosure generally relates to the field of implantable sustained release drug delivery devices.

### BACKGROUND

Drug delivery is an important area of medical treatment. The efficacy of many drugs is directly related to how they are administered. Present modes of drug delivery such as topical application, oral delivery, as well as intramuscular, intravenous, and subcutaneous injection may result in high and low blood concentrations and/or shortened half-life in the blood. In some cases, achieving therapeutic efficacy with these standard administrations requires large doses of medications that may result in toxic side effects. The technologies relating to controlled drug release have been attempted in an effort to circumvent some of the pitfalls of conventional therapy. Their aims are to deliver medications in a continuous and sustained manner. Additionally, local controlled drug release applications are site or organ specific (e.g., controlled intravaginal delivery) and can minimize systemic exposure to the agent.

Traditional routes of administration are problematic in that they require strict patient compliance; i.e., when medication is administered orally, such as an antibiotic, hormone, vitamin, or when repeated visits to the doctor are necessary because the route of administration is by injection. These methods of administration are especially problematic in cases where the patient is a child, is elderly, or where the medication must be administered on a chronic basis; i.e., weekly allergy injections. Compliance with taking medication is a problem for many adults, as they simply forget to take it. Further, weekly injections deter many people from obtaining needed treatment because weekly injections at the doctor's office interferes with their activities or schedules. In other words, adherence to frequent dosing is burdensome to the user and has emerged as a key factor in explaining the heterogeneous efficacy outcomes of many therapeutic and prophylactic regimens. Sustained release or “long-acting” drug formulations hold significant promise as a means of reducing dosing frequency, thereby increasing the effectiveness of the regimen.

Implantable microdevice, reservoir delivery systems do not require user intervention and, therefore, overcome the above adherence concerns. In recent years, the development of microdevices for local drug delivery is one area that has proceeded steadily. Activation of drug release can be passively or actively controlled. They are theoretically capable of delivering the drug for months, possibly even years, at a controlled rate and are often comprised of a polymeric material. Implants of polymeric material as drug delivery systems have been known for some time. Implantable delivery systems of polymeric material are known for instance for the delivery of contraceptive agents, either as subcutaneous implants or intravaginal rings. Prior art implants do not sufficiently control drug release. Various devices have been proposed for solving this problem. However, none have been entirely satisfactory. Such problems result in a drug delivery device that administers drugs in an unpredictable pattern, thereby resulting in poor or reduced therapeutic benefit.

Injectable, long-acting delivery systems also are known in the art to be beneficial. These include: particle suspensions that are injected subcutaneously, intramuscularly, or to other anatomic compartments; formulations that solidify into solid or semisolid implants after injection into an anatomic compartment such as the subcutaneous space; and microarray (microneedle) patches, consisting of microscopic projections applied to skin's outermost layer to deliver their cargo to the epidermal/dermal layer.

For example, a popular drug delivery device is a drug eluting stent. Stents are mesh-like steel or plastic tubes that are used to open a clogged atherosclerotic coronary artery or a blood vessel undergoing stenosis. A drug may be attached onto, or impregnated into, the stent that is believed to prevent re-clogging or restenosis a blood vessel. However, the initial release of the drug may be very rapid releasing 20-40% of the total drug loading in a single day. Such high concentrations of the drug have been reported to result in cytotoxicity at the targeted site. As a result of these problems, there is a need for a drug delivery device that can be optimized to deliver any therapeutic, diagnostic, or prophylactic agent for any time period up to several years maintaining a controlled and desired rate.

Microdevices implanted in various anatomic sites can be divided roughly into two categories: resorbable polymer-based devices and nonresorbable devices. Polymer devices have the potential for being biodegradable, therefore avoiding the need for removal after implantation.

Non-biodegradable drug delivery systems include, for example, Vitrasert® (Bausch & Lomb, Inc.), a surgical implant that delivers ganciclovir intraocularly; Duros® (Alza Corp.), surgically implanted osmotic pump that delivers leuprolide acetate to treat advanced prostate cancer; and Implanon™ (Merck & Co., Inc.), a type of subdermal contraceptive implant. Additionally, there exist commercial implant devices that are used vaginally, such as NuvaRing® (Merck & Co., Inc.), an intravaginal ring that delivers etonogestrel and ethinyl estradiol for contraception.

Biodegradable implants include, for example, Lupron Depot® (leuprolide acetate, TAP Pharm. Prods., Inc.), a sustained-release microsphere-suspension injection of luteinizing hormone-releasing hormone (LH-RH) analog for the treatment of prostate cancer; and the Posurdex® dexamethasone anterior segment drug delivery system (Allergan, Inc.).

Parenteral, long-acting drug delivery systems, both systemic and local, rely on favorable drug pharmacologic properties to be feasible. The dosing frequency of long-acting drug candidates is determined by the drug's aqueous solubility, potency, and systemic clearance kinetics, among other characteristics. These criteria severely limit the number of suitable agents for development. The field of HIV-1 prevention and treatment exemplifies this predicament, and is discussed as non-limiting, illustrative examples below.

A long-acting injectable formulation of the integrase strand transfer inhibitor (ISTI) cabotegravir (CAB) has demonstrated superiority over daily oral tenofovir disoproxil fumarate (TDF)-emtricitabine (FTC) in the prevention of HIV-1 (1, 2). Consequently, the extended-release CAB nanosuspension, administered as an IM injection (600 mg in 3 mL) once every two months, was approved by the US FDA (Dec. 21, 2021) for HIV-1 PrEP. An injectable nanoparticle suspension of CAB (200 mg mL⁻¹) combined with the non-nucleoside reverse transcriptase inhibitor (NNRTI) rilpivirine (RPV, 300 mg mL⁻¹) became the first LA regimen for HIV treatment to receive approval by the US FDA (Jan. 21, 2021). The product is administered once-monthly as two separate intramuscular continuation injections (2 mL of each formulation) at separate gluteal sites. While these efforts are encouraging, they do not take advantage of the full portfolio of antiretroviral (ARV) agents currently available, especially drugs from the established, backbone NRTI mechanistic class.

It is believed by many in the field that, as in HIV-1 treatment, multiple agents are needed for effective, long-term HIV-1 PrEP (e.g., oral TDF-FTC and TAF-FTC), further complicating the development of viable long-acting candidates. An oral formulation of DTG, a second-generation ISTI with a high barrier to resistance, in combination with the established NRTI lamivudine (3TC) constitutes the first FDA-approved 2-drug cART regimen for the treatment of HIV. Both drugs have excellent, well-established safety profiles, including in adolescent and pediatric populations, and are recommended for use in pregnancy by the NIH's Office of AIDS Research.

There are no reports on 3TC or DTG subdermal implants. In situforming implants are an established drug delivery technology with a 30-year history yielding several FDA-approved products. The most common approach (“Atrigel”) relies on subcutaneous injection of a biodegradable polymer (usually PLGA or PLA) dissolved in an organic solvent (usually NMP or DMSO) and the active pharmaceutical ingredient (API). The Atrigel system has been used to deliver DTG in mice and macaques (3). There are important drawbacks associated with this platform, including: initial burst drug release, toxicity of the organic solvent(s), and local inflammation caused by the low molecular weight oligomers and acids that form as the polymer degrades.

DTG is potent in vitro against HIV-1, with an IC₅₀ in peripheral blood mononuclear cells (PBMCs) of 0.51 nM, a protein-adjusted IC₉₀ (PA-IC₉₀) of 0.15 μM (64 ng mL⁻¹),(4) and a long systemic half-life (12-15 h) (4, 5). For 3TC, in vitro EC₅₀ values spanned a wide range (0.003-15 μM)(6) and the elimination half-life in humans ranged from 5-7 h.(7) An implant maintaining steady-state DTG plasma concentrations above the PA-IC₅₀ for 6 months is theoretically feasible, but multiple devices may be required. The potency and PKs of 3TC make long-acting delivery via implant or injection unviable.

Enhancing DTG and 3TC potency for subdermal implant delivery is a highly promising HIV-1 PrEP strategy. Prodrugs can increase the potency of the parent agent, extend its in vivo half-life, and lead to tunability in important formulation parameters such as solubility. This design flexibility has been exploited for a number of small-molecule prodrugs in marketed, sustained-release injectable formulations, as described in (8) included herein in its entirety. While established ARV agents have been converted into prodrugs for injectable long-acting drug delivery, this mainly involved conjugation with fatty acids to dramatically decrease drug solubility, facilitating nanoparticle formulation. These approaches do not necessarily improve potency. Sillman et al. conjugated DTG with myristoyl chloride to afford the corresponding ester prodrug, MDTG (9). While MDTG was 8.7 times less water-soluble than DTG, an important attribute for nanoparticle formulation, the IC₅₀ of both agents against HIV-1 were not statistically different.

The enhanced potency prodrug strategies disclosed here use a library of peptides as drug conjugation moieties for cell targeting and increased cellular penetration. This approach is designed to, e.g., enable long-acting parenteral drug delivery and extends to other indications beyond HIV-1 prevention and treatment.

There remains a need for a more economical, practical, and efficient way of producing and manufacturing drug delivery systems that can be used locally or systemically, in solid or semi-solid formulations. The current disclosure is generally in the field of long-acting drug delivery devices, and more particularly in the field of devices for the controlled release of a drug from a device implantable or injectable in a body lumen or cavity, or subcutaneously, transcutaneously, or intravaginally.

### SUMMARY

Provided herein are drug delivery devices comprising one or more peptide-drug conjugates, wherein the device comprises (a) one or more kernels comprising the peptide-drug conjugate; and (b) one or more skins comprising a continuous membrane; wherein the one or more kernels and/or the one or more skins comprise defined pores which are not produced mechanically, and wherein the peptide-drug conjugate comprises a cell-penetrating peptide or cell-targeting peptide covalently linked to one or more drugs via one or more linkers. In some embodiments, the drug delivery devices comprise: (a) one or more drug-peptide conjugates, or prodrugs; (b) one or more kernels comprising one or more drug-peptide conjugates and optionally one or more additional pharmacologically active agents; and (c) one or more skins comprising a continuous membrane; wherein the one or more kernels and/or the skin comprises defined pores, and wherein the pores are not produced mechanically. In some embodiments, the reservoir kernel comprises a paste comprising one or more APIs. In some embodiments, the kernel comprises a fiber-based carrier. In some embodiments, the kernel comprises a porous sponge. In some embodiments, the drug-peptide conjugate is injected as a long-acting formulation. In other embodiments, drug-peptide conjugate is administered as a microarray patch, also known in the art as microneedle patches.

Also provided are drug delivery devices for implantation or injection into the body of a patient. In some embodiments, the device further comprises a shape adapted to be disposed within the body of a patient. In some embodiments, the device is capsule-shaped. In some embodiments, the device is in the shape of a torus. In some embodiments, the device comprises one or more cylindrical core elements disposed within a first skin, wherein the core elements comprise a kernel and optionally a second skin.

Further provided are methods of delivering one or more APIs to a patient in need thereof, comprising implanting a device disclosed herein into the patient's body. In some embodiments, the disclosure further provides methods of providing sustained, long term release of an API to a patient using the materials and methods described herein.

### BRIEF DESCRIPTION OF THE FIGURES

FIG. 1 shows a synthetic strategy to preparing peptide prodrugs using orthogonal coupling. R, resin backbone; L₁ and L₂, linkers; P, peptide; D, drug.

FIG. 2 shows a synthetic strategy to preparing peptide prodrugs using direct N-terminal coupling. R, resin backbone; L₁ and L₂, linkers; P, peptide; D, drug.

FIG. 3 shows a nonlimiting, general example of D-L₂-P construct involving a labile ester bond at D, and amide bond at P; 1≤n≤20.

FIG. 4 shows a nonlimiting example where linker L₂ is derived from succinic anhydride.

FIG. 5 shows PEGylated linker, L₂, conjugated to peptide P via an ester bond and drug D via a carbamate bond.

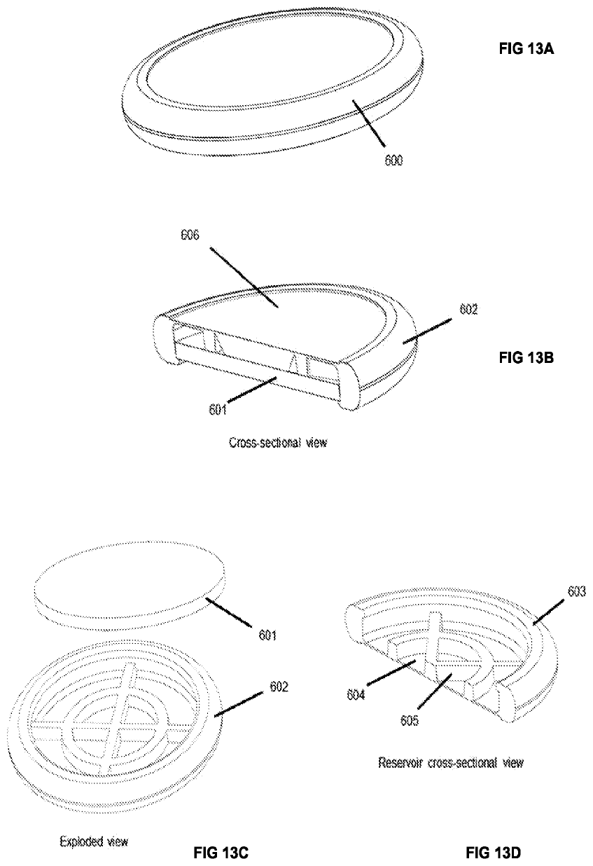

FIG. 6 shows a synthetic strategy to preparing peptide prodrugs using orthogonal coupling post cleavage from the resin. L₂, linkers P, peptide; D, drug. X=H, NH₂, Na, K, NH₄⁺, or other pharmaceutically acceptable salts; Y=H, Ac, Pyr, or other pharmaceutically acceptable salts.

FIG. 7 shows a synthetic strategy to preparing peptide prodrugs using direct N-terminal coupling post cleavage from the resin. L₂, linkers P, peptide; D, drug. X=H, NH₂, Na, K, NH₄⁺, or other pharmaceutically acceptable salts.

FIG. 8 shows a synthetic strategy to preparing peptide prodrugs using direct C-terminal coupling post cleavage from the resin. L₂, linkers P, peptide; D, drug. X=H, NH₂, Na, K, NH₄⁺; Y=H, Ac, Pyr. Y=H, Ac, Pyr, or other pharmaceutically acceptable salts.

FIG. 9 shows N-terminal thiol-maleimide conjugation.

FIGS. 10A and 10B shows a general peptide antiretroviral (ARV) prodrug synthesis strategy; Rink amide resin backbone; aspartic acid linker; peptide conjugate; R-group denotes ARV drug fragment. (i) Orthogonal O-allyl deprotection with cat. Pd(PPh₃)₄, phenylsilane, DCM; (ii) esterification with R—OH using N,N′-diisopropylcarbodiimide, 4-dimethylaminopyridine, DMF; (iii) N-terminus deprotection with piperidine in DMF followed by cleavage from the resin using TFA-triisopropylsilane-water. Inset box shows both ARV drug backbones, corresponding to the R-group. The primary amino group on 3TC is 4,4′-dimethoxytrityl- (DMTr) protected as N-DMTr-3TC; this group is removed with all the orthogonal amino acid protecting groups during cleavage. In FIG. 10A the aspartic acid linker is located at the C-terminus. In FIG. 10B the aspartic acid linker is located at the N-terminus. While the scheme is described in the context of antiretroviral drugs, the process is applicable to other types of drugs.

FIG. 11 shows a screening decision tree for ARV prodrug lead candidate selection. [prodrug]ₜₒₓ, onset (>10%) of cytotoxicity.

FIG. 12 shows exemplary embodiments of subdermal or intramuscular implant designs.

FIGS. 13A, 13B, 13C, and 13D show an exemplary embodiment of a single-membrane capsule-shaped implant design.

FIGS. 14A, 14B, 14C, 14D, 14E, 14F, and 14G show an alternative exemplary embodiment of a single-membrane capsule-shaped implant design.

FIGS. 15A, 15B, 15C, 15D, and 15E show an exemplary embodiment of a dual-membrane capsule-shaped implant design.

FIGS. 16A and 16B show an exemplary embodiment of an alternative disk design for a capsule-shaped implant design.

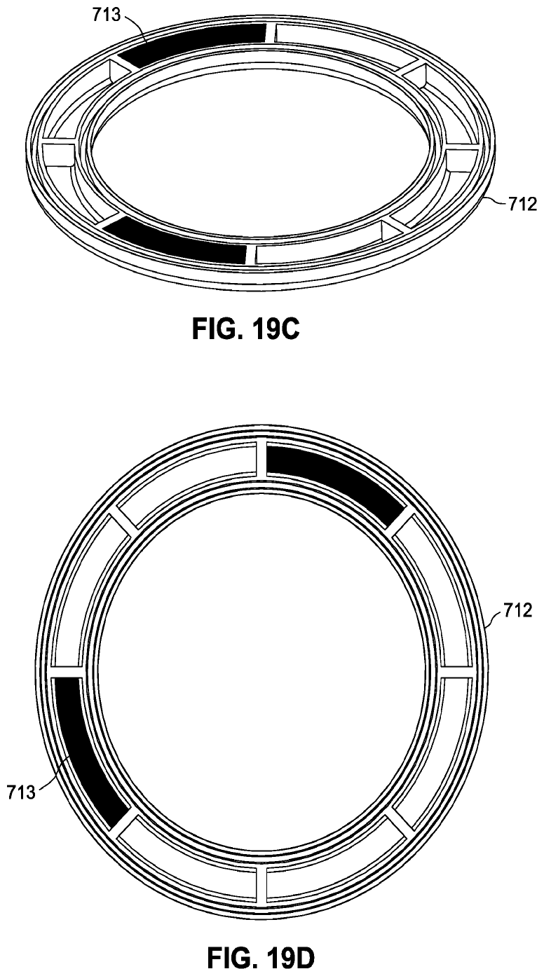

FIG. 17 shows exemplary embodiments of intravaginal ring designs.

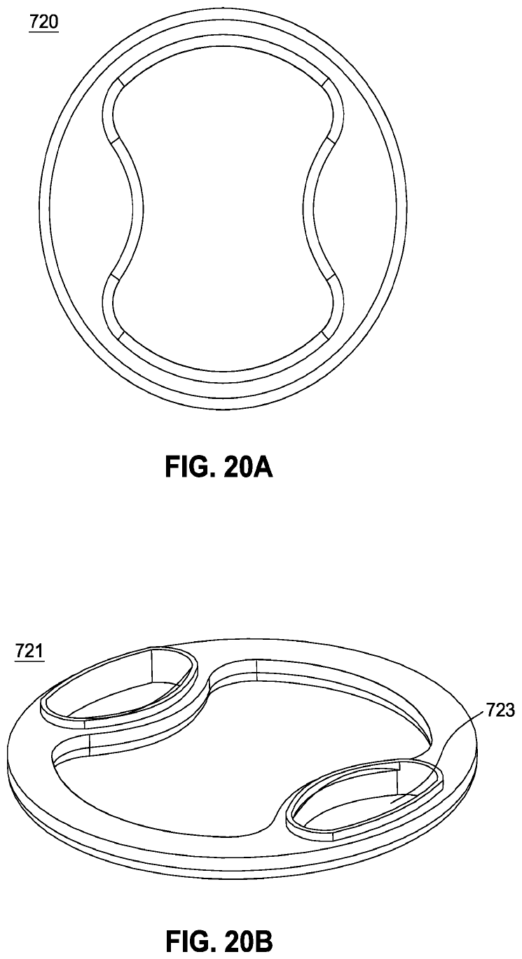

FIGS. 18A, 18B, 18C, and 18D show an alternative exemplary embodiment of an intravaginal ring design with a cylindrical kernel/skin inside a perforated carrier scaffold

FIGS. 19A, 19B, 19C, and 19D show an alternative exemplary embodiment of an intravaginal ring design with discrete compartments for peptide-drug conjugates, and/or APIs.

FIGS. 20A, 20B, 20C, 20D, and 20E show an alternative exemplary embodiment of an intravaginal ring design with discrete compartments for peptide-drug conjugates, and/or APIs in a non-toroidal geometry.

FIGS. 21A, 21B, 21C, 21D, and 21E show an alternative exemplary embodiment of a non-circular cross-section intravaginal ring design with discrete compartments for peptide-drug conjugates, and/or APIs and separate skins.

FIG. 22 shows exemplary embodiments of pessary ring designs.

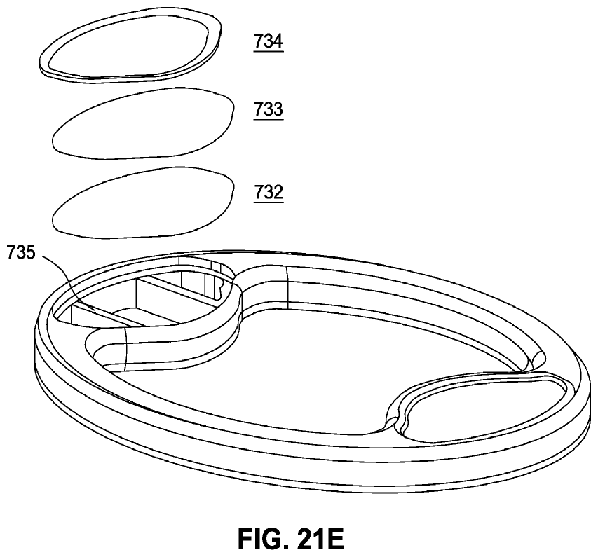

FIG. 23 shows exemplary embodiments of intrauterine device (IUD) designs.

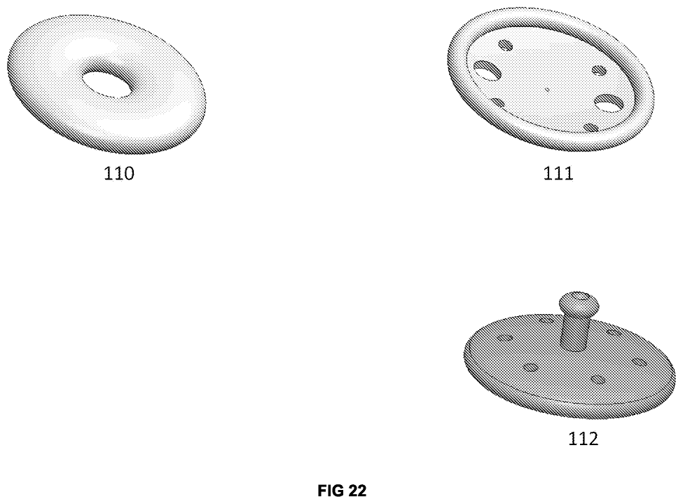

FIG. 24 shows exemplary embodiments of matrix implant designs.

FIG. 25 shows exemplary embodiments of matrix implant designs consisting of multiple kernels.

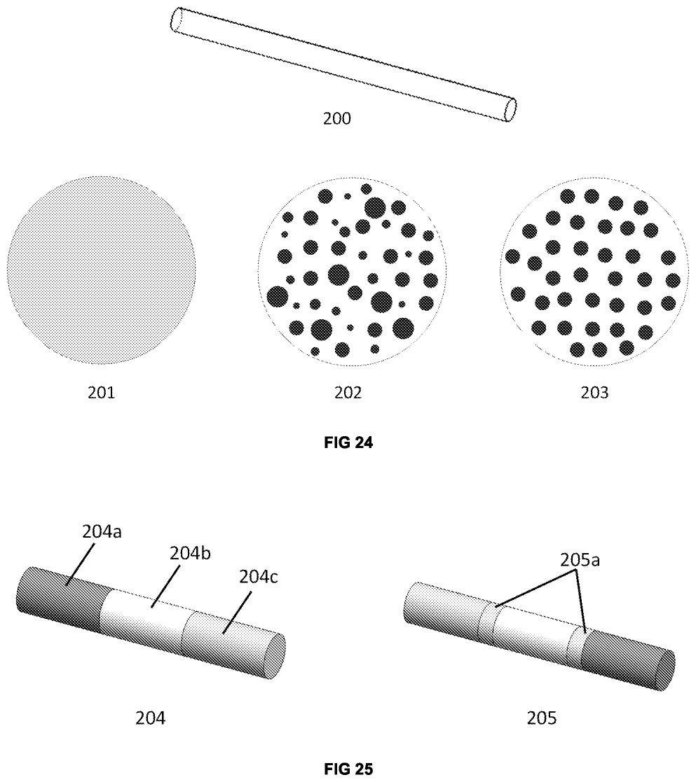

FIG. 26 shows exemplary embodiments of reservoir implant designs.

FIG. 27 shows exemplary embodiments of reservoir implant designs.

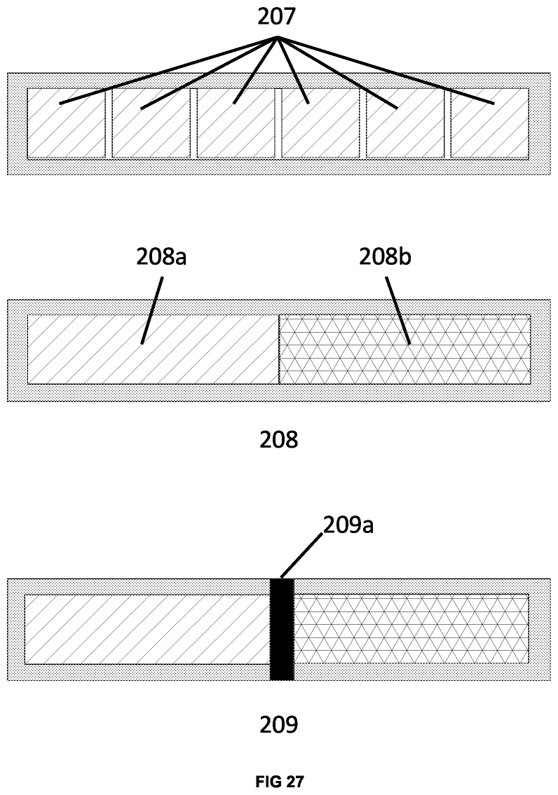

FIG. 28 shows exemplary embodiments of implant designs with a variety of external skins.

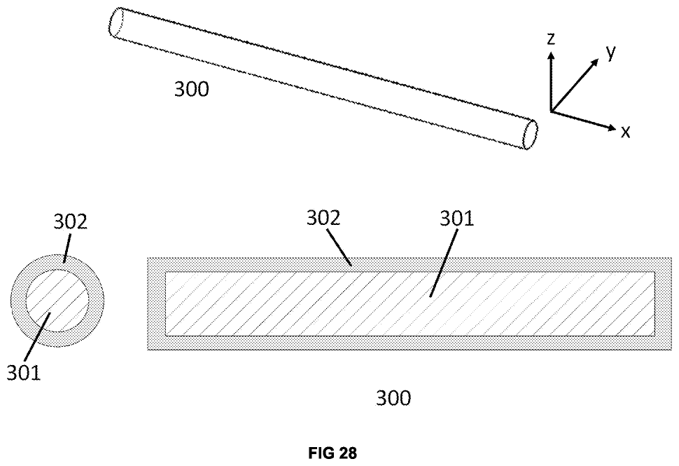

FIG. 29 shows exemplary embodiments of implant designs with a variety of external skins.

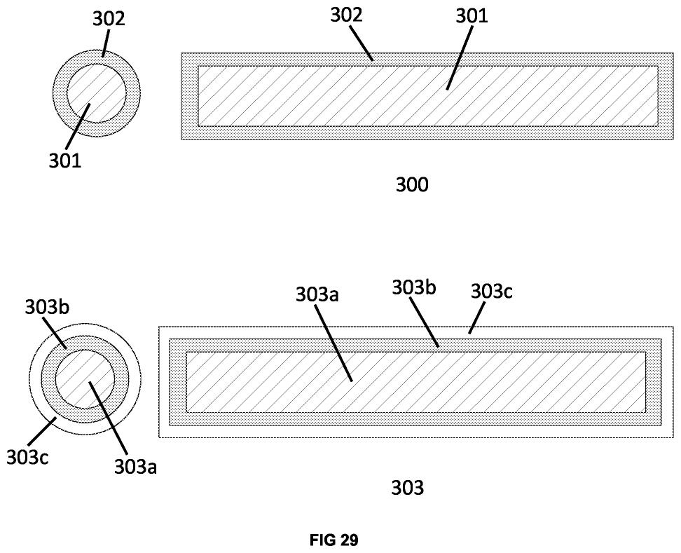

FIG. 30 shows exemplary embodiments of implant designs with a variety of external skins.

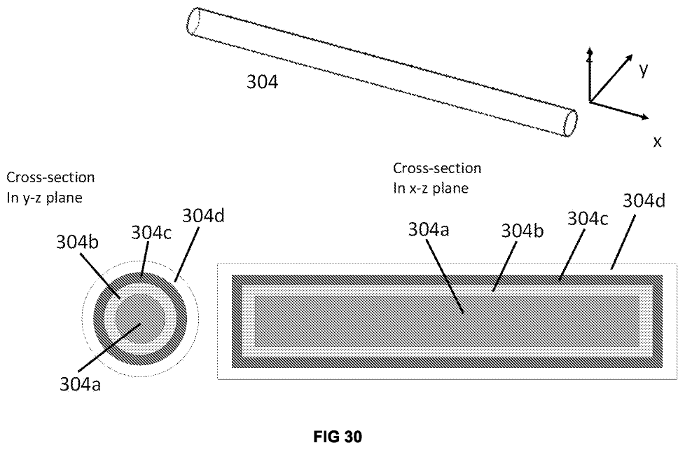

FIG. 31 shows exemplary embodiments of implant designs with a variety of kernels and external skins.

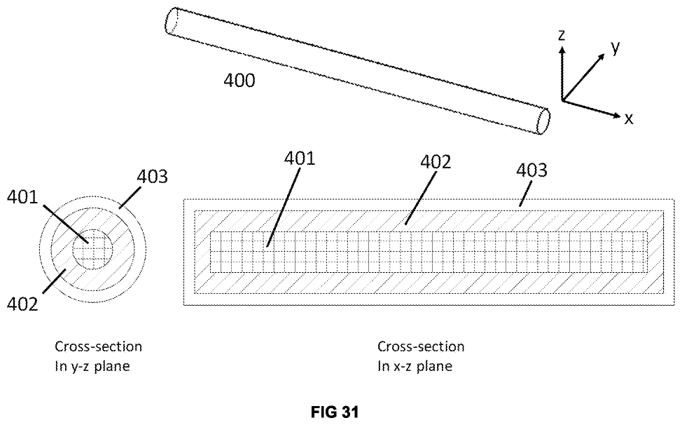

FIG. 32 shows exemplary embodiments of implant designs with a variety of kernels and external skins.

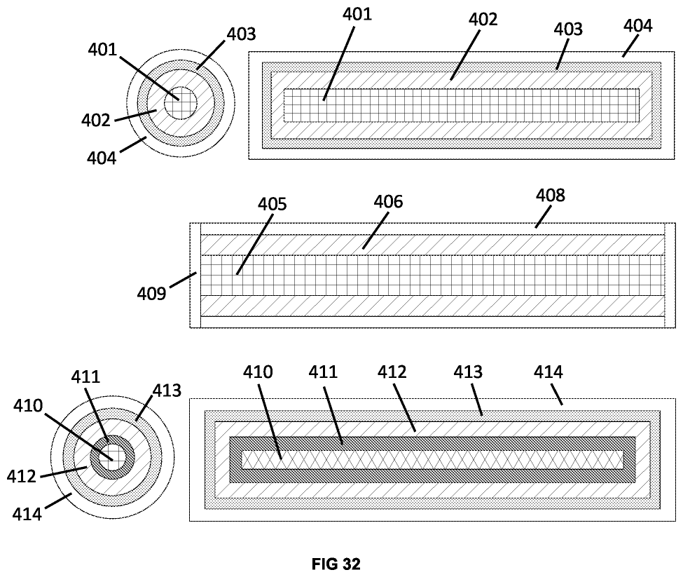

FIG. 33 shows exemplary embodiments of implant plugs.

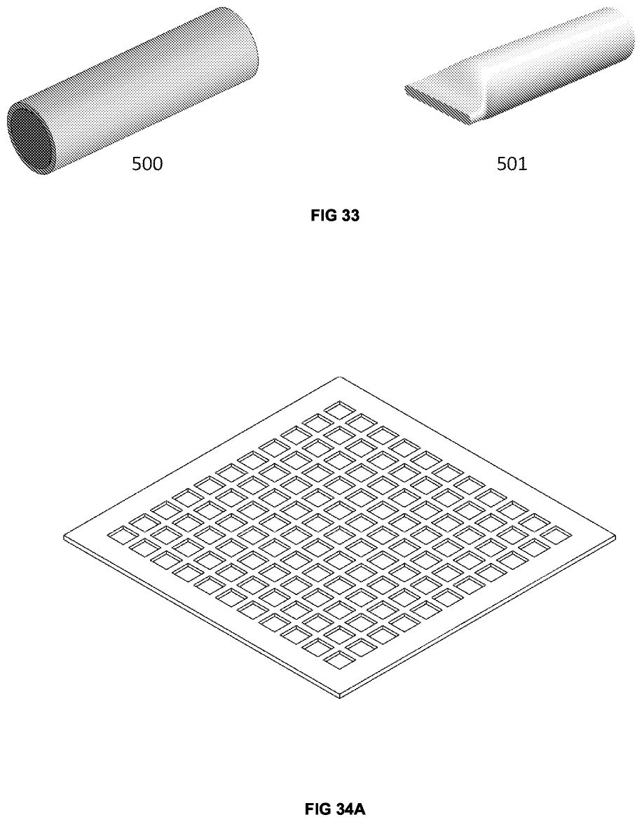

FIGS. 34A and 34B show drawings of patterned silicone skins formed by microlithography. Skins are shown with FIG. 35A square (1.5×1.5 mm) and FIG. 35B hexagonal (1.15 mm sides) grid support structures. The support grid walls are 500 μm wide and 250 μm high. The skin thickness exposed for drug diffusion (between the grid walls) is 100 μm.

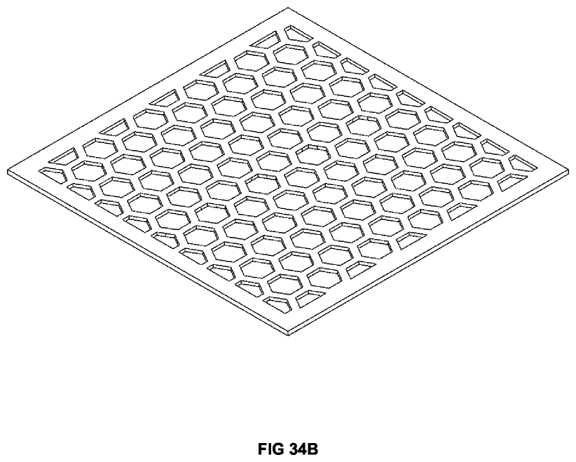

FIGS. 35A and 35B show XRD spectra of monoolein-water semisolid gels. FIG. 35A contains 20% w/w water, affording a main peak at 1.96°, corresponding to channels 4.50 nm in diameter. FIG. 35B contains 30% w/w water, affording a main peak at 1.8°, corresponding to channels 4.8 nm in diameter.

FIG. 36 shows preliminary implant prototype solid models and specifications. OD, outside diameter; L, outside length; H, outside height; W, outside width; V, capacity.

FIGS. 37A, 37B, and 37C show how ePTFE membranes lead to linear and controlled drug release. FIG. 37A AFM image of ePTFE membrane shows micro/nano-porous structure based on nodes (n) and fibrils (arrowhead). Scale bar, 5 μm. FIGS. 37B and 37C In vitro release studies into 1×PBS compare ePTFE membrane types, APIs, and excipients; FIG. 37B BSA release, from a depot of 33% w/v BSA in monoolein, is influenced by ePTFE membrane processing; unsintered, 0.24 mm ePTFE (0.75 mg d⁻¹); 0.17 mm, sintered ePFTE (0.24 mg d⁻¹). FIG. 37C: hIgG release is controlled by the excipients without modifying the ePTFE membrane; castor oil-monoolein, 4:1 (4.1 mg d⁻¹); monoolein-lecithin-medium chain triglycerides, 5:1.8:3.2 (0.87 mg d⁻¹).

### DETAILED DESCRIPTION

Provided herein are drug delivery devices comprising one or more peptide-drug conjugates, wherein the device comprises (a) one or more kernels comprising the peptide-drug conjugate; and (b) one or more skins comprising a continuous membrane; wherein the one or more kernels and/or skin comprises defined pores which are not produced mechanically, and wherein the peptide-drug conjugate comprises a cell-penetrating peptide or cell-targeting peptide covalently linked to one or more drugs via one or more linkers.

All references cited herein are incorporated by reference in their entirety as though fully set forth. Unless defined otherwise, technical and scientific terms used herein have the same meaning as commonly understood by one of ordinary skill in the art to which this disclosure belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (Boca Raton, FL, 2008); Oxford Textbook of Medicine, Oxford Univ. Press (Oxford, England, UK, May 2010, with 2018 update); Harrison's Principles of Internal Medicine, Vol. 1 and 2, 20^{th} ed., McGraw-Hill (New York, NY, 2018); Singleton and Sainsbury, Dictionary of Microbiology and Molecular Biology, 3^{rd} ed., revised ed., J. Wiley & Sons (New York, NY, 2006); Smith, March's Advanced Organic Chemistry Reactions, Mechanisms and Structure 7^{th} ed., J. Wiley & Sons (New York, NY, 2013); and Singleton, Dictionary of DNA and Genome Technology, 3^{rd} ed., Wiley-Blackwell (Hoboken, NJ, 2012), provide one skilled in the art with a general guide to many of the terms used in the present application.

One skilled in the art will recognize many methods and materials similar or equivalent to those described herein, which could be used in the practice of the present disclosure. Indeed, the present disclosure is in no way limited to the methods and materials described. For purposes of the present disclosure, certain terms are defined below.

“Treatment” and “prevention” and related terminology include, but are not limited to, treating, preventing, reducing the likelihood of having, reducing the severity of, and/or slowing the progression of a medical condition in a subject, termed “application” hereunder. Such conditions or applications can be remedied through the use of one or more agents administered through a sustained release agent delivery device.

These conditions, or applications, are described further under “Use and Applications of the Device” and may include, but are in no way limited to, infectious diseases (e.g., a human immunodeficiency virus (HIV) infection, acquired immune deficiency syndrome (AIDS), a herpes simplex virus (HSV) infection, a hepatitis virus infection, respiratory viral infections (including but not limited to influenza viruses and coronaviruses, for example SARS-CoV-2), tuberculosis, other bacterial infections, and malaria), diabetes, cardiovascular disorders, cancers, autoimmune diseases, central nervous system (CNS) conditions, and analogous conditions in non-human mammals.

In addition, the disclosure provides the administration of biologics, such as proteins and peptides, for the treatment or prevention of a variety of disorders such as conditions treatable with leuprolide (e.g., anemia caused by bleeding from uterine leiomyomas, fibroid tumors in the uterus, cancer of the prostate, and central precocious puberty), exenatide for the treatment of diabetes, histrelin acetate for the treatment for central precocious puberty, etc. A more detailed list of illustrative examples of potential applications of the disclosure is provided under “Use and Applications of the Device”.

As used herein, the term “HIV” includes HIV-1 and HIV-2.

As used herein, the term “agent” includes any, including, but not limited to, any drug or prodrug.

As used herein, the term “drug”, “medicament”, and “therapeutic agent” are used interchangeably.

As used herein, the term “API” means active pharmaceutical ingredient, which includes agents described herein. The term “API” includes drugs as well as peptide-drug conjugates and peptide prodrugs.

As used herein, the terms “peptide-drug conjugate” and “peptide prodrug” are used interchangeably and mean a drug that is covalently bound to a peptide with a goal of, e.g., enhancing the drug's pharmacologic properties.

The terms “drug delivery device” and “implant” and or “injectable” are used interchangeably herein, unless otherwise indicated, and include devices used, e.g., intravaginally, subcutaneously, intramuscularly, intraocularly, in the ear, brain, oral cavity, in the nasal cavity, or in any other body compartment.

As used herein, the term “IVR” means intravaginal ring, which includes embodiments described herein.

As used herein, the term “microarray patch” and “microneedle patch” are used interchangeably and are comprised of a plurality of micrometer-sized projections that can painlessly puncture the skin and access the epidermal/dermal layer to deliver the peptide-drug conjugate(s).

“Kernel” is defined as one or more compartments that contain one or more peptide-drug conjugates optionally in combination with one or more APIs and makes up the majority of the device volume.

“Matrix system” is a specific type of kernel defined as a system wherein one or more peptide-drug conjugates is uniformly distributed in the matrix material and has no other release barrier than diffusion out of the matrix material.

“Reservoir system” is a specific type of kernel defined as a system wherein one or more peptide-drug conjugates are formulated with excipients into a central compartment.

“Skin” is defined by a low volume element of the drug delivery system that covers part or all of a kernel. In some cases, the skin means the outer portion of the drug delivery system that contacts the external environment. The terms “skin”, “membrane”, and “layer” are used herein interchangeably.

“Rate limiting skin” is a specific embodiment of a skin defined by the part of the system which comprises of polymer(s) with relatively low permeability for the therapeutic agents.

The terms “patient” and “subject” are used interchangeably herein, and refer to a human or a non-human mammal.

“Permeability” means the measurement of a therapeutic agent's ability to pass through a thermoplastic polymer.

“Mammal,” as used herein, refers to any member of the class Mammalia, including, without limitation, humans and nonhuman primates such as chimpanzees and other apes and monkey species; farm animals such as cattle, sheep, pigs, goats and horses; domesticated mammals, such as dogs and cats; laboratory animals including rodents such as mice, rats and guinea pigs, and the like. The term does not denote a particular age or sex. Thus, adult and newborn mammals, whether male or female, are intended to be included within the scope of this term.

In various embodiments, the disclosure teaches devices, systems and methods for treating, preventing, reducing the likelihood of having, reducing the severity of and/or slowing the progression of a condition in a subject.

### Peptide-Drug Conjugates

The disclosure provides peptide prodrugs, also referred to as peptide-drug conjugates, to enhance the pharmacological properties of the parent agent. Exemplary embodiments are described below.

The pharmacological properties that the peptide-drug conjugates enhance relative to the parent drug may include, but are not limited to: increased potency for the target indication; increased in vivo half-life; increased accumulation in the target physiologic compartment; and reduced toxicity. The primary mode of delivery contemplated for the peptide-drug conjugates is parenteral. Targeting molecular transporter proteins for increased oral bioavailability—as well-known in the art (e.g., valacyclovir) for amino acid, dipeptide, or tripeptide drug conjugates targeting human peptide transporter 1 (PEPT1) in the small intestinal lumen and PEPT2 kidney proximal tubule—is not contemplated herein.

The peptide component of the peptide-drug conjugates (peptide prodrugs) can be synthesized using techniques and reagents known in the art. In one embodiment, automated, 9-fluorenylmethoxycarbonyl (Fmoc), Rink amide resin, solid-phase synthesis techniques are employed. In another embodiment, tert-butyloxycarbonyl (Boc) solid-phase synthesis techniques are employed. The methods (e.g., automated microwave-assisted peptide synthesis) and reagents (i.e., resins, coupling agents, cleavage chemistry) are well-known in the art.

### Peptides—General Features

The peptides that form the basis of the peptide-drug conjugates comprise linear or cyclic peptides. In some cases, the peptide is a linear peptide. In some cases, the peptide is a cyclic peptide. The peptides comprise amino acid sequences of 3-30 amino acids, preferably 4-20 amino acids. In one embodiment, the amino acids belong to the 20 natural amino acids known in the art, and are L-isomers. In another embodiment, the amino acids belong to the 20 natural amino acids known in the art, and are D-isomers. In another embodiment, one or more amino acids in the peptide sequences are unnatural amino acids known in the art, including, but not limited to protected N-methyl amino acids, L-indoline-2-carboxylic acid, L-2-cyclohexylglycine. Representative unnatural amino acid building blocks for peptide synthesis are standard catalog items from commercial vendors (e.g., Chem-Impex International, Inc., Wood Dale, IL, USA).

The peptides used to form drug conjugates of enhanced pharmacological properties are from two main classes: (1) cell-penetrating peptides; and (2) cell-targeting peptides, as described in more detail below. In some cases, the peptide comprises a cell-penetrating peptide. In some cases, the peptide comprises a cell-targeting peptide. In some cases, the peptide is an immunopotentiating peptide.

Non-limiting examples of suitable peptides include those listed in Table A below:

TABLE A
Peptide Sequence

AHLPIVRASLPS (SEQ ID NO: 1)
KGVSLSYRKKGVSLSYR (SEQ ID NO: 2)
KGVSLSYR (SEQ ID NO: 3)
YSAYPDSVPMMS (SEQ ID NO: 4)
SWLAYPGAVSYR (SEQ ID NO: 5)
CHVLWSTRC (SEQ ID NO: 6)
CVSNPRWKC (SEQ ID NO: 7)
QEYSPNLWGHEFRSH (SEQ ID NO: 8)
QEFSPNLWGLEFQKN (SEQ ID NO: 9)
QEFSPYMGLEFKKH (SEQ ID NO: 10)
KCCYSL (SEQ ID NO: 11)
AEGEFMYWGDSHWLQYWYEGDPAK (SEQ ID NO: 12)
MSRTMS (SEQ ID NO: 13)
MARAKE (SEQ ID NO: 14)
MARSGL (SEQ ID NO: 15)
NLYVASW (SEQ ID NO: 16)
TLTVLPW (SEQ ID NO: 17)
LTVSPWY (SEQ ID NO: 18)
BCDSDSDITWDQLWDLMK (SEQ ID NO: 19)
GSWYAWSPLVPSAQI (SEQ ID NO: 20)
RFRGLISLSQVYLSP (SEQ ID NO: 21)
HGRFILPWWYAFSPS (SEQ ID NO: 22)
PRNICSRRDPTCWTTY (SEQ ID NO: 23)
AYTKCSRQWRTCMTTH (SEQ ID NO: 24)
LTGTCLQYQSRCGNTR (SEQ ID NO: 25)
LYPLHTYTPLSLPLF (SEQ ID NO: 26)
PKMTLQRSNIRPSMP (SEQ ID NO: 27)
GVTDSSTSNLDMPHW (SEQ ID NO: 28)
APRPGPWLWSNADSV (SEQ ID NO: 29)
DKPTAFVSVYLKTAL (SEQ ID NO: 30)
SMEPALPDWWWKMFK (SEQ ID NO: 31)
PQNSKIPGPTFLDPH (SEQ ID NO: 32)
AEGEFGWWGDSHWLQYEGDPAK (SEQ ID NO: 33)
AEGEFGHWCDQHWLQYWYEGDPAK (SEQ ID NO: 34)
AEGEFLMWGGSHWLEYWYEGDPAK (SEQ ID NO: 35)
AEGEFPRWGDSHWLQYWYEGDPAK (SEQ ID NO: 36)
AEGEFIHNRYNRFFYWYGDPAK (SEQ ID NO: 37)
AEGEFWGDSHWLQYWYEGDPAK (SEQ ID NO: 38)
SVSVGMKPSPRP (SEQ ID NO: 39)
GNGRAHA (SEQ ID NO: 40)
CRGDKGPDC (SEQ ID NO: 41)
RGDGWK (SEQ ID NO: 42)
CDCRGDCFC (SEQ ID NO: 43)
CRGDCF (SEQ ID NO: 44)
RGDF (SEQ ID NO: 45)
GPLPLR (SEQ ID NO: 46)
CRGDK (SEQ ID NO: 47)
KDEPQRRSARLSAKPAPPKPEPKPKKAPAKKC (SEQ ID NO: 48)
KDEPQRRSARLSAKPAPPKPEPKPKKAPAKK (SEQ ID NO: 49)
KDEPQRRSARLSAKPAPPKPEPKPKKAPAKK (SEQ ID NO: 50)
CLDGGRPKC (SEQ ID NO: 51)
CLSDGKRKC (SEQ ID NO: 52)
LTLRWVGLMS (SEQ ID NO: 53)
KGVSLSYR (SEQ ID NO: 54)
TFFYGGSRGKRNNFKTEEY (SEQ ID NO: 55)
LRKLRKRLLR (SEQ ID NO: 56)
CNGRC (SEQ ID NO: 57)
(KKEEE)3K (SEQ ID NO: 58)
WAVGHL-NH2 (SEQ ID NO: 59)
Cyclic (RGD) (SEQ ID NO: 60)
VQIVYK (SEQ ID NO: 61)
KYLAYPDSVHIW (SEQ ID NO: 62)
SSKYQSL (SEQ ID NO: 63)
Cyclic RGDfK (SEQ ID NO: 64)
RGD (SEQ ID NO: 65)
QEXSP (SEQ ID NO: 66)
MXRXXX (SEQ ID NO: 67)
CSRXW (SEQ ID NO: 68)
AEGEF (SEQ ID NO: 69)
XKRX (SEQ ID NO: 70)
GRKKRRQRRRPPQ (SEQ ID NO: 71)
RKKRRQRRR (SEQ ID NO: 72)
TRQARRNRRRRWRERQR (SEQ ID NO: 73)
RRRRNRTRRNRRRVR (SEQ ID NO: 74)
RQIKIWFQNRRMKWKK (SEQ ID NO: 75)
GDCLPHLKLCKENKDCCSKKCKRRGTNIEKRCR (SEQ ID NO: 76)
GWTLNSAGYLLGKINLKALAALAKKIL (SEQ ID NO: 77)
PKKKRKV (SEQ ID NO: 78)
VQRKRQKLMP (SEQ ID NO: 79
SKKKKTKV (SEQ ID NO: 80)
GRKRKKRT (SEQ ID NO: 81)
ERKKRRRE (SEQ ID NO: 82)
FKKFRKF (SEQ ID NO: 83)
RRMKWKKK (SEQ ID NO: 84)
(R)n 4 ≤ n ≤ 12 (SEQ ID NO: 85)
(K)n 4 ≤ n ≤ 12 (SEQ ID NO: 86)
(P), 4 ≤ n ≤ 12 (SEQ ID NO: 87)
GIGAVLKVLTTGLPALISWIKRKRQQ (SEQ ID NO: 88)
GFCWYVCVYRNGVRVCYRRCN (SEQ ID NO: 89)
KETWWETWWTEWSQPKKKRKV (SEQ ID NO: 90)
KKTWWKTWWTKWSQPKKKRKV (SEQ ID NO: 91)
GALFLGFLGAAGSTMGAWSQPKKKRKV (SEQ ID NO: 92)
GLFRALLRLLRSLWRLLLRA (SEQ ID NO: 93)
HGLASTLTRWAHYNALIRAF (SEQ ID NO: 94)
PLIYLRLLRGQWC (SEQ ID NO: 95)
AGYLLGKINLKALAALAKKIL (SEQ ID NO: 96)
LIRLWSHLIHIWFQNRRLKWKKK (SEQ ID NO: 97)
MVTVLFRRLRIRRACGPPRVRV (SEQ ID NO: 98)
LGTYTQDFNKTFPQTAIGVGAP (SEQ ID NO: 99)
RGGRLSYSRRRFSTSTGR (SEQ ID NO: 100)
LLIILRRRIRKQAHAHSK (SEQ ID NO: 101)
KLALKLALKALKAALK (SEQ ID NO: 102)
KLAKLAKLA (SEQ ID NO: 103)
VRLPPPVRLPPPVRLPPP (SEQ ID NO: 104)
GLWRALWRLLRSLWRLLWRA (SEQ ID NO: 105)
CSIPPEVKFNKPFVYLI (SEQ ID NO: 106)
PFVYLI (SEQ ID NO: 107)
SDLWEMMMVSLACQY (SEQ ID NO: 108)
YTFGLKTSFNVQ (SEQ ID NO: 109)
AAVALLPAVLLALLP (SEQ ID NO: 110)
Cyclic-(GGVCPKILKKCRRDSDCPGACICRGNGYCGSGSD) (SEQ ID NO: 111)
Cyclic-GGVCPKILAACRRDSDCPGACICRGNGYCGSGSD (SEQ ID NO: 112)
Cyclic-(GGVCPAILKKCRRDSDCPGACICRGNGYCGSGSD) (SEQ ID NO: 113)
Cyclic-(GGVCPKILAKCRRDSDCPGACICRGNGYCGSGSD) (SEQ ID NO: 114)
Cyclic-(GGVCPKILKACRRDSDCPGACICRGNGYCGSGSD) (SEQ ID NO: 115)
Cyclic-(GLPVCGETCVGGTCNTPGCTCSWPVCTRN) (SEQ ID NO: 116)
Cyclic-(GLPVCGETCVGGTCNTPGCKCSWPVCTRN) (SEQ ID NO: 117)
Cyclic-(GLPVCGETCVGGTCNTPGCTCSWPKCTRN) (SEQ ID NO: 118)
Cyclic-(GRCTKSIPPICFPD) (SEQ ID NO: 119)

Peptide-drug conjugates of the disclosure leverage the native functional groups in the drug, peptide, and/or an optional linker amenable to derivatization. Conjugation strategies have been further described in the art (e.g., in references 10-13, incorporated herein in their entirety). Illustrative non-limiting functional groups suitable for derivatization include: esters, carbonates, phosphates and phosphonates (e.g., phosphonooxymethylethers, phosphoramidates, CycloSal, HepDirect, ProTide), acylsulfonamides, amides, carbamates, N-Mannich adducts, N-acyloxyalkylamines, azo conjugates, imines, hydrazones, sulfenamides, amidines, and guanidines.

In some embodiments, the drug of the peptide-drug conjugates of the disclosure comprises two or more conjugatable functional groups, which may be the same or different. In some cases, the conjugates include two or more peptides conjugated to the drug; the peptides may be the same or different. As a non-limiting example, the hydroxy group in 3TC can be conjugated to a first peptide, while the amino group can be conjugated to a second peptide via a carbamate, or vice versa.

### Synthesis

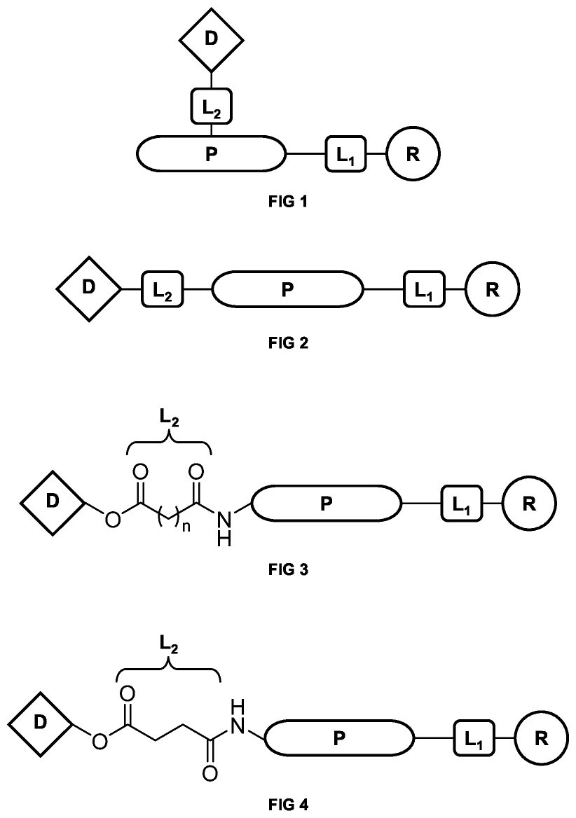

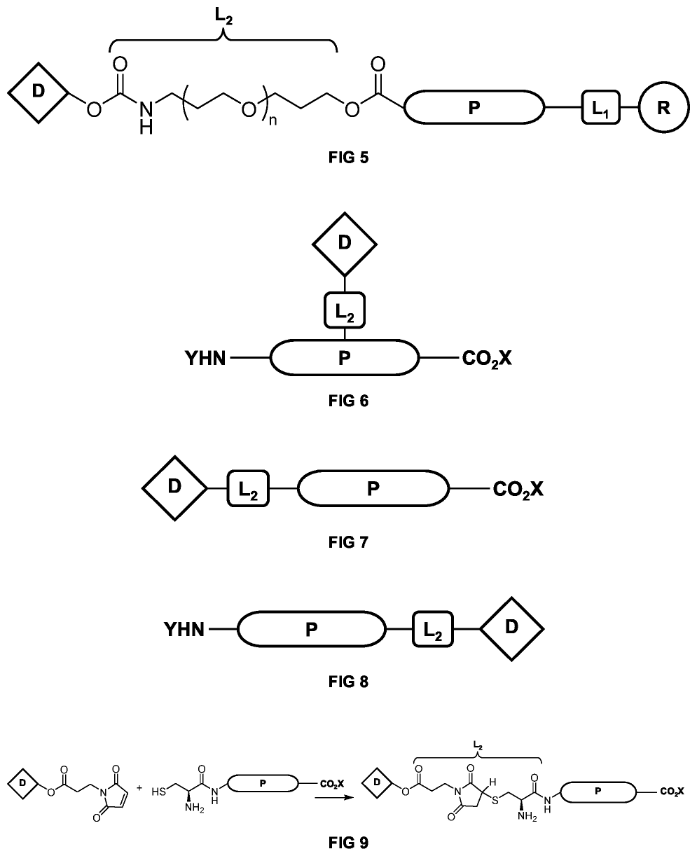

One general non-limiting synthetic strategy to preparing the peptide prodrugs described herein involves direct coupling of the peptide and the drug, as shown schematically in FIGS. 1-2.

In the strategies shown in FIGS. 1 and 2, linker L₁ anchors peptide P to the solid-phase resin, R, and L₂ links drug D directly to P via an in vivo chemically labile bond (e.g., ester, carbamate, carbonate). In one embodiment, L₁ forms part of the resin. There are numerous examples in the art of such L₁-R systems, as illustrated by standard catalog items from commercial vendors (e.g., Chem-Impex International, Inc., Wood Dale, IL, USA). For example, Rink amide resins will result in an amidated C-terminus, and the amide amino group is transferred to the peptide from the resin. In another example, a sulfonamide resin, such as 4-sulfamylbutyryl AM resin, is employed and once synthesis is completed, the peptide-resin conjugate is activated using iodoacetonitrile (ICH₂CN), followed by cleavage with ethyl mercaptoacetate (HSCH₂CH₂CO₂Et, H-SR) and acidolytic deprotection to give the fully deprotected peptide α-thioester. The thioester functionality (SR) at the C-terminus is transferred to the peptide during workup.

The term “linker”, as used herein, refers to a moiety comprising two reactive groups connected by one or more carbons or heteroatoms. In some cases, one or more linkers are covalently linked to a terminal residue of the peptide. In some cases, the terminal residue is an N-terminal residue. In some cases, the terminal residue is a C-terminal residue. In some cases, one or more linkers are covalently linked to a non-terminal residue of the peptide. In some cases, one or more linkers are covalently linked to a side chain of the non-terminal residue. In some cases, the non-terminal residue is a lysine, a cysteine, an aspartic acid, or a glutamic acid residue.

In some cases, one or more linkers are (C₁-C₈)alkyl, (C₁-C₈)alkenyl, (C₁-C₈)alkynyl, (C₃-C₁₀)cycloalkyl, (C₆-C₁₀)aryl, (C₂-C₈)heteroalkyl, or (C₂-C₈)heteroaryl; wherein alkyl, alkenyl, alkynyl, cycloalkyl, aryl or heteroaryl is optionally substituted by (C₁-C₆)alkyl, 1-4 halogens, (C₁-C₄)alkoxy, (C₁-C₄)alkoxycarbonyl, hydroxy, amino, (C₁-C₄)alkylamino, (C₁-C₄)dialkylamino, (C₃-C₁₀)cycloalkyl, (C₁-C₆)alkylcarbonyloxy, (C₁-C₆)alkylcarbonylamido, (C₁-C₄)alkylamidocarbonyl, (C₁-C₄)dialkylamidocarbonyl, nitro, cyano, (C₁-C₄)alkylimino, mercapto, or (C₁-C₄)alkylmercapto.

In some cases, one or more linkers have the structure:

—C(O)-J-C(O)—K—C(O)—,
wherein K is selected from —C₁-C₁₀alkylene-, —(CH₂),A-(CH₂)ₜ— and Q; —C(O)-J- is absent, an amino acid residue or a peptide of 2 to 10 amino acid residues,
wherein the —C(O)— is derived from the carboxy terminal of the amino acid or peptide;
A is —O—, —S—, —NR₁—, —N⁺(R₁)₂—, —S—S—, —[OCH₂CH₂]ᵣ—O—, -L-, or —O—Y—O—;
Q is L or —Z═N—NH—S(O)ᵥ-L-;
L is cycloalkyl, heterocycloalkyl, aryl, or heteroaryl;
Z is —(CH₂)_{w}—C(CH₃)═, —(CH₂)_{w}CH═, cycloalkyl, or heterocycloalkyl;
R₁ is hydrogen or C₁-C₄alkyl;
s and t are independently an integer selected from 1 and 2;
r is an integer selected from 1, 2, and 3;
v is an integer selected from 0, 1, and 2; and
w is an integer selected from 1, 2, 3, and 4.

In some cases, the one or more linkers comprise two or more amino acids, a peptide, a polyether such as polyethylene glycol (i.e., PEG), and/or combinations thereof. In some cases, the one or more linkers comprise two or more amino acids. In some cases, the one or more linkers are a peptide. In some cases, the one or more linkers comprise a polyether. In some cases, the one or more linkers comprise polyethylene glycol (i.e., PEG), and/or combinations thereof. A common linker is Gly-Ser-Gly (“GSG”) or multiples thereof (GSG)ₙ. In some cases, the one or more linkers comprise Gly-Ser-Gly (“GSG”). In applications where it is important to have longer distance between drug D and peptide P, polyethylene glycol (“PEG”) chains of varying lengths [i.e., (PEG)ₙ) are inserted between these two moieties.

In the embodiments shown in FIGS. 1-2, the drug, D, is coupled directly to the peptide during solid-phase synthesis; i.e., peptide is resin-anchored. Coupling is achieved using a suitable reactive chemical functionality on one of the orthogonal amino acid side-chains (FIG. 1) with a suitable reactive chemical functionality in the drug. In one non-limiting embodiment, the orthogonal carboxylic acid in aspartic acid (β-carboxyl group) and glutamic acid (γ-carboxyl group) can be coupled with an alcohol functionality in the drug to afford an ester. In one non-limiting embodiment, the orthogonal amino group in lysine (ε-amino group) can be coupled with an alcohol functionality in the drug to afford a carbamate, or a carboxylate functionality in the drug to afford an amide. In another, non-limiting embodiment, the orthogonal hydroxyl group in serine (p-hydroxyl group) can be coupled with an alcohol functionality in the drug to afford a carbonate, a carboxylate functionality in the drug to afford an ester, or an amino functionality in the drug to afford a carbamate. Coupling is achieved using synthetic methods and reagents well-known in the art, such as carbodiimides in the presence of activators (e.g., N-hydroxybenzotriazole) and/or catalysts (e.g., 4-dimethylaminopyridine) or 1,1′-carbonyldiimidazole. In an embodiment when drug D is coupled directly to an orthogonal reactive group in the peptide, selective deprotection of said reactive group is achieved using methods and reagents well-known in the art to avoid deprotection of the other reactive groups on the peptide side chains. In a non-limiting embodiment, the aspartic acid β-carboxyl group is protected as the O-allyl ester and is deprotected with catalytic tetrakis(triphenylphosphine)palladium(0) [Pd(PPh₃)₄] and phenylsilane in dichloromethane. In some cases, the one or more linkers are conjugated to the peptide and/or the drug via an ester, a carbonate, a carbamate moiety, a succinimidyl thioether, or a triazole moiety. In some cases, the one or more linkers are conjugated to the peptide and/or the drug via an ester, a carbonate, or a carbamate moiety. In some cases, the one or more linkers are conjugated to the peptide and/or the drug via an ester moiety. In some cases, the one or more linkers are conjugated to the peptide and/or the drug via a carbonate moiety. In some cases, the one or more linkers are conjugated to the peptide and/or the drug via a carbamate moiety. In some cases, the one or more linkers are conjugated to the peptide and/or the drug via a succinimidyl thioether or a triazole moiety. In some cases, the one or more linkers are conjugated to the peptide and/or the drug via a succinimidyl thioether moiety. In some cases, the one or more linkers are conjugated to the peptide and/or the drug via a triazole moiety.

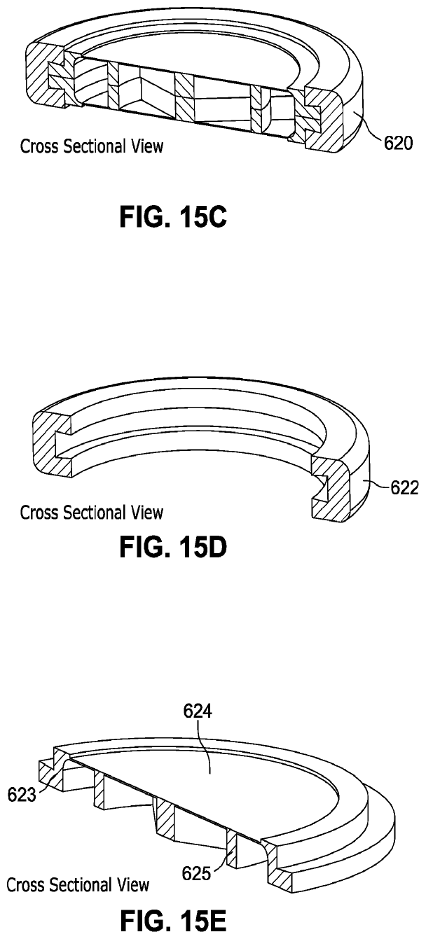

A nonlimiting example of a general peptide-ARV prodrug synthetic approach is shown in FIGS. 10A and 10B. An aspartic acid linker (shown in FIG. 10A on the C-terminus, and in FIG. 10B on the N-terminus) is added to all sequences shown in Table A for orthogonal conjugation to afford the corresponding ester prodrug. This innovative synthetic design allows peptide assembly followed by drug conjugation to be performed using automated, solid-phase synthesis and holds advantages in terms of simplicity and efficiency compared to performing the prodrug reaction step separately using traditional flask chemistry (i.e., additional protection/deprotection or intermediate purification steps). In this nonlimiting example, the peptide ARV drug conjugates are synthesized using automated, standard 9-fluorenylmethoxycarbonyl (Fmoc), Rink amide resin, solid-phase synthesis techniques.

In another nonlimiting embodiment, direct drug-peptide conjugation occurs at the resin-supported peptide N-terminus (FIG. 2).

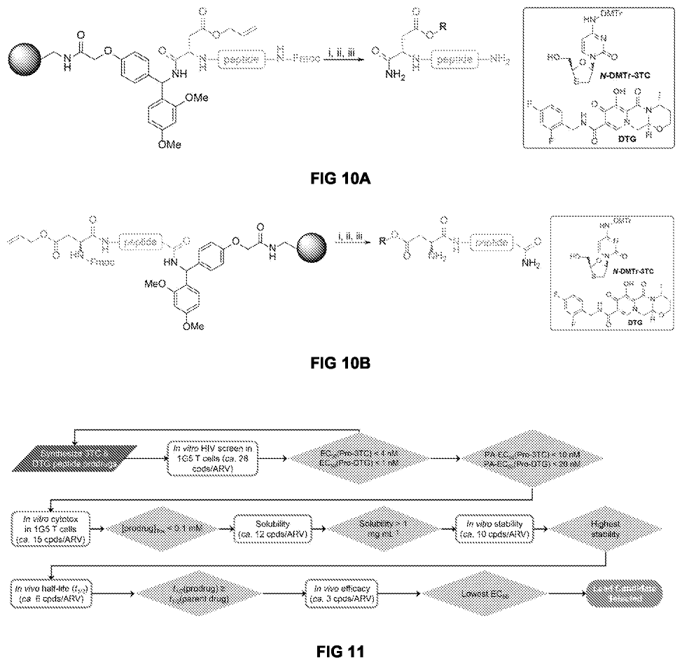

In another non-limiting embodiment involving on-resin coupling of drug, D, to peptide, P, through a linker e.g., L₂ (FIGS. 1-2), the linker consists of a molecular linker, bound to both D and the linker e.g., L via covalent bonds. In one embodiment, the bond between the linker e.g., L₂ and D is readily cleaved by enzymatic and/or chemical transformation in vivo to release the active parent drug, D (FIG. 3).

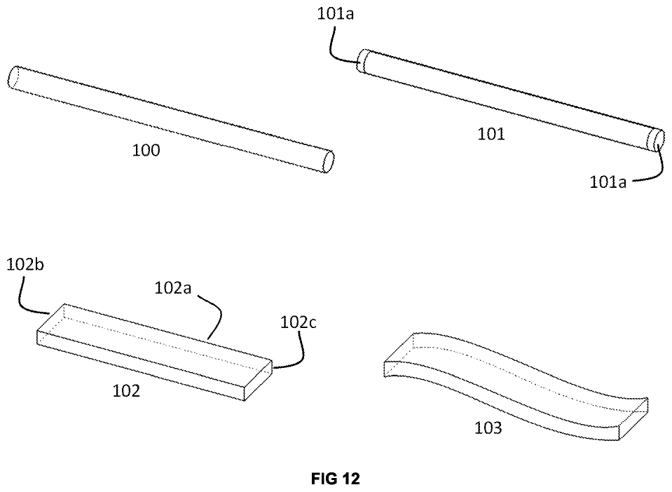

Nonlimiting examples of such bonds are ester, carbonate, and carbamate bonds. In some embodiments, the bond between the linker, e.g., L₂ and P can be readily cleaved by enzymatic and/or chemical transformation in vivo, or can be stable to in vivo cleavage such as some amides and triazoles. For example, a drug, D, containing a hydroxyl group can be reacted with succinic anhydride to afford the monoester of 1,4-butanedioic acid (succinic acid). The free carboxylic acid then can be condensed with an amino group (side chain or N-terminus) in peptide, P, to afford the corresponding amide (FIG. 4).

In another nonlimiting embodiment, the linker, e.g., L₂ contains a labile linkage to peptide, P, and a less labile linkage to drug, D. Under this embodiment, the peptide is hydrolyzed first, freeing the drug still chemically bound to the linker, which is later cleaved from the drug. In another embodiment, peptide P enhances delivery of drug D to one or more pharmacologic compartments before cleavage of P from the linker, e.g., L₂. Subsequently, the drug-linker, e.g., D-L₂ conjugate has superior pharmacologic properties compared to D alone. For example, a drug, D, contains a hydroxyl group that is conjugated to PEGylated linker the linker, e.g., L₂ via a carbamate group. A free carboxylic acid group in peptide P side chain or N-terminus (e.g., aspartate or glutamate) also is conjugated with linker the linker, e.g., L₂ via an ester bond. The ester bond is cleaved first in vivo, freeing the PEGylated drug carbamate.

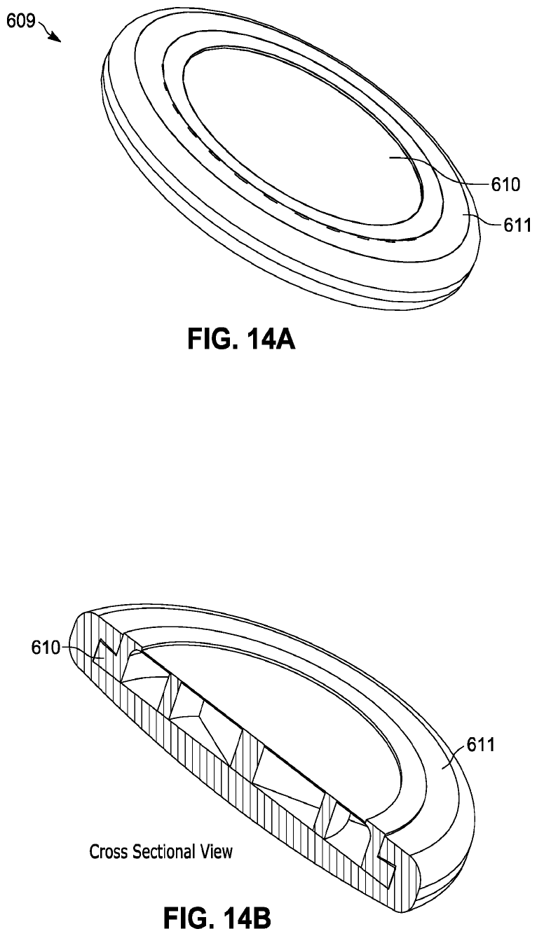

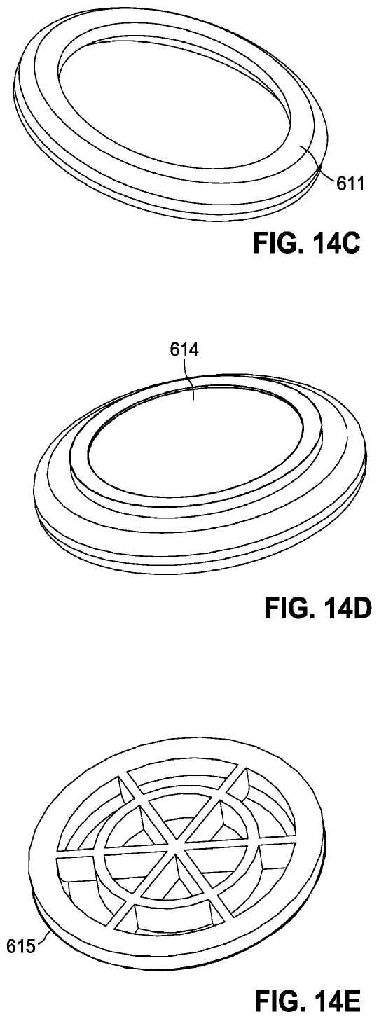

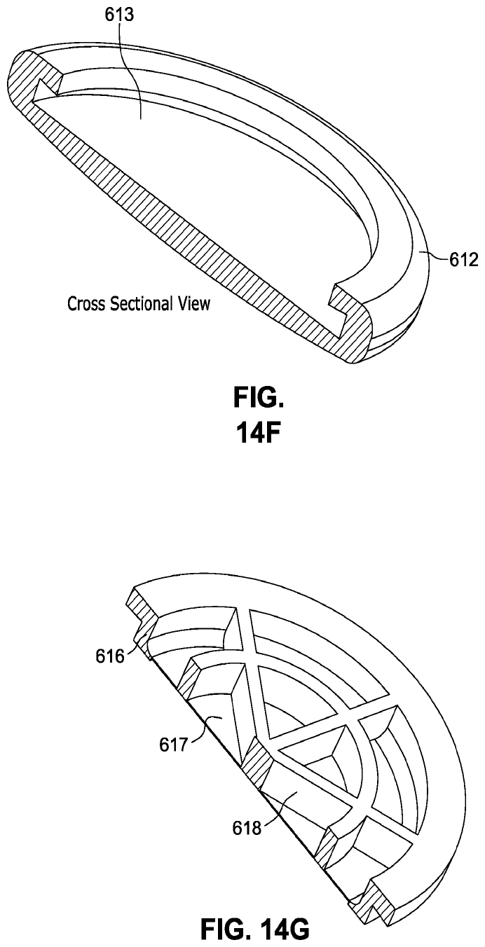

In another embodiment, a synthetic strategy to preparing the peptide prodrugs described herein involves drug-peptide conjugation after peptide cleavage from the resin, as shown in FIGS. 6-8.

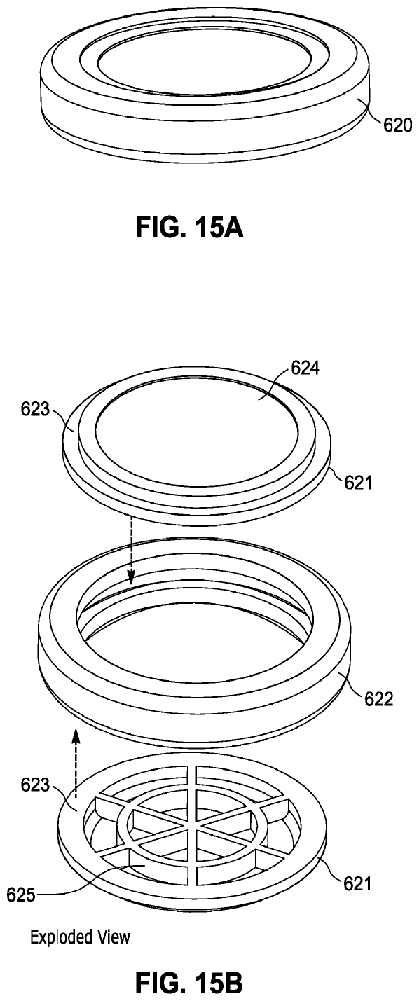

In these embodiments, the peptide-linker-drug coupling is achieved using chemoselective synthetic strategies known in the art to avoid side-reactions with the unprotected orthogonal reactive groups in peptide P. In one nonlimiting embodiment, coupling is achieved using so-called “click chemistry”. Click chemistry as applicable to the disclosed peptide-drug conjugates includes a functionality on peptide P that reacts with another, complementary functionality on drug D to afford a product linked through the linker, e.g., L₂. Nonlimiting examples of such click chemistry tools include:
copper-catalyzed reaction of an azide with an alkyne to afford the corresponding triazole; sterically strained trans-cycloalkene with azides, tetrazines and tetrazoles; or the reaction of a thiol (e.g., cysteine) with maleimide to afford the corresponding succinimidyl thioether. A non-limiting example is shown in FIG. 9.

### Peptide Classes and Sequences

In various aspects, the drug is conjugated or linked to a moiety that enhances delivery and/or penetration of the drug into target cells. In this regard, the disclosure provides use of a cell penetrating peptide to, e.g., enhance uptake of the drug in target cells. Cell penetrating peptides (CPPs), also known as protein transduction domains, are a class of amino acid sequences generally 3-30 residues in length which are capable of traversing biological membranes and delivering a wide variety of payloads to cells. A common (but not required) attribute of CPPs is a series of cationic amino acids in combination with a sequence which forms an α-helix at low pH. See, e.g., (14). Many CPPs have an amino acid composition that is rich in basic amino acids (e.g., lysine or arginine). Merely to illustrate, CPPs may include any combination of at least two arginines and at least two hydrophobic amino acids, such as a combination of two to three arginines and at least two hydrophobic amino acids, although this is not required.

In various aspects, the peptide of the peptide-drug conjugate is a cationic CPP, an amphipathic CPP, hydrophobic CPP, or a cyclic CPP. Exemplary peptides suitable for use as CPPs include, but are not limited to, protamine, nucleoline, spermine or spermidine, polylysine-containing peptides (peptides comprising, e.g., 3, 4, 5, 6, 7, 8, 9, 10, or more lysines), polyarginine-containing peptides (peptides comprising, e.g., 3, 4, 5, 6, 7, 8, 9, 10, or more arginines), polyproline-containing peptides (peptides comprising, e.g., 3, 4, 5, 6, 7, 8, 9, 10, or more prolines), Transportan, or MPG peptides, HIV-binding peptides, trans-activator of transcription (TAT)-derived peptides (including cysteine-free versions of TAT-derived peptides; residues 47-57 of HIV-1 TAT), members of the penetratin family (e.g., penetratin), Antennapedia-derived peptides (e.g., peptides derived from Drosophila antennapedia), antimicrobial-derived CPPs, SynB, PVEC, hCT-derived peptides, SAP, PPTG, VP22-derived peptides, and the like. In various aspects, the CPP is a cationic CPP, an amphipathic CPP, a hydrophobic CPP, and/or a cyclic CPP. Examples of CPPs are provided in TABLE A.

Optionally, the peptide of the peptide-drug conjugation is a cell targeting peptide (CTP). A “cell targeting peptide” refers to a peptide that recognizes and preferentially binds specific cells and tissues. In one embodiment, the cell targeting peptides target surface features of immune cells, and are referred to as immunopotentiating peptides. Similar to CPPs, CTPs are generally short peptides (e.g., 3-30 residues in length) and often derived from motifs which allow the peptide to target cell surface biological markers. In some examples, a CPP can also function as a CTP. In various aspects, the CTP targets, e.g., a cancer cell, an endothelial cell, a lipocyte, a cell of the blood-brain barrier, an immune cell, and the like. Examples of CTPs are provided herein in TABLE A. It will be appreciated that common motifs are shared by various CTPs which target a cell or tissue type. For example, CTPs which bind breast cancer cells may comprise the motif AEGEF (SEQ ID NO: 69), QEXSP (SEQ ID NO: 66) wherein X is Y or F, or MX1 RX2X3X4 wherein X1 is a polar uncharged amino acid, such as S, T, N, or Q, or a hydrophobic, non-aliphatic amino acid, such as A, V, I, L, or M; X2 is T, S, N, Q, M, A, V, I, L, K, E, or D; X3 is T, S, N, Q, M, A, V, I, L, K, E, or D; and X4 is T, S, N, Q, M, A, V, I, L, K, E, or D. CTPs which target prostate, pancreatic ductal, breast cancer, bone, brain, or cervical cancer cells may have a common motif of arginine, glycine, and aspartic acid, although this is not required. The CTP may target any cell surface molecule of interest including, but not limited to, a disintegrin and metalloproteinase (ADAM; e.g., ADAM15), a CXC chemokine receptor (e.g., CXCR4), epinephrine-A, an epinephrine-A receptor (e.g., EphA4); Ephrin-B3; ErbB-2; a selectin (e.g., E-selectin); Gal beta1→3GalNAc alpha disaccharide; Galectin-3; HER2 (human epidermal growth factor receptor 2); an integrin (e.g., Alpha-5 beta-3 or Alpha-5 beta-5 or Alpha-5 beta-1); Aminopeptidase N (CD13) and the integrin alpha-5 beta-3 (CD51/CD61); membrane type 1 matrix metalloproteinase (MT1-MMP); neuropilin 1 (NRP-1); Nucleolin; Podoplanin; Prospero homeobox protein 1 (Prox-1); lymphatic vessel endothelial hyaluronan receptor-1 (LYVE-1); a vascular endothelial growth factor receptor (e.g., VEGFR-10 or VEGFR-11); a proteoglycan; LDR 1; low density lipoprotein receptor (LDLR); Bombesin; or gastrin-releasing peptide.

The CPP or CTP may be a linear or a cyclic peptide. The peptide may comprise about 3-30 amino acids or about 4-20 amino acids, e.g., about 5, about 6, about 7, about 8, about 9, about 10, about 11, about 12, about 13, about 14, about 15, about 16, about 17, about 18, and about 19 amino acids, inclusive of all ranges and subranges there between. Optionally, the amino acids of the CPP or CTP belong to the 20 natural amino acids and are L-isomers. In another aspect, one or more of the amino acids belong to the 20 natural amino acids and are D-isomers. Alternatively or in addition, one or more amino acids in the peptide sequence are unnatural amino acids, including, but not limited to, protected N-methyl amino acids, L-indoline-2-carboxylic acid, L-2-cyclohexylglycine. Representative unnatural amino acid building blocks for peptide synthesis are available from commercial vendors (e.g., Chem-Impex International, Inc., Wood Dale, IL, USA).

The disclosure contemplates CPPs or CTPs having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to an amino acid sequence disclosed herein, e.g., an amino acid sequence disclosed in Table A. The disclosure also contemplates CPPs or CTPs having one, two, three, or four amino acid substitutions (e.g., conservative substitutions). Desirably, the CPP or CTP retains structural and functional features of the parent CPP or CTP. Such polypeptide variants can be prepared by substituting (using, e.g., conservative substitutions), deleting, or adding amino acid residues from the original sequences.

CPPs and CTPs are further described in, e.g., U.S. Patent Publication Nos. 20140227344 and 20220378946; U.S. patent Ser. No. 11/510,991; and (15); (16); and (17), each of which is incorporated by reference in their entireties and in particular with respect to disclosure of features of CPPs and CPP sequences. CPPs are also further described at, e.g., crdd.osdd.net/raghava/cppsite/.

### The Implantable API Delivery Device

The implantable devices disclosed herein for local or systemic API delivery comprise of the following elements:

One or more compartments that contain one or more APIs and makes up a significant portion of the device volume, also known as “kernels”,

One or more skin layers permeable to the API(s) covering one or more kernels and meet one or more of the following requirements:
a) Act as diffusion-limiting barriers to control the release of the APIs from the central compartment,
b) Protect the central compartment from one or more components of the external environment,
c) Provide structural support to the device.

The skin comprises a continuous membrane that covers all or part of the device. It is not perforated with orifices or channels that are generated during device fabrication (e.g., mechanical punching, laser drilling).

Defined microscopic pore structure. The pore structure is optionally incorporated into one, or both, of the above elements. In other words, one or more kernels and/or one or more skins have a microscopic pore structure. A “microscopic pore” structure is defined as known by those skilled in the art (18) as follows:

Microporous, with defined pores that have diameters smaller than 2 nm,

Mesoporous, with defined pores that have diameters between 2-50 nm,

Macroporous, with defined pores that have diameters larger than 50 nm and typically smaller than 250 μm.

Provided herein are drug delivery devices comprising: (a) one or more kernels comprising one or more APIs; and (b) one or more skins comprising a continuous membrane; wherein the one or more kernels and/or the skin comprises defined pores, and wherein the pores are not produced mechanically.

In some cases, the device comprises one kernel. In some cases, the device comprises a plurality of kernels.

In some cases, the kernel or kernels comprise a defined microscopic or nanoscopic pore structure. In some cases, the kernel is a reservoir kernel.

In some cases, the reservoir kernel comprises a powder comprising one or more APIs. In some cases, the reservoir kernel comprises a powder comprising one API. In some cases, the reservoir kernel comprises a powder comprising more than one APIs. In some cases, the powder comprises a microscale or nanoscale API carrier. In some cases, the powder comprises a microscale API carrier. In some cases, the powder comprises a nanoscale API carrier. In some cases, the API carrier is a bead, capsule, microgel, nanocellulose, dendrimer, or diatom.

The devices embodying these elements contain a hierarchical structure based on three levels of organization:

Primary structure: Based on the physicochemical properties of the components and materials that make up the kernel and skin of the implant. This includes, but is not limited to, elements such as polymer or elastomer composition, molecular weight, crosslinking extent, hydrophobicity/hydrophilicity, and rheological properties; API physicochemical properties such as solubility, log P, and potency.

Secondary structure: The complex microstructure of the kernel and/or the skin. This can include, but is not limited to, properties such as the API particle size, shape, and structure (e.g., core-shell architecture); fiber structures of API or excipients in kernel; pore properties (pore density, pore size, pore shape, etc.) of sponge-based kernel materials or of porous skins.

Tertiary structure: The macroscopic geometry and architecture of the implantable device. This includes elements such as, but not limited to, implant size and shape; kernel and skin dimensions (thickness, diameter, etc.); layers of kernel and/or skin and their relative orientation.

Incorporation of these elements in an implantable or injectable API-delivery device determines the characteristics of controlled, sustained delivery of one or more APIs at a predetermined location in the body (i.e., the implantation/injection site).

In one embodiment, the device is implanted or injected into a sterile anatomic compartment, including but not limited to the subcutaneous space, the intramuscular space, the eye, the ear, and the brain. In another embodiment the device is implanted into a nonsterile anatomic compartment, including but not limited to the vagina, the rectum, the oral cavity, and the nasal cavity.

The device as described herein is intended to be left in place for periods of time spanning one day to one year, or longer, and delivers one or more APIs during this period of use. In certain exemplary, non-limiting embodiments, the devices are implanted or injected subcutaneously or intramuscularly and deliver one or more APIs for 3-12 months. In certain exemplary, non-limiting embodiments, the devices are used intravaginally as IVRs and deliver one or more APIs for 1-3 months.

Additional details on exemplary embodiments are provided below.

### Implant Geometries

Implant geometries are based on multiple shapes. In one exemplary, non-limiting embodiment the shape of the device is based on a cylinder, and in some cases, the ends of the cylinder are joined to afford a toroid. These geometries are well-known in the art.

Devices for subcutaneous implantation are typically of regular, cylindrical geometry. Regular geometric shapes can simplify implant manufacture. In one embodiment, the implant has a cylindrical or rod-shaped geometry with diameter less than length, 100. Preferred lengths for rod-shaped implants are, e.g., 5-50 mm, 5-10 mm, 10-20 mm, 20-30 mm, 30-40 mm, 40-50 mm, 10 mm, 15 mm, 20 mm, 25 mm, 30 mm, 35 mm, 40 mm, 45 mm, or 50 mm. Preferred rod diameters are, e.g., 1-6 mm, 1-2 mm, 2-3 mm, 3-4 mm, 4-5 mm, 5-6 mm, 2 mm, 2.5 mm, 3 mm, 3.5 mm, 4 mm, 5 mm, or 6 mm. In an alternative embodiment, the geometry may be a rectangular prism, 102. Cylindrical or rectangular prism geometries may be flat, or may have a curved shape, 103.

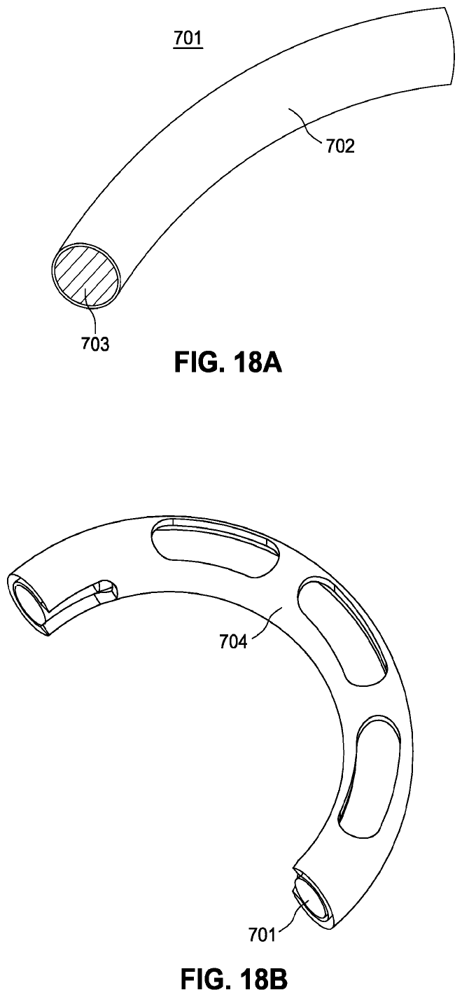

In some embodiments, the implant is shaped like a capsule, optionally from about 3 to about 50 mm in diameter and up to about 5 mm in height. In some embodiments, e.g., 600 illustrated in FIGS. 13A-13D, the implant comprises or consists of a reservoir, 602, and a non-permeable disk-shaped cover, 601 that seals the reservoir. In some embodiments, the reservoir comprises an outer sealing ring, 603, that forms a seal with the cover; one or more skin regions, 604, that are permeable to bodily fluids and API and serve as zones of API release; and none or one or more rib structures, 605, that support the skin membrane and define compartments containing a single skin region. The reservoir may be fabricated as a single part from one material, or it may be assembled from a first part comprising the outer sealing ring and any rib structures and a second part comprising a separate skin membrane that is attached to the first part using adhesive or another assembly method disclosed herein. In any of the embodiments described herein, kernels as described herein can be contained in these compartments formed between the inner reservoir surfaces and the cover. In any of the embodiments described herein, all compartments defined by the rib structures may be filled with kernel material comprising API and suitable excipients, or some compartments may be filled and some remain unfilled. In any of the embodiments described herein, all compartments contain the same kernel material. In any of the embodiments described herein different compartments may contain different kernel materials. In any of the embodiments described herein, the plurality of compartments contains a total of two kernel materials. In another preferred embodiment, the plurality of compartments contains a total of three or more kernel materials. Those skilled in the art will recognize from the disclosure provided herein that the compartments in a reservoir may contain any of a number of possible combinations of kernel materials, and all possible combinations are included herein.

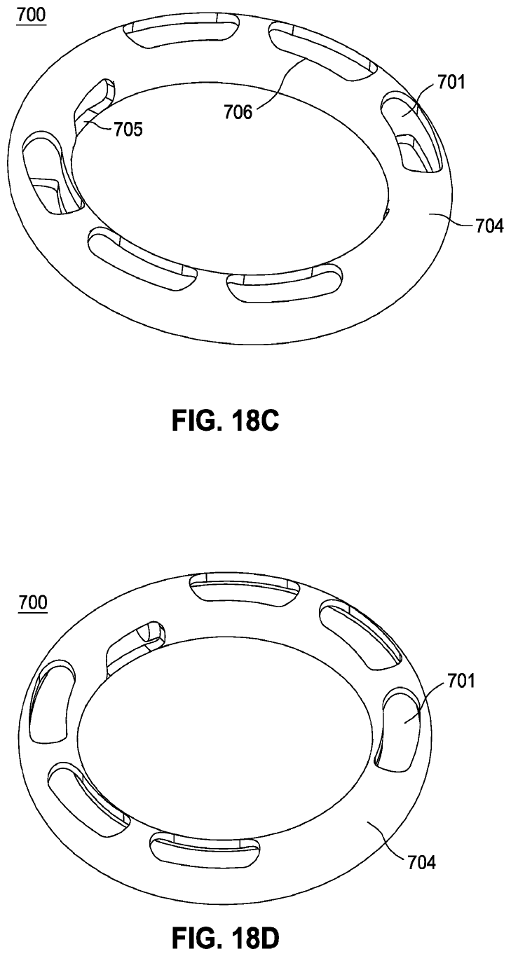

In some embodiments, e.g., those illustrated in FIGS. 14A-14G, a capsule-shaped implant comprises a skin-containing disk, 610, inserted into a API-impermeable housing, 611. In some embodiments, the housing comprises a sealing ring, 612, enclosed on one side by an impermeable backing to form a reservoir. In some embodiments, the disk (bottom view, 614, and top view, 615) comprises an outer lip, 616, that fits inside the housing's sealing ring to form a seal; one or more skin regions, 617, that are permeable to bodily fluids and API and serve as zones of API release; and none or one or more rib structures, 618, that support the skin membrane and define compartments containing a single skin region. In some embodiments, the disk may be fabricated as a single part from one material, or it may be assembled, 630, from a first part, 631, comprising the outer sealing ring and any rib structures and a second part, 632, comprising a separate skin membrane that is attached to the first part using adhesive or another assembly method disclosed herein. In some embodiments, an API is released from the one or more compartments formed between the skin membrane and housing backing, enclosed by the housing sealing ring.

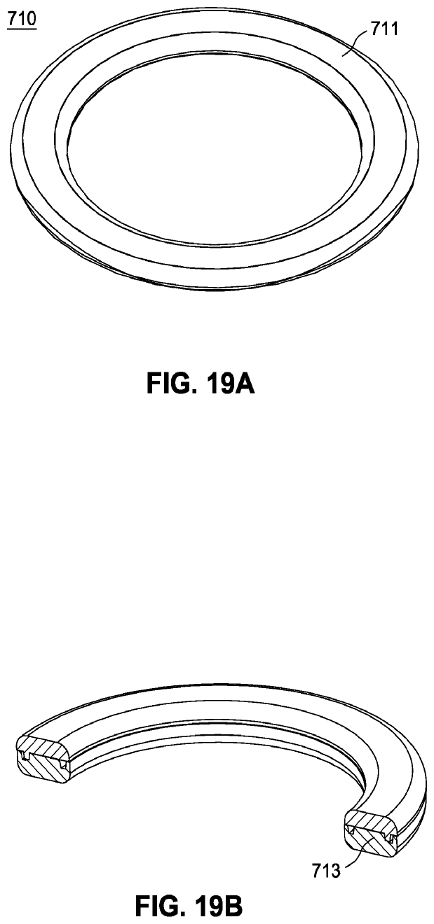

In some embodiments, e.g., those illustrated in FIGS. 15A-15E, a capsule-shaped implant, 620, comprises two skin-containing disks, 621, inserted into a API-impermeable sealing ring, 622. In some embodiments, the disks comprise an outer lip, 623, that fits inside the sealing ring to form a seal; one or more skin regions, 624, that are permeable to bodily fluids and API and serve as zones of API release; and none or one or more rib structures, 625, that support the skin membrane and define compartments containing a single skin region. In some embodiments, each disk may be fabricated as a single part from one material, or it may be assembled, 630, from a first part, 631, comprising the outer sealing ring and any rib structures and a second part, 632, comprising a separate skin membrane that is attached to the first part using adhesive or another assembly method disclosed herein. In some embodiments, an API is released from the one or more compartments formed between the two disk structures, and enclosed by the sealing ring, and any rib structures.

In some embodiments, the implant is disk-shaped with a diameter greater than or approximately equal to length, from about 3 to about 50 mm and up to about 5 mm in length.

In one, non-limiting embodiment, devices for vaginal use, such as IVRs, are toroidal in geometry, 104, with an outer diameter of 40-70 mm and a cross-sectional diameter of 2-10 mm. Preferred IVR outer diameters are 50-60 mm, or 54-56 mm and cross-sectional diameters of 3-8 mm, or 4-6 mm. The cross-sectional shape of IVRs can be other than circular, such as square, rectangular, triangular, or other shapes, 105. The IVR may contain discrete compartments containing API and other components of the API delivery function connected by sections of elastomeric material that serve to hold the compartments in a ring-like orientation and enable retention of the IVR in the vagina, 106. In another embodiment, a central compartment may contain the API delivery device, with an outer ring that functions only to retain the device in the vaginal cavity, 107. The API delivery functionality may be contained in a module that is inserted into the central compartment through an opening, 107a, with multiple large openings allowing API to exit the central compartment, but not playing a role in control of the API's release rate. In an alternate embodiment, both the ring and central compartment may contain API delivery components.

Pessaries are devices inserted into the vaginal cavity to reduce the protrusion of pelvic structures and to support and lessen the stress on the bladder and other pelvic organs. Vaginal implants for API delivery have a similar geometry to pessaries, combining vaginal API delivery with structural support. In various embodiments, a vaginal API delivery device has the geometry of a ring pessary, 110, a ring pessary with support a central structure, 111, or a Gelhorn pessary, 112. The API-releasing functionality may be contained in the ring, flat support, or knob portions of the pessaries.

In one, non-limiting embodiment, devices for vaginal use, such as IVRs, are toroidal in geometry, 104, with an outer diameter of 40-70 mm and a cross-sectional diameter of 2-10 mm. Preferred IVR outer diameters are 50-60 mm, or 54-56 mm and cross-sectional diameters of 3-8 mm, or 4-6 mm. The cross-sectional shape of IVRs can be other than circular, such as square, rectangular, triangular, or other shapes, 105. The IVR may contain discrete compartments containing API and other components of the API delivery function connected by sections of elastomeric material that serve to hold the compartments in a ring-like orientation and enable retention of the IVR in the vagina, 106. In another embodiment, a central compartment may contain the API delivery device, with an outer ring that functions only to retain the device in the vaginal cavity, 107. The API delivery functionality may be contained in a module that is inserted into the central compartment through an opening, 107a, with multiple large openings allowing API to exit the central compartment, but not playing a role in control of the API's release rate. In an alternate embodiment, both the ring and central compartment may contain API delivery components.

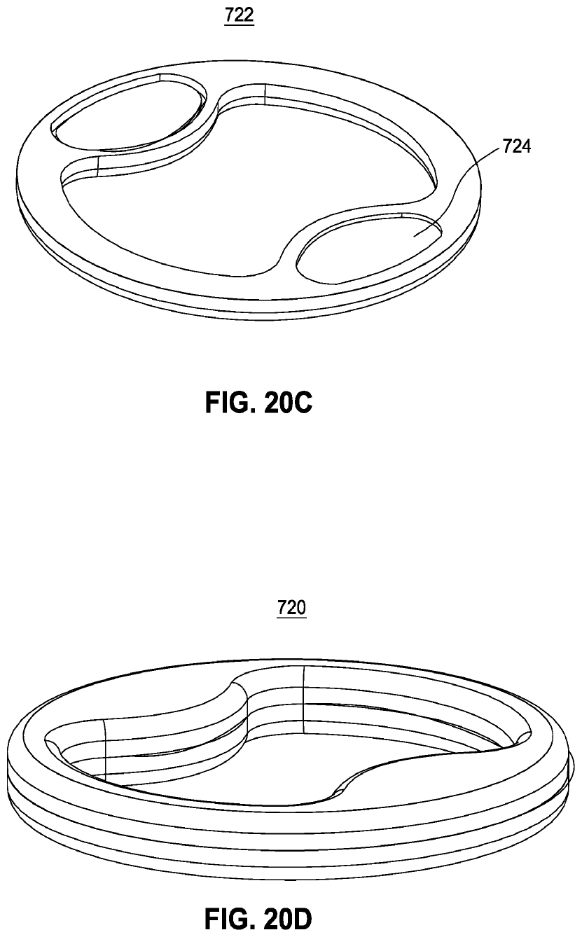

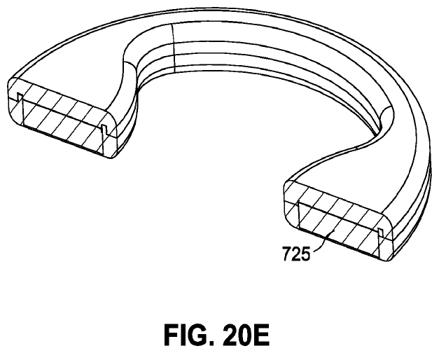

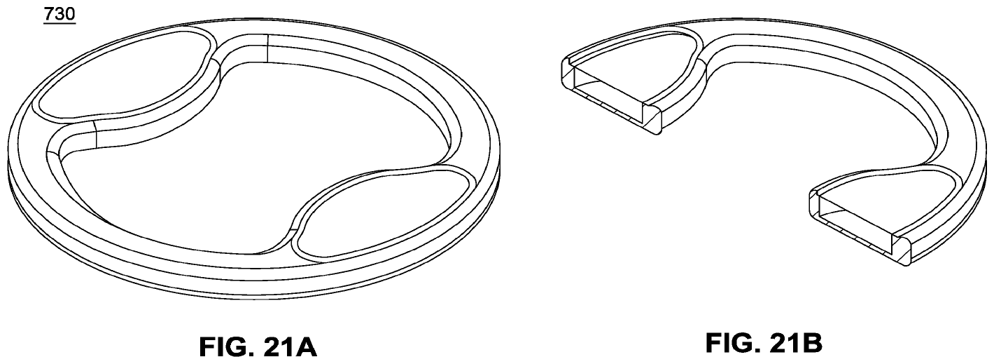

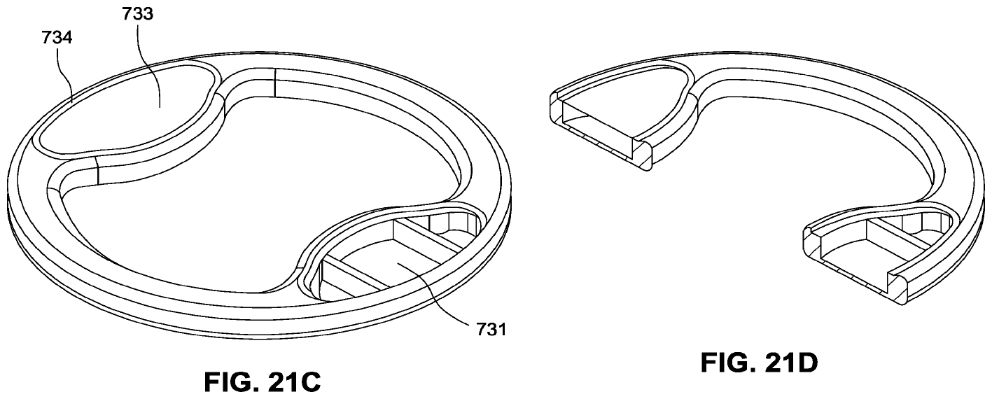

In one embodiment, e.g., 700 illustrated in FIGS. 18A-18D, a vaginal implant comprises one or more cylindrical core elements, 701, consisting of a kernel, 703, with or without a skin, 702, are held within a perforated carrier. In some cases, the skin comprises a non-medicated elastomer. Core elements are inserted into the carrier through perforations, 705. Additional perforations, 706, in the carrier allow the kernel to interact with the vaginal fluids, but perforations do not play a role in controlling the API's release rate. An alternative embodiment, e.g., 710 illustrated in FIGS. 19A-19D, comprises a molded lower structure, 712, with one or more discrete compartments comprising one or more kernels, 713. The bottom of each compartment is a drug-permeable membrane, and serves as the skin to modulate API release from the kernel. An upper structure, 711, is bonded to the carrier, 712, to seal the compartments and form a ring structure. Matching protruding and recessed structures may be located around the inner and outer circumferences of the upper and lower portions of the IVR to facilitate assembly and sealing of the device during manufacture. Alternatively, both the upper and lower structures may contain skins, allowing API release from the top and bottom surfaces of the IVR. In an alternative embodiment, e.g., 720 illustrated in FIGS. 20A-20E, compartments are contained in lobes that protrude inward from the circular outer rim of the IVR. A lower portion, 721, contains the kernel, 725, within one or more compartments, 723, of which the compartment bottom surface is API-permeable and serves as the skin. A top portion, 722, is bonded to the bottom structure, and may include matching recessed structures, 724, to facilitate sealing of the upper and lower compartment portions. Alternatively, the recessed area of the upper portion may serve as an additional API-permeable membrane to allow API release from both the upper and lower surfaces of the IVR. Another embodiment, e.g., 730 illustrated in FIGS. 21A-21E, comprises a lower structure comprising one or more compartments, 731, to contain one or more kernels. Compartments are enclosed with a discrete membrane material, 732, that is sealed to the carrier body and serves as the release rate-controlling skin. An additional protective mesh, 733, may be present on top of the skin to protect it from puncture. A sealing ring or other structure, 734, may be used to hold the skin and mesh in place on top of the kernel compartment. Compartments may contain ribs, 735, to further subdivide the compartments covered by one skin structure and to provide support to the skin and mesh.

In some cases, the device is in the shape of a torus. In some cases, the device comprises one or more cylindrical core elements disposed within a first skin, wherein the core elements comprise a kernel and optionally a second skin.

In some cases, the device comprises a molded lower structure comprising one or more compartments containing one or more kernels, and an upper structure bonded to the lower carrier to seal the plurality of compartments. In some cases, the skin covers the lower carrier. In some cases, the skin covers the lower structure and the upper structure.

In some cases, the device comprises one or more lobes protruding inward from the outer edge of the torus. In some cases, the device comprises two lobes protruding inward from the outer edge of the torus. In some cases, the one or more compartments are disposed in the lobes. In some cases, the device comprises one or more recessed structures on one part and matching protruding structures on another part to facilitate sealing of the device. In some cases, the one or more compartments comprise ribs. In some cases, the device further comprises a protective mesh disposed over the surface of the device.

An intrauterine device (IUD) is a well-established method of contraception consisting of a T-shaped implant that is placed in the uterus. Approved IUDs either deliver progestin hormone to inhibit follicular development and prevent ovulation or contain a copper wire coil that causes an inflammatory reaction that is toxic to sperm and eggs (ova), preventing pregnancy. Progestin IUDs, 120, have a central segment, 120a, that contains the progestin and copper IUDs, 121, have one or more copper wire coils, 121a, wound around the T-structure. In one embodiment, the drug delivery device is in the shape of an IUD and delivers a progestin hormone or includes one or more copper wire coils to provide contraception in addition to delivering an API for an indication other than contraception.

### The Implant Kernel

The implant kernel is the primary device component that contains API(s). Multiple, exemplary, non-limiting systems are disclosed below.

### Matrix Systems

In one embodiment, the implant kernel comprises a matrix-type design, 200. In the matrix design, the API substance(s) is(are) distributed throughout the kernel, as a solution in the elastomer, 201. In another embodiment, the API substance(s) is(are) distributed throughout the kernel in solid form as a suspension. As used herein, “solid” can include crystalline or amorphous forms. In one embodiment, the size distribution of the solid particles is polydisperse, 202. In one embodiment, the size distribution of the solid particles is monodisperse, 203. In one embodiment, the solid particles consist of nanoparticles (mean diameter<100 nm). In one embodiment, the mean diameter of the particles is between 100-500 nm. Suitable mean particle diameters can range from 0.5-50 μm, from 0.5-5 μm, from 5-50 μm, from 1-10 μm, from 10-20 μm, from 20-30 μm, from 30-40 μm and from 40-50 μm. Other suitable mean particle diameters can range from 50-500 μm, from 50-100 μm, from 100-200 μm, from 200-300 μm, from 300-400 μm, and from 400-500 μm. Suitable particle shapes include spheres, needles, rhomboids, cubes, and irregular shapes, for example.

In one embodiment, the implant core comprises or comprises a plurality of modular kernels assembled into a single device, and each module is a matrix type component containing one or more API substances. In one embodiment, the modules can be joined directly to one another (e.g., ultrasonic welding), 204 or separated by an impermeable barrier to prevent API diffusion between segments, 205.

At least part of the matrix-type devices disclosed herein are covered with one or more skins, as described more fully under “The Implant Skin”.

### Reservoir Systems

In one embodiment, the implant comprises a reservoir-type design, 206. In the reservoir implant, one or more kernels, 206a, are loaded with the API substance(s). The kernel can span the entire length of the device, or a partial length. The kernel is partially or completely surrounded by a skin, 206b, (described in more detail under “The Implant Skin”) that, in some embodiments, forms a barrier to API diffusion; i.e., slows down the rate of API release from the device. Accordingly, the release of API substances from such implants is dependent upon permeation (i.e., molecular dissolution and subsequent diffusion) of the kernel-loaded API substance through the outer sheath, or skin. API release rates can be modified by changing the thickness of the rate-controlling skin, as well as the composition of the skin. The API release kinetics from reservoir type implants are zero to first order, depending on the characteristics of the kernel and skin.

There are many embodiments describing the physical and chemical characteristics of the reservoir kernel. In one embodiment, the kernel comprises a powder made up of the API with or without excipients.

In another embodiment, the powder making up the reservoir kernel comprises microscale (1-1,000 μm cross-section) or nanoscale (1-1,000 nm cross-section) API carriers. The API carriers are particulate materials containing the API, either internally or on the surface. Non-limiting examples of such carriers, known in the art, are beads; capsules; microgels, including but not limited to chitosan microgels (19); nanocelluloses (20, 21); dendrimers; and diatoms (22, 23), included herein by reference. The carriers are filled or coated with API using impregnation or other methods known in the art (e.g., lyophilization, rotary solvent evaporation, spray-drying).

In another embodiment, the kernel comprises one or more pellets or microtablets, 207 (24). In these embodiments, it may be desirable to maximize the API loading and to minimize the use of excipients. However, the use of excipients can lead to beneficial physical properties such as lubrication and binding during tableting.

Provided herein are devices comprising a kernel comprising a pellet, a tablet, or a microtablet. In some cases, the kernel comprises a pellet. In some cases, the kernel comprises a tablet. In some cases, the kernel comprises a microtablet.

### Semisolid Preparations (Pastes)

In one non-limiting embodiment of the disclosure, the kernel comprises solid API particles blended or mixed with one or more liquid, or gel, excipients to form a semisolid preparation, or paste. This embodiment holds the advantage of making the formulation easily dispensable into the implant shell, leading to manufacturing benefits. The nature of the excipient also can affect the API release kinetics from the preparation. The paste is contained in a shell or structure such as a tube or cassette. The paste can be separated from the exterior environment by one or more skins, as described herein. In certain embodiments, the structure can act as a skin. Non-limiting examples of structures that surround and contain the kernel paste include, but are not-limited to tubes or cartridges. In certain embodiments, the structures are made up of solid/continuous (non-porous) elastomers, both non-resorbable—e.g., silicone, ethylene vinyl acetate (EVA), and poly(urethanes) as described herein- and resorbable—e.g., poly(caprolactones) (PCLs) as described herein. In certain embodiments, the structures are made up of porous materials—e.g., expanded poly(tetrafluoroethylene) (ePTFE) and porous metals as described herein.

In one embodiment, the liquid excipient comprises an oil with a history of pharmaceutical use, including subcutaneous or intramuscular use. Non-limiting examples of such oils known in the art include: triethyl citrate (TEC), polyethylene glycol (PEG; e.g., PEG-300 and PEG-400), and vegetable oils (e.g., sunflower oil, castor oil, sesame oil, etc.). The paste may comprise API particles and a single liquid, or it may be a mixture of two or more liquids with API particles. In some embodiments, one or more additional excipients may be added to the paste to modify selected paste properties, including physical properties (e.g. viscosity, adhesion, lubricity) and chemical properties (e.g. pH, ionic strength). In some cases, the use of excipients can affect the solubility, and hence implant release rate, of the API substance from the kernel. Certain excipients can be used to increase the solubility of APIs in water, and others can decrease the solubility. In some cases, excipients can lead to API stabilization. Exemplary excipients are described in more detail below (see “API Formulation”). In another embodiment, pastes as described above may contain a blend of more than one API for the purpose of delivering two or more API substances from a single kernel.

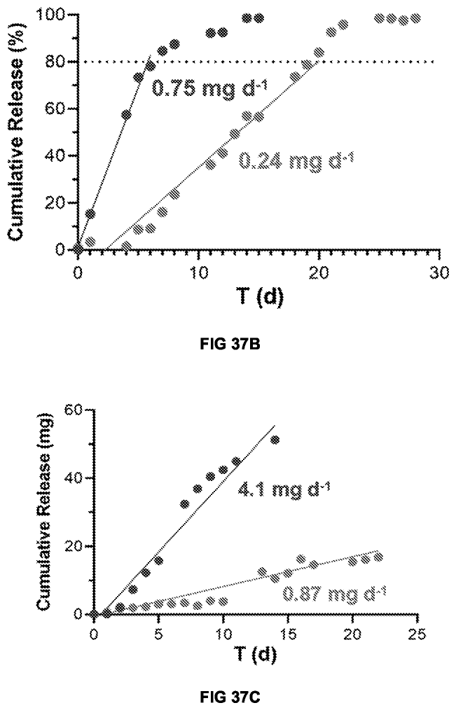

In another embodiment, the excipient comprises a so-called “ionic liquid” (25-27), incorporated by reference in their entirety. Broadly defined as salts that melt below 100° C. and composed solely of ions, ionic liquids are well-known in the art. The choice of cation strongly impacts the properties of the ionic liquid and often defines its stability. The chemistry and functionality of the ionic liquid generally is controlled by the choice of the anion. In one embodiment, the concentration of API substance particles in the paste is 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w. FIG. 37C shows illustrative in vitro results of how different excipients making up the paste can affect the release kinetics of, e.g., the biomolecule hIgG, through ePTFE tubes.

In one nonlimiting set of embodiments, the paste comprises a phase inversion system, wherein a semisolid API paste undergoes a phase inversion when contacted with physiological fluids, such as subcutaneous, cervicovaginal, and oral fluids. The phase inversion results in hardening of the kernel to produce a solid or semi-solid structure in situ. In one nonlimiting embodiment, the phase inversion system comprises a resorbable polymer [e.g., poly(lactic acids), poly(glycolic acids), poly(lactic-co-glycolic acids), poly(caprolactones), and mixtures thereof] and a pharmaceutically acceptable, water-miscible solvent (e.g., N-methyl-2-pyrrolidone, 2-pyrrolidone, ethanol, propylene glycol, acetone, benzyl alcohol, benzyl benzoate, methyl acetate, ethyl acetate, methyl ethyl ketone, dimethylformamide, dimethyl sulfoxide, tetrahydrofuran, caprolactam, decylmethyl sulfoxide, and the like). Such formulations are suitable for subcutaneous injection, sometimes referenced as “in situforming implants”. See, for example, Dunn et al. (28-31), incorporated by reference in their entirety. In one embodiment, the concentration of API substance particles in the paste is 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w.

Phase transition systems that are based on phospholipids alone or in combination with medium chain triglycerides and a pharmaceutically acceptable, water-miscible solvent (vide supra) also are known in the art to form solid or semi-solid depots when in contact with physiological fluids and are used in the disclosed invention to make up the kernel. In some embodiments, the phase inversion system comprises one or more phospholipids. In some cases, the phase inversion system comprises a combination of one or more phospholipids and one or more medium-chain triglycerides (MCTs). Illustrative examples that are incorporated by reference in their entirety include (32-36). In non-limiting embodiments, the phospholipids are animal-based (e.g., derived from eggs), plant-based (e.g., derived from soy), or synthetic. Commercial suppliers of phospholipids include, but are not limited to, Creative Enzymes, Lipoid, and Avanti. In one non-limiting embodiment, the phospholipid is lecithin. In some embodiments, the MCT comprises triglycerides from a range of carboxylic acids, for example and without limitation, those supplied by ABITEC Corporation. In one embodiment, the concentration of API substance particles in the paste is, e.g., 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w.

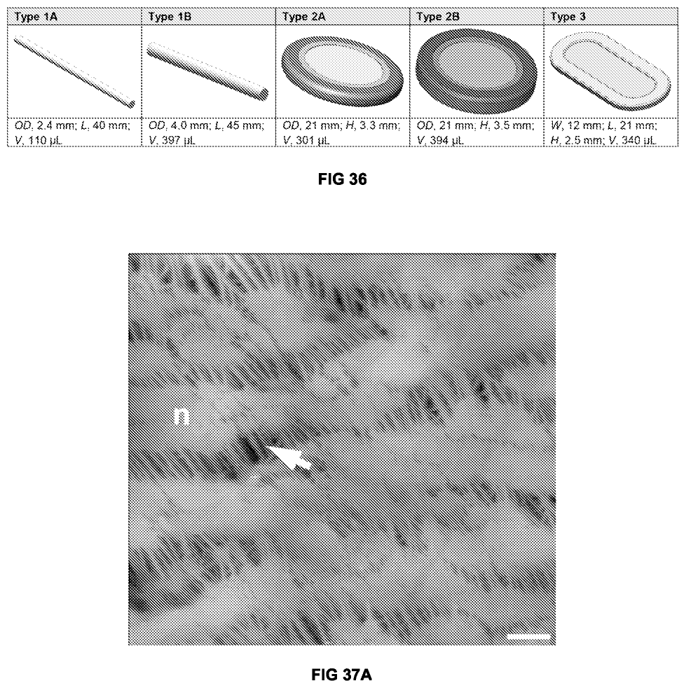

In some embodiments, the phase inversion system comprises one or more lyotropic liquid crystals. In another, non-limiting set of embodiments, the excipient formulation making up the kernel paste-API suspension leads to a lyotropic liquid crystal when in contact with physiological fluids. Certain lipid-based systems, such as monoglycerides, including but not limited to compounds 1-5 below, form lyotropic liquid crystal in the presence of water (37). These systems self-assemble into ordered mesophases that contain nanoscale water channels, while the rest of the three-dimensional structure is hydrophobic. FIG. 36 shows illustrative XRD spectra of monoolein (MYVEROL 18-92K, food emulsifier) mixed with 20% and 30% w/w water to self-assemble into a network of ordered channels ca. 5 nm wide.

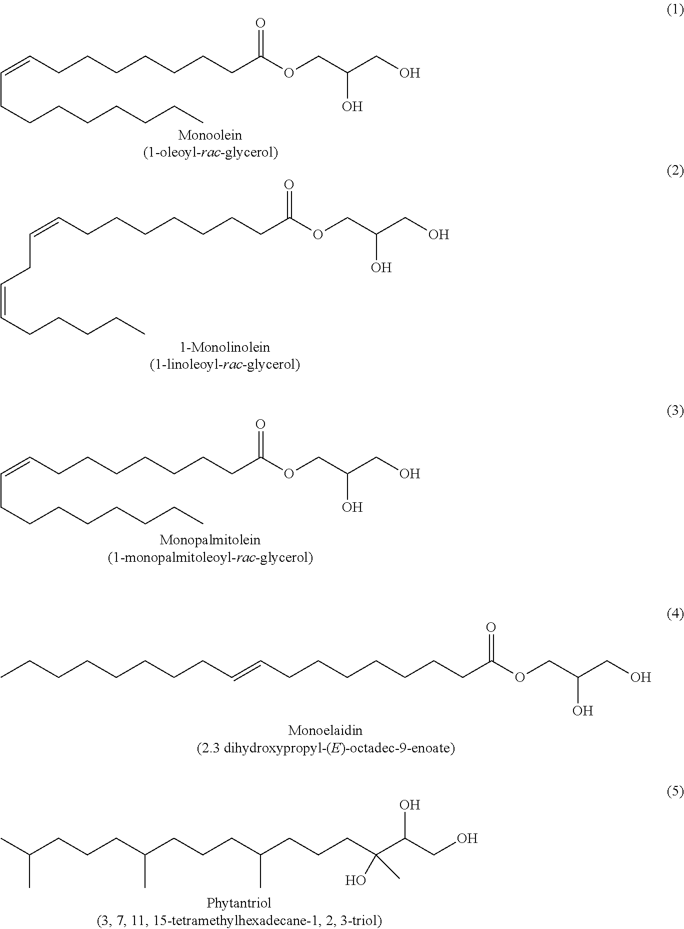 [SMILES: CCCCCCCC/C=C\CCCCCCCC(=O)OCC(O)CO ; CCCCC/C=C\C/C=C\CCCCCCCC(=O)OCC(O)CO ; CCCCCCCC/C=C\CCCCCCCC(=O)OCC(O)CO ; CCCCCCCC/C=C/CCCCCCCC(=O)OCC(O)CO ; CC(C)CCCC(C)CCCC(C)CCCC(C)(O)C(O)CO]

In one embodiment, lyotropic lipid-based systems can be used to form paste formulation suspensions with API substance particles. In one embodiment, the concentration of API substance particles in the paste is, e.g., 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w.

In another, non-limiting embodiment, the paste comprises shape-memory self-healing gels, as known in the art. Illustrative examples that are incorporated by reference in their entirety include (38-40). Shape retaining injectable hydrogels based on a polysaccharide backbone (e.g., alginate, chitosan, HPMC, hyaluronic acid) and, in some cases, non-covalently crosslinked with nanoparticles (unmedicated or medicated) form part of this embodiment for semisolid preparations, including (41-43) incorporated by reference in their entirety. In one embodiment, the physically crosslinking nanoparticles comprise or consist of API nanoparticles. In one embodiment, the concentration of API substance particles in the paste is 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w.

In one embodiment of the disclosure, the paste comprises a stimulus-responsive gel, described in (44, 45), incorporated by reference in their entirety. Such gels change their physical properties (e.g., liquid to viscous gel or solid) in response to external or internal stimuli, including, but not limited to temperature (46), pH, mechanical (i.e., thixotropic), electric, electrochemical, magnetic, electromagnetic (i.e., light), and ionic strength. In one non-limiting embodiment of thermosensitive polymers suitable for kernel formulation consist of amphiphilic tri-block copolymers of poly(ethylene oxide) and poly(propylene oxide) (PEO-PPO-PEO), including linear (e.g., poloxamers or Pluronic®) or X-shaped (e.g., poloxamines or Tetronic®). This group of polymers is suitable for API delivery; see, e.g., (47), incorporated by reference in its entirety. In one embodiment, the concentration of API substance particles in the paste is 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w.

Provided herein are devices comprising a paste comprising one or more APIs. In some cases, the device comprises one or more reservoir kernels comprising a paste comprising one or more APIs. In some cases, the paste comprises an oil excipient, an ionic liquid, a phase inversion system, or a gel. In some cases, the paste comprises an oil excipient. In some cases, the paste comprises an ionic liquid. In some cases, the paste comprises a phase inversion system. In some cases, the paste comprises a gel.

In some cases, the phase inversion system comprises a biodegradable polymer, a combination of phospholipids and medium-chain triglycerides, or lyotropic liquid crystals. In some cases, the phase inversion system comprises a biodegradable polymer. In some cases, the phase inversion system comprises a combination of phospholipids and medium-chain triglycerides. In some cases, the phase inversion system comprises lyotropic liquid crystals.

In some cases, the gel is a stimulus-responsive gel or a self-healing gel. In some cases, the gel is a stimulus-responsive gel. In some cases, the gel is a self-healing gel.

In some embodiments, multiple reservoir modules (208a, 208b) are joined to form a single implant, 208. In some embodiments, 209, the segments are separated by an impermeable barrier, 209a, to prevent API diffusion between segments.

### Fiber-Based Systems

In another embodiment, the API kernel may comprise or consist of API dispersions in high surface area fiber-based carriers, which are suitable for tissue engineering, delivery of chemotherapeutic agents, and wound management devices, as described in (48), included herein by reference in its entirety. In one embodiment, the high surface area carrier comprises fibers produced by electrospraying. In one embodiment, the high surface area carrier comprises electrospun fibers, including, but not limited to electrospun nanofibers. Electrospun fibers are further described in, for example (49-56), incorporated by reference in their entirety.

Electrospun, API-containing fibers can have a number of configurations. For example, in one embodiment, the API is embedded in the fiber (57), a miniaturized version of the above matrix system. In another exemplary embodiment, the API-fiber system is produced by coaxial electrospinning to give a core-shell structure (58, 59), a miniaturized version of the above reservoir system. Core-shell fibers production by coaxial electrospinning produces encapsulation of water-soluble agents, such as biomolecules including, but not limited to proteins, peptides, and the like (60). In yet another exemplary embodiment, Janus nanofibers can be prepared; exemplary suitable methods are described in (61). Janus fibers contain two or more separate surfaces having distinct physical or chemical properties, the simplest case being two fibers joined along an edge coaxially. In some embodiments, it may be advantageous to modify the fibers by surface-functionalization, as described in, e.g., (62, 63), included herein by reference in its entirety.

At least part of the fiber-based devices disclosed herein are covered with one or more skins, as described more fully under “The Implant Skin”.

Electrospun fibers may be used to form the kernel of a reservoir implant. In one embodiment, a reservoir implant is formed by packing API-containing fibers into a tubular implant skin and sealing the tube ends as described in a subsequent section. Fibers formed by electrospinning may be collected on a plate or other flat surface and chopped, ground, or otherwise reduced in size by methods known in the art to a size that can be effectively packed into the implant, forming a packed powder kernel. The resulting reduced-sized electrospun fiber material may also be formulated into a kernel using any of the methods described herein for API powder or API-excipient powder mixtures. In an alternative embodiment, the electrospun fibers may be collected on a fixed or stationary collector surface (e.g., a plate or drum) in the form of a mat. The mat may be subsequently cut to an appropriate size and geometry (e.g., cut into strips or sheets), and placed in a tubular skin structure to form a reservoir implant. In another embodiment, the electrospun, API-containing mat may be rolled into a multi-layer cylindrical shape to form the kernel of a tubular reservoir implant. In yet another embodiment, the kernel is formed from an electrospun fiber yarn fabricated; suitable methods are described in, e.g., (64-68), included herein by reference in their entirety. In another embodiment, an electrospun fiber kernel in a cylindrical geometry may be prepared by collecting fibers during the spinning process directly on a rotating wire, fiber, or small diameter mandrel.

Electrospinning may also be used to create skins. In one embodiment, a membrane or mat of electrospun fibers collected on a rotating plate or drum may be used as a skin. Skins formed in this fashion may be wrapped around a pre-formed kernel to form a reservoir implant, or may be rolled into a tubular shape and be filled with a kernel material and sealed. Alternatively, a tubular skin may be formed directly by collecting electrospun fibers on a rotating mandrel during the spinning process.

An alternative embodiment utilizes electrospinning processes to fabricate both the kernel and skin, using the methods described herein for each. In yet another embodiment, electrospinning may be used to form the skin layer, kernel layer, or both in layered implant embodiments described in a subsequent section.

The above paragraphs describe embodiments incorporating fibers produced by electrospinning, but additional, non-limiting embodiments use the same approaches incorporating fibers formed by alternative spinning methods. In one embodiment, rotary jet spinning, a perforated reservoir rotating at high speed propels a jet of liquid material outward from the reservoir orifice(s) toward a stationary cylindrical collector surface. The fiber material may be liquefied thermally by melting, resulting in a process analogous to that used in a cotton candy machine, or dissolved in a solvent to allow fiber production at low temperature (i.e., without melting the material). Prior to impaction, the jet stretches, dries, and eventually solidifies to form nanoscale fibers in a mat or bundle on the collector surface. The fiber material can consist of a pharmaceutically acceptable excipient, such as glucose or sucrose, or a polymer material e.g., a resorbable or non-resorbable polymer described herein. In another embodiment, the solid API and excipient(s) or polymer are premixed as solids and formed into a fiber mat by spinning. Rotary jet spinning methods are known in the art, for example (69-72), incorporated by reference in their entirety.

In another embodiment, fibers may be produced by wet spinning (73) or dry-jet wet-spinning (74, 75) methods. In wet spinning, fibers are formed by extrusion of a polymer solution from a small needle spinneret into a stationary or rotating coagulating bath consisting of a solvent with low polymer solubility, but miscibility with the polymer solution solvent. Dry-jet wet-spinning is a similar process, with initial fiber formation in air prior to collection in the coagulation bath.

Provided herein are devices wherein the kernel comprises a fiber-based carrier. In some cases, the fiber-based carrier comprises an electrospun microfiber or nanofiber. In some cases, the fiber-based carrier comprises an electrospun microfiber. In some cases, the fiber-based carrier comprises an electrospun nanofiber. In some cases, the electrospun nanofiber is a Janus microfiber or nanofiber. In some cases, the electrospun nanofiber is a Janus microfiber. In some cases, the electrospun nanofiber is a Janus nanofiber.

In some cases, the fiber-based carrier comprises random or oriented fibers. In some cases, the fiber-based carrier comprises random fibers. In some cases, the fiber-based carrier comprises oriented fibers.

In some cases, the fiber-based carrier comprises bundles, yarns, woven mats, or non-woven mats of fibers. In some cases, the fiber-based carrier comprises bundles, yarns, woven mats, or non-woven mats of fibers. In some cases, the fiber-based carrier comprises bundles of fibers. In some cases, the fiber-based carrier comprises yarns of fibers. In some cases, the fiber-based carrier comprises woven mats of fibers. In some cases, the fiber-based carrier comprises non-woven mats of fibers.

In some cases, the fiber-based carrier comprises rotary jet spun, wet spun, or dry-jet spun fibers. In some cases, the fiber-based carrier comprises rotary jet spun fibers. In some cases, the fiber-based carrier comprises wet spun fibers. In some cases, the fiber-based carrier comprises dry-jet spun fibers.

In some cases, the fiber comprises glucose, sucrose, or a polymer material. In some cases, the fiber comprises glucose. In some cases, the fiber comprises sucrose. In some cases, the fiber comprises a polymer material. In some cases, the polymer material comprises a resorbable or non-resorbable polymer material described herein, e.g., poly(dimethyl siloxane), silicone, a poly(ether), poly(acrylate), poly(methacrylate), poly(vinyl pyrolidone), poly(vinyl acetate), poly(urethane), cellulose, cellulose acetate, poly(siloxane), poly(ethylene), poly(tetrafluoroethylene) and other fluorinated polymers, poly(siloxanes), copolymers thereof, or combinations thereof. In some cases, the polymer comprises expanded poly(tetrafluoroethylene) (ePTFE) or ethylene vinyl acetate (EVA). In some cases, the polymer comprises expanded poly(tetrafluoroethylene) (ePTFE). In some cases, the polymer is ethylene vinyl acetate (EVA). In some cases, the polymer comprises poly(amides), poly(esters), poly(ester amides), poly(anhydrides), poly(orthoesters), polyphosphazenes, pseudo poly(amino acids), poly(glycerol-sebacate), poly(lactic acids), poly(glycolic acids), poly(lactic-co-glycolic acids), poly(caprolactones) (PCLs), PCL derivatives, amino alcohol-based poly(ester amides) (PEA), poly(octane-diol citrate) (POC), copolymers thereof, or mixtures thereof.

### Porous Sponge Systems

In some embodiments, the implant kernel comprises a porous support structure containing the API. The support has a porous microstructure (pore sizes 1-1,000 μm). In some embodiments, the support has a porous nanostructure (pore sizes 1-1,000 nm). In yet other embodiments, the support has both porous microstructures and nanostructure. Examples of these microscopic pores include, but are not limited to sponges, including: silica sol-gel materials (76); xerogels (77); mesoporous silicas (78); polymeric microsponges (79); including polydimethylsiloxane (PMDS) sponges (80, 81) and polyurethane foams (82); nanosponges, including cross-linked cyclodextrins (83); and electrospun nanofiber sponges (84) and aerogels (85), all incorporated herein by reference. In some embodiments, the porous sponge comprises silicone, a silica sol-gel material, xerogel, mesoporous silica, polymeric microsponge, polyurethane foam, nanosponge, or aerogel. In some embodiments, the porous sponge comprises silicone. In some embodiments, the porous sponge comprises a silica sol-gel material, xerogel, mesoporous silica, polymeric microsponge, polyurethane foam, nanosponge, or aerogel.

In other embodiments, the implant kernel comprises a porous metal structure. Porous metallic materials including, but not limited to, titanium and nickel-titanium (NiTi or Nitinol) alloys in structural forms including foams, tubes, and rods, may be applied as both kernel and skin materials. Such materials have been used in other applications including bone replacement materials (86-88), filter media (89, 90), and as structural components in aviation and aeronautics (91). These materials have desirable properties for API delivery devices including resistance to corrosion, low weight, and relatively high mechanical strength. Importantly, these properties can be controlled by modifying pore structure and morphology. The pore architecture can be uniform, bimodal, gradient, or honeycomb, and the pores can be open or closed. NiTi alloys additionally have shape-memory properties (ability to recover their original shape from a significant and seemingly plastic deformation when a particular stimulus, such as heat, is applied) and superelastic properties (alloy deforms reversibly by formation of a stress-induced phase under load that becomes unstable and regains its original phase and shape when the load is removed). For NiTi alloys, these properties are due to transformation between the low-temperature monoclinic allotrope (martensite phase) and high-temperature cubic (austenite) phase. Porous NiTi materials maintain shape memory and/or superelastic properties (92). Both mechanical properties and corrosion resistance are determined by the chemical composition of the titanium alloy. Surface treatment, including chemical treatment, plasma etching, and heat treatment, may be employed to increase or decrease the bioactivity of Ti and Ti-alloy porous materials. Porous Ti metal with 40% in porosity and 300-500 μm pore size was penetrated with newly grown bone more deeply following NaOH and heat treatments (93).

There are few examples of API-loaded nanoporous coatings on implants or implantable devices that have been used to deliver agents in a sustained fashion, such as in (94), incorporated herein in full by reference. In a rare example, antibiotic-loaded layered double hydroxide coatings on porous titanium metal substrates have been shown to limit infection for over 1 week (95). In these cases, API release is directly from the thin coating (analogous to API-releasing stents), not from the bulk implant material (porous or solid), and these systems typically exhibit first-order dissolution kinetics.

In one embodiment, the implant kernel comprises sponge structure known in the art—illustrative examples are provided above- and the API is incorporated by impregnation using methods known in the art. In one non-limiting example, the API is introduced into the inner sponge microarchitecture using a liquid medium that has an affinity for the sponge material. For example, polydimethylsiloxane (PDMS) is a material commonly used in the art that is highly hydrophobic. A PDMS sponge therefore can be readily impregnated with a nonpolar solvent solution of the API, followed by drying. Multiple impregnation cycles allow for API accumulation in the device. In another non-limiting embodiment, the solvent acts as a vehicle to load a API particle suspension into the sponge. In a related embodiment, a biomolecule (e.g., peptide or protein) is suspended in n-hexane and impregnated into a PDMS sponge followed by room temperature drying in a vacuum oven. Multiple impregnation-drying cycles are used to increase API loading. In a non-limiting example, a suspension of VRC01, a broadly neutralizing antibody against HIV, in n-hexane, is impregnated into a PDMS sponge. In another non-limiting example, a suspension of tenofovir alafenamide, in n-hexane, is impregnated into a PDMS sponge. The above examples are illustrative of API impregnation of PDMS sponges, including with peptide-drug conjugates.

In some embodiments, the sponges are magnetic to enable, for example, remotely triggered API release. See, e.g., (96), incorporated herein by reference.

In one embodiment, the sponge pores are created in situ during use using a templating excipient. A number or porogens are known in the art and have been used to generate porous structures, such as described in (97), incorporated by reference herein in its entirety. Methods for creating pores during use (i.e., in vivo) include, but are not limited to, the inclusion of excipient particles in implant kernels that dissolve when exposed to bodily fluids, such as subcutaneous fluid and cervicovaginal fluid. As used herein, solid particles can include crystalline or amorphous forms. In one embodiment, the size distribution of the solid particles is polydisperse. In one embodiment, the size distribution of the solid particles is monodisperse. In one embodiment, the solid particles comprise or consist of nanoparticles (mean diameter<100 nm). In one embodiment, the mean diameter of the particles can range from 1-10 nm, 10-25 nm, 25-100 nm, and 100-500 nm. Suitable mean microparticle diameters can range from 0.5-50 μm, from 0.5-5 μm, from 5-50 μm, from 1-10 μm, from 10-20 μm, from 20-30 μm, from 30-40 μm and from 40-50 μm. Other suitable mean particle diameters can range from 50-500 μm, from 50-100 μm, from 100-200 μm, from 200-300 μm, from 300-400 μm, from 400-500 μm, and from 0.5-5 mm. Suitable particle shapes include spheres, needles, rhomboids, cubes, and irregular shapes. Said templating particles can consist of salts (e.g., sodium chloride), sugars (e.g., glucose), or other water-soluble excipients known in the art. One skilled in the art would know how to produce such particles of well-defined shape and size. The mass ratio of pore-forming particles to API in the kernel ranges from 100 to 0.01. More specifically, said ratio can range from 100-20, from 20-5, or from 5-1. In other embodiments, the ratio can range from 1-0.2, from 0.2-0.05, or from 0.05-0.01.

In one non-limiting embodiment, the porogen comprises a fiber mat, as described above. In another embodiment, the porogen comprises a mat of microfibers. In another embodiment, the porogen comprises a mat of nanofibers. The fiber mat is fabricated by any suitable methods, such as those known in the art. In one embodiment, the fibers are produced by electrospinning. In another embodiment, the fibers are produced by rotary-jet spinning. In yet another embodiment, the fibers are produced by wet-jet spinning or dry-jet wet-spinning. The fiber material can comprise or consist of one or more biocompatible polymers (resorbable and non-resorbable) as listed herein. The fiber material can also comprise or consist of a pharmaceutically acceptable excipient, such as glucose (i.e., cotton candy).

In one non-limiting embodiment, the porogen particles are fused by exposure to suitable solvent vapors. Particle fusion can be required to result in an open-cell sponge architecture that may be desirable. A non-limiting example of porogen particle fusion is provided in Example 11. The fusing solvent can be a polar solvent such as water or an organic solvent with polarities ranging from polar (e.g., methanol) to nonpolar (e.g., hexane), depending on the solubility of the templating agent. The solvent vapors are generated by any suitable method, such as heating, with the column of porogen particles suspended in contact with the vapors using a screen, mesh, or perforated plate, or a suitable container, such as a Buchner funnel, with or without a filter. The exposure time can be determined experimentally to achieve the desired degree of particle fusion.

In some embodiments, the pores are formed during manufacture (i.e., prior to use) by immersing the device in a suitable fluid (e.g., water or organic solvent) to dissolve the porogens.

In some embodiments, the pores can form as a result of mechanical, temperature, or pH changes following implantation/use.

In one non-limiting embodiment, one or more APIs make up the sponge templating agent(s). As the agent(s) are released from the device, the sponge is formed. In one embodiment, the API templating agent comprises a mat of microneedles. In a non-limiting example, the API templating agent comprises a mat of tenofovir alafenamide microneedle crystals as described in Example 4.

In one non-limiting embodiment, the sponge is made up of PDMS and the hydrophobic microscopic channels are modified using methods known in the art, such as chemical and plasma treatment. In another embodiment, a linking agent is used between the internal PDMS microchannels and a surface modifying agent to tailor the internal surface properties of the sponge. The surface modifying chemistry is well-known in the art. In one, non-limiting embodiment 3-aminopropyl)triethoxysilane is used as the linking agent and a protein is attached to the PDMS surface as described by Priyadarshani et al. (98), incorporated by reference herein in its entirety.

Provided herein are devices wherein the kernel comprises a porous sponge. In some cases, the porous sponge comprises silicone, a silica sol-gel material, xerogel, mesoporous silica, polymeric microsponge, polyurethane foam, nanosponge, or aerogel. In some cases, the porous sponge comprises silicone. In some cases, the porous sponge comprises a silica sol-gel material. In some cases, the porous sponge comprises xerogel. In some cases, the porous sponge comprises mesoporous silica. In some cases, the porous sponge comprises polymeric microsponge. In some cases, the porous sponge comprises polyurethane foam. In some cases, the porous sponge comprises nanosponge. In some cases, the porous sponge comprises aerogel.

In some cases, the porous sponge comprises a porogen. In some cases, the porogen comprises a fiber mat. In some cases, the fiber mat comprises glucose. In some cases, the porogen comprises an API. In some cases, the porous sponge is impregnated with the API. In some cases, the porous sponge comprises a sponge material that has an affinity for a solvent capable of dissolving an API. In some cases, the porous sponge comprises polydimethylsiloxane (PDMS).

At least part of the porous devices disclosed herein are covered with one or more skins, as described more fully under “The Implant Skin”.

### The Implant Skin

It is advantageous to have a skin as part of the disclosed devices, which can cover the kernel partially or in its entirety.

The in vitro and in vivo API release profile of the matrix implants disclosed herein generally are non-linear, with an initial burst of API release followed by a low, sustained release phase. In certain indications, it may be desirable to linearize the API release properties of the implant. In an advantageous embodiment of such an implant, 300, the external surface of the device, 301, is covered by a rate-controlling skin, 302. In one embodiment, the skin is made up of a biocompatible elastomer, as described here. The composition and thickness of the skin determines the extent of linearization of the API release as well as the rate of API release. The skin thickness can range from, e.g., 5-700 μm. Suitable thicknesses of the skin can range from 5-700 μm, from 10-500 μm, from 15-450 μm, from 20-450 μm, from 30-400 μm, from 35-350 μm, and from 40-300 μm.

In certain embodiments the thickness of the skin is 10 μm, 20 μm, 30 μm, 40 μm, 50 μm, 60 μm, 70 μm, 80 μm, 90 μm, 100 μm, 125 μm, 150 μm, 175 μm, 200 μm, 225 μm, 250 μm, and 300 μm. In some embodiments, the thickness of the skin is 30 μm, 50 μm or 80 μm. These skin characteristics also apply to reservoir-type designs.

In one series of embodiments, a single external skin encases the API-containing compartment. In another embodiment, 303, a plurality of external skins encases the API-containing compartment. In some embodiments 2-20 independent (303b,c), layered skins encase the API-containing compartment, 303a. In some embodiments, these skins comprise or consist of the same material, with the same or different thicknesses. In some embodiments, these skins comprise or consist of one or more different materials, with the same or different thicknesses.

In another series of embodiments, a plurality of skins is distributed throughout the device isolating different regions of the main component volume from each other. In one embodiment one can envision these interspersed skins by analogy to the rings in a tree trunk, 304. The skins (304b, 304d) in such embodiments can consist of one or more different materials, with the same or different thicknesses. The volumes (kernels—304a, 304c) separated by the skins can all contain the same API at the same concentration, or different APIs at different concentrations. Some of said volumes may be unmedicated. Excipients in or making up said volumes can be the same or different across compartments separated by the skins.

In certain embodiments described herein, the implant kernel can be a single compartment. In other embodiments, the kernel of the drug delivery devices described herein may comprise two compartments in a segmented arrangement as in 208 or arranged in two layers (401, 402) as in 400. In other embodiments, the kernel of the drug delivery devices described herein may comprise more than two compartments or layers. Each kernel layer may contain one or more therapeutic agents, or no therapeutic agents. For example, in certain embodiments of the implant drug delivery devices described herein, the kernel comprises a first layer, 401, and a second layer, 402, wherein the second layer is adjacent to the skin, 403, and the first layer is adjacent to the second layer. A second skin layer, 404, may optionally be present adjacent to the first skin layer. In certain embodiments, one or more skin layers may contain a therapeutic agent as described previously for embodiments 300, 303, and 304. In one embodiment, the first kernel layer, 401, is completely surrounded by the second kernel layer, 402. Only the second kernel layer is in contact with the first skin layer. In an alternate embodiment, the first kernel layer, 405, is concentric with the second kernel layer, 406, but a portion of the first kernel layer contacts the first skin layer, 409, at the implant end. The first skin layer may be continuous around the entire implant, or it may be composed of a second material in the form of an end cap, 409, that contacts the first kernel layer. In a further embodiment, the first kernel layer, 410, is separated from the second kernel layer, 412, by a barrier layer, 411, that does not contain a therapeutic agent. Optional first, 413, and second, 414, skin layers may be present adjacent to the second kernel layer.

In certain embodiments, the first, second and third layers of the kernel are made from the same polymer. However, it can be envisioned that different polymers can be used for the first, second and third layers of the kernel so long as the first therapeutic agent in the kernel experiences a reduced permeation resistance as it is being released through the skin and meets the necessary release criteria needed to achieve a desired therapeutic effect.

In yet another series of embodiments, one or more skins can be medicated with one or more APIs. In certain embodiments, the first therapeutic agent is in dissolved form in the kernel and the second therapeutic agent is in solid form in the skin. As used herein, “solid” can include crystalline or amorphous forms. In certain embodiments, the first therapeutic agent is in solid form in the kernel and the second therapeutic agent is in solid form in the skin. In certain embodiments, the first therapeutic agent is in solid form in the kernel and the second therapeutic agent is in dissolved form in the skin. In certain embodiments, the first therapeutic agent is in the kernel of a reservoir-type system and the second therapeutic agent is in solid form in the skin. As used herein, solid can include crystalline or amorphous forms. In certain embodiments, the first therapeutic agent is in the kernel of a reservoir-type system and the second therapeutic agent is in dissolved form in the skin.

In one embodiment, the skin is non-resorbable. It may be formed of a medical grade silicone, as known in the art. Other examples of suitable non-resorbable materials include synthetic polymers selected from poly(ethers), poly(acrylates), poly(methacrylates), poly(vinyl pyrolidones), poly(vinyl acetates), including, but not limited to poly(ethylene-co-vinyl acetate), or ethylene vinyl acetate (EVA), poly(urethanes), celluloses, cellulose acetates, poly(siloxanes), poly(ethylene), poly(tetrafluoroethylene) and other fluorinated polymers, poly(siloxanes), copolymers thereof, and combinations thereof. The implant skin may also consist of a biocompatible metal such as titanium, nickel-titanium alloys, stainless steel, and others known in the art. In order to facilitate and control API release from the kernel, the metal skin may comprise a porous metal material as described above for kernel applications.

In one embodiment, one or more skins consist of the non-resorbable polymer expanded poly(tetrafluoroethylene) (ePTFE), also known in the art as Gore-Tex (99).

In another embodiment, the implant shell is resorbable. In one embodiment of a resorbable device, the sheath is formed of a biodegradable or bioerodible polymer. Examples of suitable resorbable materials include synthetic polymers selected from poly(amides), poly(esters), poly(ester amides), poly(anhydrides), poly(orthoesters), polyphosphazenes, pseudo poly(amino acids), poly(glycerol-sebacate), copolymers thereof, and mixtures thereof. In a preferred embodiment, the resorbable synthetic polymers are selected from poly(lactic acids), poly(glycolic acids), poly(lactic-co-glycolic acids), poly(caprolactones) (PCLs), and mixtures thereof. Other curable bioresorbable elastomers include PCL derivatives, amino alcohol-based poly(ester amides) (PEA) and poly(octane-diol citrate) (POC). PCL-based polymers may require additional cross-linking agents such as lysine diisocyanate or 2,2-bis(-caprolacton-4-yl)propane to obtain elastomeric properties.

In one embodiment, skins that are used to regulate or control the rate of API release from the kernel as well as the release kinetics (e.g., zero order versus first or second order) are microfabricated using methods known in the art and described herein, such as additive manufacturing. In some embodiments, the skin comprises a poly(caprolactones)/poly(lactic-co-glycolic acids) scaffold blended with tri-calcium phosphate constructed using solid freeform fabrication (SFF) technology (100), incorporated by reference in its entirety. In another embodiment, the skin comprises or consists of nanostructured elastomer thin films formed by casting and etching of a sacrificial templating agent (e.g., zinc oxide nanowires) such as described in the art (101), incorporated by reference in its entirety. In another embodiment, the skin comprises or comprises one or more elastomer thin films produced via highly reproducible, controllable, and scalable microfabrication methods; see, e.g., (102), incorporated by reference in its entirety. These include microelectromechanical systems (MEMS), nanoelectromechanical systems (NEMS) as well as microfluidic and nanofluidic systems known in the art. One embodiment, known in the art as soft lithography, involves the fabrication of a master with patterned features that may be reproduced in an elastomeric material by replica molding. Briefly, a substrate (typically a silicon wafer) is coated with photoresist (a photo-active polymer commonly used in photolithography, e.g., SU-8) and is exposed to UV radiation through a photomask to generate a desired pattern in the photoresist. The resist then is developed and the substrate etched so that the desired pattern is reproduced on the substrate in negative (i.e. channels and depressions in areas exposed to UV and not protected by photoresist). Skins are fabricated by replica molding, using the patterned master. Elastomer resin is poured onto a SU-8 patterned silicon master, and curing of the material against the master yields the desired pattern. Suitable elastomers include, but are not limited to poly-dimethyl siloxane (PDMS, silicone), thermoset polyester (TPE), photo-curable perfluoropolyethers (PFPEs). In another embodiment, patterned skins are fabricated using an embossing technique. A patterned master (stamp) is produced by methods known in the art, including soft lithography (vida supra), micromachining, laser machining, electrode discharge machining (EDM), electroplating, or electroforming. An elastomer in the form of a thin sheet is pressed against the master in a hydraulic press with applied heat to replicate the master pattern in the elastomer. Suitable elastomers for embossing include, but are not limited to, polylactic acid (PLA), polylactic-co-glycolic acid (PLGA), ethylene-co-vinylacetate (EVA), high-consistency rubber (HCR) silicone, polymethylmethacrylate (PMMA), polycarbonate (PC), cyclic olefin copolymer (COC), polystyrene (PS), polyvinylchloride (PVC), and polyethyleneterephthalate glycol (PETG).

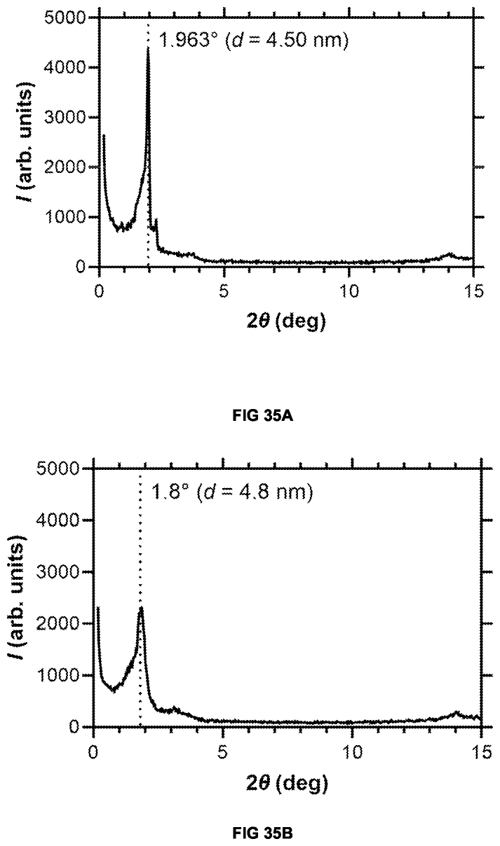

FIGS. 35A and 35B show illustrative drawings of skins made by microlithography. The grid-like pattern—analogous to that of an egg carton or waffle—comprises an array of dimples of well-defined shape (e.g., circle, square, hexagon, etc.), size (e.g., height and width), and draft (i.e., non-parallel walls) protruding from a thin film of defined thickness. Varying the density and physical characteristics of the surface features along with the film characteristics and composition can be used to control the API release kinetics (order and rate) from the kernel over a wide range.

Provided herein are devices comprising one skin or a plurality of skins. In some cases, the device comprises one skin. In some cases, the device comprises a plurality of skins.

In some cases, the skin covers part of the device or the entire device. In some cases, the skin covers part of the device. In some cases, the skin covers the entire device. In some cases, the skin comprises a rate-limiting skin.

In some cases, the skin is non-resorbable. In some cases, the skin comprises a biocompatible elastomer. In some cases, the skin comprises poly(dimethyl siloxane), silicone, one or more synthetic polymers, and/or metal. In some cases, the synthetic polymer is a poly(ether), poly(acrylate), poly(methacrylate), poly(vinyl pyrolidone), poly(vinyl acetate), poly(urethane), cellulose, cellulose acetate, poly(siloxane), poly(ethylene), poly(tetrafluoroethylene) and other fluorinated polymers, poly(siloxanes), copolymers thereof, or combinations thereof. In some cases, the polymer is expanded poly(tetrafluoroethylene) (ePTFE) or ethylene vinyl acetate (EVA). In some cases, the polymer is expanded poly(tetrafluoroethylene) (ePTFE). In some cases, the polymer is ethylene vinyl acetate (EVA).

In some cases, the metal is titanium, nickel-titanium (Nitinol) alloy, or stainless steel. In some cases, the metal is titanium or stainless steel. In some cases, the metal is titanium. In some cases, the metal is stainless steel.

In some cases, the skin is resorbable. In some cases, the skin comprises a biocompatible elastomer. In some cases, the skin comprises poly(amides), poly(esters), poly(ester amides), poly(anhydrides), poly(orthoesters), polyphosphazenes, pseudo poly(amino acids), poly(glycerol-sebacate), poly(lactic acids), poly(glycolic acids), poly(lactic-co-glycolic acids), poly(caprolactones) (PCLs), PCL derivatives, amino alcohol-based poly(ester amides) (PEA), poly(octane-diol citrate) (POC), copolymers thereof, or mixtures thereof. In some cases, the polymer is crosslinked PCL. In some cases, the crosslinked PCL comprises lysine diisocyanate or 2,2-bis(-caprolacton-4-yl)propane. In some cases, the polymer comprises poly(caprolactone)/poly(lactic-co-glycolic acid) and tri-calcium phosphate.

In some cases, the skin is fabricated via casting and etching, soft lithography, or microlithography. In some cases, the skin is fabricated via casting and etching. In some cases, the skin is fabricated via soft lithography. In some cases, the skin is fabricated via microlithography.

In some cases, the skin comprises a defined surface morphology. In some cases, the defined surface morphology comprises a grid pattern.

In some cases, the defined pores are microscopic or nanoscopic pores. In some cases, the defined pores are microscopic pores. In some cases, the defined pores are nanoscopic pores.

In some cases, the defined pores have a diameter of less than 2 nm. In some cases, the defined pores have a diameter of 0.1 nm, 0.5 nm, 1 nm, 1.5 nm, or 2 nm. In some cases, the defined pores have a diameter of 2 nm to 50 nm. In some cases, the defined pores have a diameter of 2 nm, 5 nm, 10 nm, 15 nm, 20 nm, 25 nm, 30 nm, 35 nm, 40 nm, 45 nm, or 50 nm. In some cases, the defined pores have a diameter greater than 50 nm.

### Resorbable and Biodegradable Devices

There are applications of the disclosure that benefit from resorbable and biodegradable devices. For the purposes of the current disclosure, “resorbable” is intended to mean a device that breaks down and becomes assimilated in vivo (e.g., resorbable sutures) while “biodegradable” is intended to mean a device that is capable of being decomposed by bacteria or other living organisms post use. Non-limiting, exemplary embodiments of both device types are given below.

### Resorbable Devices

The main advantage of resorbable devices is that, in certain cases, they do not need to be removed once their API cargo has been delivered. The resorbable implants described herein consist, at least in part, of materials that become degraded in vivo during the period of use. In some embodiments, the entire device is resorbable over the period of use. In some embodiments, in vivo degradation of the device occurs primarily after most of all the API cargo has been released. In certain embodiments, one or more of the device components (e.g., skin and/or kernel) described above comprises or comprises a resorbable elastomer (see “The Implant Skin” and “Implant Materials” for exemplary elastomers).

### Biodegradable Devices

The impetus for biodegradable implants predominantly arises from the desire to minimize the detrimental environmental impact post use, i.e., waste minimization. For example, IVRs delivering the antiretroviral drug dapivirine are currently being evaluated for HIV prevention in large-scale clinical trials (103, 104). These 28-day devices are made almost exclusively of silicone, which could result in a considerable waste burden if millions of women in sub-Saharan Africa regularly use the product, once approved. Over one million women around the world use the contraceptive IVR, NuvaRing®, which is predominantly made of EVA, another non-biodegradable elastomer creating further disposal concerns.

Compared to bioresorbable implants designed to degrade in the body to avoid the need for removal at the end of the period of use, biodegradable implants are designed to maintain integrity while inserted in the body, and to begin the degradation process once removed (i.e., post-use). One approach, similar to that used in biodegradable, disposable plastic items such as shopping bags and food containers, uses poly(lactic acid) polymers that are degraded by carboxyesterase enzymes produced by bacteria. An alternative approach is to utilize polymers that degrade in the presence of ultraviolet (UV) irradiation (i.e., sunlight). An important consideration is that the degradation process (and kinetics of degradation) be separated temporally from the period of use so that the delivery of the API is not impacted by the degradation process during the implant period of use.

### Other Design Considerations

### Considerations for the Delivery of Biomolecules

Due to their large molecular weight, hydrophilicity, and chemical/physical instability, biomolecules (e.g., peptides, peptide-drug conjugates, proteins, (ribo)nucleic acid oligomers) can be challenging to deliver in a controlled fashion from long-acting drug delivery devices. Many embodiments of the current disclosure overcome these limiting obstacles by immobilizing the biomolecules in porous kernels or water-soluble scaffolds (e.g., PVA nanofibers) encased in rate-limiting skins, such as ePTFE.

In addition to biomolecules that are approved by regulatory agencies to prevent or treat disease, the disclosure also serves as a platform to deliver exploratory agents for new applications. For example, in one non-limiting embodiment, messenger ribonucleic acids (mRNA's)—synthetic or natural—are delivered to stimulate the in vivo expression of one or more proteins (105), such as antibodies (106), and vaccine adjuvants (107). This approach has the advantage of leveraging the host's biochemical capabilities by stimulating it to synthesize the target agent in vivo, rather than delivering it directly from the implant. This can overcome high manufacturing costs of some biomolecules and their instability (e.g., cold-chain avoidance).

In some embodiments, certain excipients can improve the control of the biomolecule release rate from the implant (see “API Formulation”). For example, silk fibroin can be used to modulate the release rate of proteins, such as described by Zhang el al. (108), included herein by reference in its entirety.

In other embodiments, certain excipients can stabilize the biomolecules with respect to degradation or loss of biological activity using approaches known to those skilled in the art (109). Certain excipients stabilize biomolecules by creating a “water-like” environment in the dry state through hydrogen bonding interactions—e.g., sugars (110) and amino acids (111)—Other excipients create a glassy matrix that provides hydrogen bonding and immobilized the biomolecules to prevent aggregation that leads to loss of biologic activity (e.g., trehalose, inulin). Still other excipients can stabilize the pH in the implant formulation (e.g., buffer salts). Finally, surfactants can reduce the concentration of the biomolecules at the air-water interface during drying processes of formulation, decreasing shear stress and insoluble aggregate formation, and allowing the previously described stabilization mechanisms to occur throughout the drying process.

### In Vivo Localization of the Implant

In various embodiments, one or more radio-opaque materials (e.g., barium sulfate) are incorporated into the elastomer implant shell (i.e., API-impermeable polymer), or by making it into an end plug to be used to seal the shell (24, 112), incorporated herein by reference. The radio-opaque material can be integrated in the form of one or more band, or other shape, or dispersed throughout API-impermeable polymer. In various embodiments, the elastomer material making up part of the implant is coated with a metal (e.g., titanium) to make it radio-opaque, using any suitable process, such as those known in the art.

In certain embodiments, ultrasound is used to locate the implant. In these embodiments, polymers or polymer-additives (e.g., calcium) known in the art to be opaque to ultrasonography are employed to assist in visualizing the device in vivo.

The device may include at least one magnetic element to facilitate removal of the device (e.g., after API delivery has been completed) (113), incorporated herein by reference. In certain embodiments, the magnetic element may be located at the first end, the second end, or both the first and second ends of the cylindrical device. A soft polymeric coating may be provided over the magnetic elements.

To aid in implant insertion and/or removal, a hole may be punched, molded, or otherwise formed in one end of the implant. The hole may be used to grip the implant with forceps or another suitable tool. A loop made from suture material, wire, or other suitable material may be tied or otherwise attached to the hole to aid in gripping the implant for insertion and/or removal.

### Foreign Body Response

Silicone implants, for example, are inexpensive and wieldy, but may elicit a foreign-body reaction and are prone to migration. ePTFE implants are more biocompatible and capable of ingrowth, but expensive. Silicone-ePTFE composites have a silicone core and ePTFE liner and are used in surgical applications, such as rhinoplasty (114, 115) and cheek-lip groove rejuvenation (116), incorporated herein by reference. In one embodiment, the elastomer implant sheath is bonded to an outer ePTFE sleeve to form a composite (i.e., the ePTFE sleeve only serves to mitigate the foreign body response and does not control or affect API release from the device). In other embodiments, the ePTFE skin does play a role in controlling the API release rate from the device.

It is known in the art that the host's foreign body response can affect the safety of an implanted device, particularly for subcutaneous implants (117), or other types of devices implanted into body compartment. This reaction comprises protein adsorption on the implant surface, inflammatory cell infiltration, macrophage fusion into foreign body giant cells, fibroblast activation and ultimately fibrous encapsulation. This series of events may affect the function of subcutaneous implants, such as inhibition of API diffusion from long-acting API delivery depots and medical device failure. To date, combination approaches, such as hydrophilic coatings that reduce protein adsorption combined with delivery of dexamethasone are the most effective.

In a particular embodiment, the implantable drug delivery device releases one or more agents to mitigate or reduce the foreign body response in addition to the primary API. These agents are mixed with the API and any excipients, and formulated into the API kernel (see “API Formulation”, below). The agents are released from the implant with the API. In one embodiment, the agent included to reduce the foreign body response is a steroid. In one embodiment, this steroid is dexamethasone, or a dexamethasone derivative such as dexamethasone 21-acetate or dexamethasone 21-phosphate disodium salt.

Hydrogels, particularly zwitterionic hydrogels, can significantly reduce the foreign body response to subdermal implants. For further discussion, see, e.g., (118), incorporated by reference in its entirety.

### Implant Materials

In one embodiment, the implant drug delivery devices disclosed herein comprise one or more suitable thermoplastic polymers, elastomer materials, or metals suitable for pharmaceutical use. Examples of such materials are known in the art, and described in the literature (119, 120), incorporated by reference in their entirety.

In one embodiment, the implant elastomeric material is non-resorbable. It may comprise medical-grade poly(dimethyl siloxanes) or silicones, as known in the art. Exemplary silicones include without limitation fluorosilicones, i.e., polymers with a siloxane backbone and fluorocarbon pendant groups, such as poly(3,3,3-trifluoropropyl methylsiloxane. Other examples of suitable non-resorbable materials include: synthetic polymers selected from poly(ethers); poly(acrylates); poly(methacrylates); poly(vinyl pyrolidones); poly(vinyl acetates), including but not limited to EVA, poly(urethanes); celluloses; cellulose acetates; poly(siloxanes); poly(ethylene); poly(tetrafluoroethylene) and other fluorinated polymers, including ePTFE; poly(siloxanes); copolymers thereof and combinations thereof. The implant may also comprise or consist of a biocompatible metal such as titanium, nickel-titanium alloys (NiTi or Nitinol), stainless steel, and/or others known in the art.

In another embodiment, the implant elastomeric material is resorbable. In one embodiment of a resorbable device, the skin is formed of a biodegradable or bioerodible polymer. Examples of suitable resorbable materials include: synthetic polymers selected from poly(amides); poly(esters); poly(ester amides); poly(anhydrides); poly(orthoesters); polyphosphazenes; pseudo poly(amino acids); poly(glycerol-sebacate); copolymers thereof, and mixtures thereof. In one embodiment, the resorbable synthetic polymers are selected from poly(lactic acids), poly(glycolic acids), poly(lactic-co-glycolic acids), PCLs, and mixtures thereof. Other curable bioresorbable elastomers include PCL derivatives, amino alcohol-based PEAs and POC. PCL-based polymers may require additional cross-linking agents such as lysine diisocyanate or 2,2-bis(-caprolacton-4-yl)propane to obtain elastomeric properties.

In one embodiment of the implant drug delivery devices described herein, the elastomeric material comprises suitable thermoplastic polymer or elastomer material that can, in principle, be any thermoplastic polymer or elastomer material suitable for pharmaceutical use, such as silicone, low density polyethylene, EVA, polyurethanes, and styrene-butadiene-styrene copolymers.

In certain embodiments, EVA is used in the kernel and the skin due to its excellent mechanical and physical properties. The EVA material may be used for the kernel, as well as the skin and can be any commercially available EVA, such as the products available under the trade names: Elvax, Evatane, Lupolen, Movriton, Ultrathene and Vestypar.

The permeability of EVA copolymers for small to medium sized API molecules (Ms 600 g mol⁻¹) is primarily determined by the vinyl acetate to ethylene ratio. Low-VA content EVA copolymers are substantially less permeable than high VA-content skins and hence display rate limiting properties if used as skin. EVA copolymers with VA-content of 19% w/w or less (519% w/w) are substantially less permeable than polymer having VA-content above and including 25% w/w (>25% w/w).

In some embodiments, the first thermoplastic polymer is an EVA and has a vinyl acetate content of 28% or greater. In other embodiments, the first thermoplastic polymer has a vinyl acetate content of greater than 28%. In still other embodiments, the first thermoplastic polymer has a vinyl acetate content between 28-40% vinyl acetate. In yet other embodiments, the first thermoplastic polymer has a vinyl acetate content between 28-33% vinyl acetate. In one embodiment, the first thermoplastic polymer has a vinyl acetate content of 28%. In one embodiment, the first thermoplastic polymer has a vinyl acetate content of 33%. In some embodiments, the second thermoplastic polymer is an ethylene-vinyl acetate copolymer and has a vinyl acetate content of 28% or greater. In other embodiments, the second thermoplastic polymer has a vinyl acetate content of greater than 28%. In still other embodiments, the second thermoplastic polymer has a vinyl acetate content between 28-40% vinyl acetate. In yet other embodiments, the second thermoplastic polymer has a vinyl acetate content between 28-33% vinyl acetate. In one embodiment, the second thermoplastic polymer has a vinyl acetate content of 28%. In one embodiment, the second thermoplastic polymer has a vinyl acetate content of 33%.

In some embodiments, the second thermoplastic polymer is an EVA and has a vinyl acetate content of 28% or less. In other embodiments, the second thermoplastic polymer has a vinyl acetate content of less than 28%. In still other embodiments, the second thermoplastic polymer has a vinyl acetate content between 9-28% vinyl acetate. In yet other embodiments, the second thermoplastic polymer has a vinyl acetate content between 9-18% vinyl acetate. In one embodiment, the second thermoplastic polymer has a vinyl acetate content of 15%. In one embodiment, the second thermoplastic polymer has a vinyl acetate content of 18%.

It should be noted that when a specific vinyl acetate content, e.g., 15%, is mentioned, it refers to the manufacture's target content, and the actual vinyl acetate content may vary from the target content by plus or minus 1% or 2%. One of ordinary skill in the art would appreciate that suppliers may use internal analytical methods for determining vinyl acetate content, thus there may be an offset between methods.

### Other Long-Acting API Delivery Platforms

The delivery of peptide-drug conjugates, in certain cases in combination with other APIs, from other long-acting systems can be beneficial for preventing, treating, and/or managing a disease of medical disorder. In one non-limiting embodiment, the formulation is injected into a body cavity (e.g., subcutaneous, intramuscular, intratumoral) as a particle suspension. As used herein, solid particles can include crystalline or amorphous forms. In one non-limiting embodiment, the particles consist of pure API, or API mixed with one or more excipients. In another non-limiting embodiment, the particles consist of the API suspended in a polymer matrix, by analogy to the various systems described for implants above. In another non-limiting example, the above particles are coated with one or more polymer skins. In another non-limiting embodiment, the API is injected as a solution or suspension in a system that hardens in vivo to form an implant in situ (e.g., Atrigel system), as described above.

In one embodiment, the size distribution of the solid particles is polydisperse. In one embodiment, the size distribution of the solid particles is monodisperse. In one embodiment, the solid particles comprise or consist of nanoparticles (mean diameter<100 nm). In one embodiment, the mean diameter of the particles can range from 1-10 nm, 10-25 nm, 25-100 nm, and 100-500 nm. Suitable mean microparticle diameters can range from 0.5-50 μm, from 0.5-5 μm, from 5-50 μm, from 1-10 μm, from 10-20 μm, from 20-30 μm, from 30-40 μm and from 40-50 μm. Other suitable mean particle diameters can range from 50-500 μm, from 50-100 μm, from 100-200 μm, from 200-300 μm, from 300-400 μm, from 400-500 μm, and from 0.5-5 mm. Suitable particle shapes include spheres, needles, rhomboids, cubes, and irregular shapes.

In another non-limiting embodiment, the API is delivered from a microarray (microneedle) patch. This delivery platform is well-known in the art, as described in (121, 122), included herein by reference in their entirety. The microarray patch can be formulated with one or more APIs in solution or suspension using resorbable or non-resorbable polymers. In one embodiment, one or more APIs are delivered intradermally via microarray patch. In another non-limiting embodiment, a microarray patch is used to achieve ocular delivery of one or more APIs as described in (123, 124), included herein by reference in their entirety.

### Formulation Considerations

The API formulation can include essentially any therapeutic, prophylactic, or diagnostic agent that would be useful to deliver locally to a body cavity.

### Target In Vivo API Release Kinetics and Profiles

The API formulation may provide a temporally modulated release profile or a more continuous or consistent release profile. Pulsatile release can be achieved from a plurality of kernels, implanted simultaneously or in a staggered fashion over time. For example, different degradable skins can be used to by temporally stagger the release of one or more agents from each of several kernels.

### Choice of Drug

The API formulation can include essentially any therapeutic, prophylactic, or diagnostic agent that would be useful for delivery to an anatomic compartment. The drug delivery devices disclosed herein comprise at least one pharmaceutically active substance, including, but not limited to, agents that are used in the art for the applications described under “Use and Applications of the Device”, and combinations thereof. In one embodiment, the drug delivery device comprises two or more pharmaceutically active substances. In this instance, the pharmaceutically active substances can have the same hydrophilicity or hydrophobicity or different hydrophilicities or hydrophobicities. At least one of the APIs delivered from a long-acting device consists of a peptide-drug conjugate. Non-limiting examples of drugs are provided below.

Non-limiting examples of hydrophobic pharmaceutically active substances include: cabotegravir, dapivirine, fluticasone propionate, chlordiazepoxide, haloperidol, indomethacin, prednisone, and ethinyl estradiol. Non-limiting examples of hydrophilic pharmaceutically active substances include: acyclovir, tenofovir, atenolol, aminoglycosides, exenatide acetate, leuprolide acetate, acetylsalicylic acid (aspirin), and levodopa.

In some cases, the pharmaceutically active substance is chloroquine or hydroxychloroquine, pharmaceutically acceptable salts thereof, or combinations thereof. In some cases, the pharmaceutically acceptable salt is a phosphate, such as a diphosphate, or a chloride, such as a dichloride, or combinations thereof.

In some cases, the pharmaceutically active substance is an antibacterial agent. In some cases, the antibacterial agent is a broad-spectrum antibacterial agent. Non-limiting examples of antibacterial agents include azithromycin.

In some cases, the pharmaceutically active substance is an antiviral agent. Non-limiting examples of antiviral agents include remdesivir (Gilead Sciences), acyclovir, ganciclovir, and ribavirin, and combinations thereof. In some cases, the pharmaceutically active substance is an antiretroviral drug. In some cases, the antiretroviral drug is used to treat HIV/AIDS. Non-limiting examples of antiretroviral drugs include protease inhibitors. In some cases, the antiviral agent is one or more antiretroviral drugs used for pre-exposure prophylaxis (PrEP). In some cases, the antiretroviral drug is GSK2838232 (8232), islatravir (ISL), rilpivarine (RPV), cabotegravir (CAB), emtricitabine (FTC), tenofovir (TFV), dolutegravir (DTG), lamivudine (3TC), or a combination thereof. In some cases, the antiviral agent is a prodrug of an antiretroviral drug. In some cases, the antiviral agent is a prodrug of emtricitabine, tenofovir, dolutegravir (DTG), lamivudine (3TC), or a combination thereof. In some cases, the antiviral agent is cabotegravir, tenofovir disoproxil fumarate (TDF), tenofovir diphosphate (TFV-DP), tenofovir alafenamide (TAF), emtricitabine, or a combination thereof. In some cases, the antiviral agent is dolutegravir (DTG), lamivudine (3TC), or a combination thereof.

In some cases, the pharmaceutically active substance is an agent that affects immune and fibrotic processes. Non-limiting examples of agents that affect immune and fibrotic processes include inhibitors of Rho-associated coiled-coil kinase 2 (ROCK2), for example, KD025 (Kadmon).

In some cases, the pharmaceutically active substance is a sirtuin (SIRT1-7) inhibitor. In some cases, the sirtuin inhibitor is EV-100, EV-200, EV-300, or EV-400 (Evrys Bio). In some cases, administration of a sirtuin inhibitor restores a human host's cellular metabolism and immunity.

The pharmaceutically active substances described herein can be administered alone or in combination. Combinations of pharmaceutically active substances can be administered using one implant or multiple implants. In some cases, the drug delivery devices described here comprise one pharmaceutically active substance. In some cases, the drug delivery devices described herein comprise more than one pharmaceutically active substance. In some cases, the drug delivery devices described herein comprise a combination of pharmaceutically active substances. In some cases, the combination of pharmaceutically active substances is chloroquine and azithromycin, hydroxychloroquine and azithromycin, lopinavir and ritonavir, KD025 and ribavirin, KD025 and remdesivir, EV-100 and ribavirin, or EV-100 and remdesivir.

In one embodiment, HIV and HBV can be treated and/or prevented using one or more implants delivering potent antiviral agents, including but not limited to combinations of tenofovir alafenamide, potent prodrugs of lamivudine (3TC), and dolutegravir (DTG).

In one embodiment, an IVR delivering two or more APIs against HIV can be advantageous. Non-limiting examples include tenofovir disoproxil fumarate (TDF) and emtricitabine (FTC) in combination with a third anti-HIV compound from a different mechanistic class such as DTG, elvitegravir, the antiviral peptide C5A, and broadly neutralizing antibodies against HIV, such as VRC01. In some embodiments, TDF is used without FTC in these combinations. In other embodiments, FTC is used without TDF in these combinations.

The suitability of any given pharmaceutically active substance is not limited or predicated by any given medical application, but rather is a function of the following non-limiting parameters:

Potency; the potency of the API will determine whether it can be formulated into a long-acting delivery system (e.g., one or more drug delivery devices) and maintain pharmacologically relevant concentrations in the key anatomic compartment(s) for the target duration of use (see “Example 1”). In some cases, it may only be possible to use one implant at a time, depending on the anatomic compartment (e.g., IVR).

Implant Payload; the amount of API that can be formulated into long-acting delivery systems, and the number of feasible devices implanted, or the size of the injection (volume of injection of area of patch), at one time, together with the API potency is a primary limiting factor in selecting an API for a given application (see “Example 1” and “Example 2”).

Solubility; the aqueous solubility of the API must be such that delivery via implant is achievable at the target rate. The solubility, and hence release rate, of the API also can be modulated (increased or decreased) using suitable excipients, by preparing pharmaceutically acceptable salts, and via conjugation into prodrugs all well-known in the art, as well as formulation strategies as described above.

Targeted Delivery; implant API delivery, as disclosed herein, can target the systemic circulatory system (e.g., subcutaneous or intramuscular implants) or local compartments (e.g., vaginal or ocular devices).

Local Toxicity; the systemic toxicity profile of many APIs envisioned in the disclosed application will have been determined prior to formulation into implants, especially when FDA-approved agents are used. Local toxicity at the implantation site therefore represent the largest safety concern in these cases, and could limit the API delivery rate. In some cases, drugs have a low therapeutic index (TI) and it may not be possible to control the drug release rate from the implant to provide safe and effective concentrations in the target pharmacologic compartment.

Cost; the API cost and/or the manufacturing cost could be limiting in certain cases.

In silico prediction of implant specifications for any given API and medical application and the development of a Target Product Profile is highly challenging, as known in the art for other sustained release drug delivery technologies, and usually requires preclinical studies followed by clinical validation of the pharmacology in terms of pharmacokinetics (PK) and pharmacodynamics (PD, safety and efficacy).

### API Formulation

The API formulation may consist only of the API, or may include one or more other agents and/or one or more pharmaceutically acceptable excipients. Pharmaceutically acceptable excipients are known in the art and may include: viscosity modifiers, bulking agents, surface active agents, dispersants, disintegrants, osmotic agents, diluents, binders, anti-adherents, lubricants, glidants, pH modifiers, antioxidants and preservants, and other non-active ingredients of the formulation intended to facilitate handling and/or affect the release kinetics of the API.

In some embodiments, the binders and/or disintegrants may include, but are in no way limited to, starches, gelatins, carboxymethylcellulose, croscarmellose sodium, methyl cellulose, ethyl cellulose, hydroxymethyl cellulose, hydroxyethyl cellulose, hydroxypropyl cellulose, hydroxypropylmethyl cellulose, hydroxypropylethyl cellulose, hydroxypropylmethyl cellulose, microcrystalline cellulose, polyvinylpyrrolidone, polyethylene glycol, sodium starch glycolate, lactose, sucrose, glucose, glycogen, propylene glycol, glycerol, sorbitol, polysorbates, and colloidal silicon dioxide. In certain embodiments, the anti-adherents or lubricants may include, but are in no way limited to, magnesium stearate, stearic acid, sodium stearyl fumarate, and sodium behenate. In some embodiments, the glidants may include, but are in no way limited to, fumed silica, talc, and magnesium carbonate. In some embodiments, the pH modifiers may include, but are in no way limited to, citric acid, lactic acid, and gluconic acid. In some embodiments, the antioxidants and preservants may include, but are in no way limited to ascorbic acid, butylated hydroxytoluene (BHT), butylated hydroxyanisole (BHA), cysteine, methionine, vitamin A, vitamin E, sodium benzoate, and parabens.

### Effect of Excipients on API Release

The devices disclosed herein can comprise excipients to facilitate and/or control the release of the API from the devices. Non-limiting examples of these excipients include PEG and TEC. It is contemplated that release kinetics of APIs can be modulated by the incorporation of different excipients into the devices disclosed herein. That is, the release kinetics of the API can be tuned over a wide range by changing the nature and/or amount of the excipient contained therein. In some cases, the devices contain low concentrations of excipient, e.g., from about 0% to about 30% excipient by weight. In some cases, the excipient is a polyether or an ester. In some cases, the excipient is PEG or TEC. In some cases, the devices comprise PEG to achieve a lower, sustained release of an API. In some cases, the devices comprise TEC to achieve a more immediate, larger dose of an API.

### Target Implant Specifications

The amount of pharmaceutically active substance(s) incorporated into the implant device can also be calculated as a pharmaceutically effective amount, where the devices of the present implants comprise a pharmaceutically effective amount of one or more pharmaceutically active substances. By “pharmaceutically effective”, it is meant an amount that is sufficient to effect the desired physiological or pharmacological change in subject. This amount will vary depending upon such factors as the potency of the particular pharmaceutically active substance, the density of the pharmaceutically active substance, the shape of the implant, the desired physiological or pharmacological effect, and the time span of the intended treatment.

In some embodiments, the pharmaceutically active substance is present in an amount ranging from about 1 mg to about 25,000 mg of pharmaceutically active substance per implant device. This includes embodiments in which the amount ranges from about 2 mg to about 25 mg, from about 25 mg to about 250 mg, from about 250 mg to about 2,500 mg, and from about 2,500 to about 25,000 mg of pharmaceutically active substance per implant device.

The size of the API depot will determine the maximum amount of pharmaceutically active substance in the implant. For example, subdermal implants traditionally consist of cylinder-shaped devices 2-5 mm in diameter and 40 mm in length. The maximum amount of pharmaceutically active substance per implant device of this nature would be less than 1,000 mg. A typical IVR weighs less than 10 g, which means that the maximum amount of pharmaceutically active substance per implant device of this nature would be less than 10 g.

In certain embodiments of the implant drug delivery device described herein, wherein the first therapeutic agent is present in the kernel about 0.1%-99% w/w. In other embodiments, the first therapeutic agent is present in the kernel at about 0.1-1% w/w, at about 1-5% w/w, at about 5-25% w/w, at about 25-45% w/w, at about 45-65% w/w, at about 65-100% w/w, at about 65-75% w/w, or at about 75-85% w/w, or about 85-99% w/w.

In certain embodiments, the implant drug delivery devices described herein are capable of releasing the therapeutic agents contained therein over a period of 1, 2, 3, 4, 5, or 6 weeks. In certain embodiments, the implant drug delivery devices described herein are capable of releasing the therapeutic agents contained therein over a period of 8, 10, 12 or 14 weeks. In certain embodiments, the implant drug delivery devices described herein are capable of releasing the therapeutic agents contained therein over a period of 1, 2, 3, or 6 months. In certain embodiments, the implant drug delivery devices described herein are capable of releasing the therapeutic agents contained therein over a period of one, two, three, or four years.

In one embodiment, the subdermal implant drug delivery system device described herein, is capable of releasing a peptide conjugate of tenofovir alafenamide (TAF), or its pharmaceutically acceptable salts, over a period of 3, 4, 6, or six weeks or 8, 10, 12 or 14 weeks, or 1, 2, 3, 6, or 12 months at an average rate of between 0.05-3 mg d⁻¹. In certain embodiments, the subdermal implant described herein is capable of releasing a peptide conjugate of TAF, or its pharmaceutically acceptable salts, at an average rate of between 0.1-2 mg d⁻¹. In certain embodiments, the subdermal implant described herein is capable of releasing a peptide conjugate of TAF, or its pharmaceutically acceptable salts, over a period of 3, 6, or 12 months at a rate of between 0.1-1 mg d⁻¹. In certain embodiments, the subdermal implant described herein is capable of releasing a peptide conjugate of TAF, or its pharmaceutically acceptable salts, at an average rate of 0.25 mg d⁻¹. In certain embodiments, the subdermal implant described herein is capable of releasing a peptide conjugate of TAF, or its pharmaceutically acceptable salts, at an average rate of 0.5 mg d⁻¹. In certain embodiments, the subdermal implant described herein is capable of releasing a peptide conjugate of TAF, or its pharmaceutically acceptable salts, at an average of 1 mg d⁻¹.

In certain embodiments of the implant drug delivery devices described herein, a second therapeutic agent is present in the skin at about 5-50% w/w. In other embodiments, the second therapeutic agent is present in the skin at about 10-50% w/w, at about 20-50% w/w, at about 10%, 30% or 50% w/w of the skin.

In certain embodiments, the implant drug delivery devices described herein are stable at room temperature. As used herein, “room temperature” lies anywhere between about 18° C. and about 30° C. As used herein, a physically implant drug delivery device is a device which can be stored at about 18-30° C. for at least about one month.

### Implant Fabrication

Also described herein are methods of manufacturing the implant drug delivery devices.

Implant Fabrication Involving API and/or Excipient in Polymer Dispersions

Implants where the API and/or excipient is dissolved or suspended in solid form in the elastomer (e.g., matrix type implant devices) are fabricated using methods known in the art. For example, an extrusion process can be used. Elastomer pellets cryomilled to a powder are blended with API substance powder. Alternatively, API substances may be directly combined with elastomer pellets prior to introduction to the extruder, or mixing of API substance and elastomer pellets may be a continuous process that controls mass flow rate of API substance and elastomer to the extrusion screw to achieve a desired API:polymer ratio. API substance concentrations over a wide range, from 0.1-99% w/w, can be used with this approach. The API and polymer blends are hot-melt extruded to produce the implant drug product.

Also described herein are methods of manufacturing the implants where the API and/or excipient is dissolved or suspended in solid form in the elastomer (e.g., matrix type implant devices) described herein comprising:

Producing a homogenous polymer kernel granulate comprising the first therapeutic agent and a loaded skin layer granulate comprising the second therapeutic agent, or simply an unmedicated skin,

Co-extruding the kernel granulate comprising the first therapeutic agent and the skin layer granulate comprising the second therapeutic agent (or unmedicated) to form a two-layered drug delivery device or co-extruding the kernel granulate comprising the first therapeutic agent with additional kernel layers and/or the skin granulate comprising the second therapeutic agent (or unmedicated) with additional skin layers to form a multi-layered drug delivery device.

Also described herein are methods of manufacturing the drug loaded kernel or skin granulate:
a) Grinding the polymer,
b) Dry powder mixing the ground polymer with the respective active compound,
c) Blend-extruding the resulting powder mixtures of Step (b),
d) Cutting the resulting loaded polymer strands into granules, thereby obtaining a kernel granulate and/or the skin layer granulate,
e) When required lubricating the granulate prior to coextrusion.

### Reservoir Implant Fabrication

Also described herein are methods of manufacturing the implant drug delivery devices of the reservoir design.

In one embodiment of reservoir-type implants, the API, and any other solid agents or excipients, can be filled into the implant shell as a powder or slurry using filling methods known in the art. In another embodiment, the solid actives and carriers can be compressed into microtablet/tablet form to maximize the loading of the actives (24, 112), using means common in the art.

In one example, the drug formulation is in the form of a solid drug rod. Embodiments of drug rods, and methods of making such drug rods, are described in the art, such as (125), incorporated by reference in its entirety. The drug rods may be formed by adapting other extrusion or casting techniques known in the art. For example, a drug rod comprising an API may be formed by filling a tube with an aqueous solution of the API and then allowing the solution to evaporate. As another example, a drug rod comprising of an API may be formed by extrusion, as known in the art. In many embodiments, the drug formulation desirably includes no or a minimum quantity of excipient for the same reasons of volume/size minimization.

Open ends of the implant can be plugged with a pre-manufactured end plug to ensure a smooth end and a solid seal, 500. Plugs may be sealed in the implant end using frictional force (for example, a rim and groove that lock together to form a seal); an adhesive; induction or laser welding, or another form of heat sealing that melts together the plug and implant end. In another embodiment, the ends are sealed without using a solid plug by one of a number of methods known to one skilled in the art, including but not limited to, heat-sealing, induction welding, laser welding, or sealing with an adhesive, 501.

### Fabrication of Porous Implant Components

Porous material or materials can be used in implant fabrication either for the kernel or the skin, as described in detail above. In one embodiment, the API permeable portion of an implant device is formed from a porous membrane of polyurethane, silicone, or other suitable elastomeric material. Open cell foams and their production are known to those skilled in the art (126). Open cell foams may be produced using blowing agents, typically carbon dioxide or hydrogen gas, or a low-boiling liquid, present during the manufacturing process to form closed pores in the polymer, followed by a cell-opening step to break the seal between cells and form an interconnected porous structure through which diffusion may occur. An alternative embodiment employs a breath figure method to create an ordered porous polymer membrane for API release (127). In this method, a hexagonal array of micrometric pores is obtained by water droplet condensation during fast solvent evaporation performed under a humid flow. Porous membranes may also be fabricated using porogen leaching methods (128), whereby a polymer is mixed with salt or other soluble particles of controlled size prior to casting, spin-coating, extrusion, or other processing into a desired shape. The polymer composite is then immersed in an appropriate solvent, as known in the art, and the porogen particles are leached out leaving structure with porosity controlled by the number and size of leached porogen particles. A preferred approach is to use water-soluble particles and water as the solvent for porogen leaching and removal. Highly porous scaffolds with porosity values up to 93% and average pore diameters up to 500 μm can be formed using this technique. A variant of this method is melt molding and involves filling a mold with polymer powder and a porogen and heating the mold above the glass-transition temperature of the polymer to form a scaffold. Following removal from the mold, the porogen is leached out to form a porous structure with independent control of morphology (from porogen) and shape (from mold).

A phase separation process can also be used to form porous membranes (128). A second solvent is added to a polymer solution (quenching) and the mixture undergoes a phase separation to form a polymer-rich phase and a polymer-poor phase. The polymer-rich phase solidifies and the polymer poor phase is removed, leaving a highly porous polymer network, with the micro- and macro-structure controlled by parameters such as polymer concentration, temperature, and quenching rate. A similar approach is freeze drying, whereby a polymer solution is cooled to a frozen state, with solvent forming ice crystals and polymer aggregating in interstitial spaces. The solvent is removed by sublimation, resulting in an interconnected porous polymer structure (128). A final method for forming porous polymer membranes is using a stretching process to create an open-cell network (129).

Porous metal materials may be fabricated by traditional sintering processes (130, 131). Loose powder or gravity sintering creates pores from the voids in the packed powder as grains join by a diffusional bonding process. Pore size and density is determined primarily by the morphology of the starting metal powder material and is difficult to control. Porogens may be used to create open-cell, interconnected metal foams of ca. 35-80% porosity with 100-600 μm pore size in a method analogous to those described herein for polymer foams. Porogens may include salts (e.g., NaCl, NaF, and NH₄HCO₃), organic materials [e.g., tapioca starch (132), urea (133-135)], or other metals (e.g., magnesium). Porogens are removed to form pores thermally during sintering or in a post-sintering process, or by dissolution in a solvent. The high melting temperature (1310° C.) of Nitinol limits preparation methods of porous materials to powder metallurgy techniques (136). Materials can be prepared by sintering of Ni and Ti powders in predetermined ratios to form NiTi alloys during the sintering process. Alternatively, pre-alloyed NiTi powders may be sintered with or without additional porogens to form porous structures with controlled Ni:Ti ratios.

### Additive Manufacturing of Implant Components

Additive manufacturing—colloquially referred to as 3D printing technology in the art—is one of the fastest growing applications for the fabrication of plastics. Components that make up the implant can be fabricated by additive techniques that allow for complex, non-symmetrical three-dimensional structures to be obtained using 3D printing devices and methods, such as those known to those skilled in the art (137, 138), incorporated herein by reference. There are currently three principal methods for additive manufacturing: stereolithography (SLA), selective laser sintering (SLS), and fused deposition modeling (FDM).

The SLA process requires a liquid plastic resin, a photopolymer, which is then cured by an ultraviolet (UV) laser. The SLA machine requires an excess amount of photopolymer to complete the print, and a common g-code format may be used to translate a CAD model into assembly instructions for the printer. An SLA machine typically stores the excess photopolymer in a tank below the print bed, and as the print process continues, the bed is lowered into the tank, curing consecutive layers along the way. Due to the smaller cross-sectional area of the laser, SLA is considered one of the slower additive fabrication methods, as small parts may take hours or even days to complete. Additionally, the material costs are relatively higher, due to the proprietary nature and limited availability of the photopolymers. In one embodiment, one or more components of the implant is fabricated by an SLA process.

The SLS process is similar to SLA, forming parts layer by layer through use of a high energy pulsed laser. In SLS, however, the process starts with a tank full of bulk material in powder form. As the print continues, the bed lowers itself for each new layer, advantageously supporting overhangs of upper layers with the excess bulk powder not used in forming the lower layers. To facilitate processing, the bulk material is typically heated to just under its transition temperature to allow for faster particle fusion and print moves, such as described in the art (139). In one embodiment, one or more components of the implant is fabricated by an SLS process.

Porous metal materials formed by traditional sintering can suffer from inherent brittleness of the final product and limited control of pore shape and distribution. Additive manufacturing techniques can overcome some of these limitations and improve control of various pore parameters and mechanical properties, and allow fabrication of parts with complex shape and geometry. These include techniques that use a powder bed such as SLS (140), selective laser melting (SLM) (86, 141). Aluminum and titanium composites can be produced by SLS with control of porosity and mechanical properties by varying laser power: with low power (25-40 W), materials exhibit higher porosity and lower mechanical strength; at higher laser power (60-100 W), dense parts were formed with macroporosity generated from the implant structural design (142) Advanced manufacturing processes may be based on layered manufacturing to produce parts additively. CAD/CAM based layered manufacturing techniques have found applications in the near net shape fabrication of porous parts with controlled porosity. Electron Beam Melting (EBM) and Direct Metal Laser Sintering (DMLS) processes allow a direct digitally enabled fabrication of porous custom titanium implants with a controlled porosity and desired external and internal characteristics (143, 144). Typically, these rapid manufacturing technologies are utilized in aerospace applications but the systems can be easily extended for use in the fabrication of medical implants. EBM is a direct CAD to metal rapid prototyping process that can produce dense and porous metal parts by melting metal powder layer by layer with an electron beam, resulting in directed solidification of the metal powder into a predetermined 3D structure. The SLS and SLM processes are similar, but use a laser to melt the powder, typically producing a more-dense structure. Direct 3D deposition and sintering of Ti alloy fibers can produce scaffolds of controlled porosity 100-700 μm) and total porosity as high as 90% (145-147). An alternative is Laser Engineered Net Shape (LENS) processing, an additive manufacturing technology developed for fabricating metal parts directly from a computer-aided design (CAD) solid model by using a metal powder injected into a molten pool created by a focused, high-powered laser beam (140, 148).

Rather than using a laser to form polymers or sinter particles together, FDM works by extruding and laying down consecutive layers of materials at high temperature from polymer melts, allowing adjacent layers to cool and bond together before the next layer is deposited. In the most common FDM approach, fused fiber fabrication (FFF), polymer in the form of a filament is continuously fed into a heated print head print whereby it melts and is deposited onto the print surface. The print head moves in a horizontal plane to deposit polymer in a single layer, and either the print head or printing platform moves along the vertical axis to begin a new layer. A second FDM approach uses a print head design based on a traditional single-screw extruder to melt polymer granulate (powders, flakes, or pellets) and force the polymer melt through a nozzle whereby it is deposited on the print surface similar to FFF. This approach allows the use of standard polymer materials in their granulated form without the requirement of first fabricating filaments through a separate extrusion step. In one embodiment, one or more components of the implant is fabricated by an FDM and/or FFF process.

In another embodiment, Arburg Plastic Freeforming (APF) (149) is the additive manufacturing technique used in implant fabrication. In this embodiment, a plasticizing cylinder with a single screw is used to produce a homogeneous polymer melt similarly to the process for thermoplastic injection molding. The polymer melt is fed under pressure from the screw cylinder to a piezoelectrically actuated deposition nozzle. The nozzle discharges individual polymer droplets of controlled size in a pre-calculated position, building up each layer of the 3-dimensional polymer print from fused droplets. The screw and nozzle assembly is fixed in location, and the build platform holding the printed part is moved along three axes to control droplet deposition position. The droplets bond together on cooling to form a solid part. This technique can operate at elevated temperatures (ca. 300° C.) and pressures (ca. 400 bar). One advantage of the APF method is that it is directly compatible with many of the processes used in injection molding and extrusion (e.g., granulated polymer feedstocks, no organic solvents).

In another embodiment, droplet deposition modelling (DDM) is used as the additive manufacturing technique by producing discrete streams of material during deposition, well-known in the art for inkjet systems.

A preferred method of additive manufacturing that avoids sequential layer deposition to form the three-dimensional structure is to use continuous liquid interface production (CLIP), a technique recently developed by Carbon3D. In CLIP, three dimensional objects are built from a fast, continuous flow of liquid resin that is continuously polymerized to form a monolithic structure with the desired geometry using UV light under controlled oxygen conditions. The CLIP process is capable of producing solid parts that are drawn out of the resin at rates of hundreds of mm per hour. Implant scaffolds containing complex geometries may be formed using CLIP from a variety of materials including polyurethane and silicone.

In one embodiment, the implants are manufactured under fully aseptic conditions. In another embodiment, the implants are terminally sterilized using methods known in the art such as gamma sterilization, steam sterilization, dry heat sterilization, UV irradiation, ethylene oxide sterilization, and the like.

### Methods for Implantation & Removal of the Device

Methods for insertion and removal of IVRs, or other vaginal devices such as IUDs, pessaries, and the like are known in the art. Similar methods can be used for embodiments where the implantable device is a vaginal drug delivery device.

Implantation embodiments describing subdermal or intramuscular drug delivery devices are described herein. In some cases, the drug delivery device is a subdermal or intramuscular implant, an intravaginal ring, or a microarray patch. In some cases, the drug delivery device is a subdermal implant. In some cases, the drug delivery device is an intramuscular implant. In some cases, the drug delivery device is an intravaginal ring. In some cases, the drug delivery device is a microarray patch. In some embodiments, one or more devices are implanted together. In one embodiment, insertion and removal are carried out by a medical professional.

The devices of the present disclosure can be implanted into a subject/patient in accordance with standard procedures by trained professionals. The term “subject/patient” includes all mammals (e.g., humans, valuable domestic household, sport or farm animals, laboratory animals). In one embodiment, insertion could instead by facilitated using a trocar to ease access. Such device insertion—and removal (150, 151)—are described in the art for example subdermal implants, and are incorporated herein in full by reference (152, 153).

Dissolvable/resorbable implants are not anticipated to require removal under normal conditions.

Identification of targeted implant for removal is identified by palpation as well as use of imaging technique (ultrasound, magnetic detection, infrared, X-ray, or similar methods) based on the anticipated tracking markers incorporated into implant production.

Once identified, local anesthetic is applied topically/injected at the distal end of implant where small incision will be made (usually ca. 2-6 mm) enabling identifying the implant and/or retrieving adaptation (hole) using standard blunt-ended forceps or similar to visually identify the implant and grab/hook the distal end. Mosquito or similar forceps are then inserted to grab the end.

Based on implant components as well as individual responses, most are able to be grasped and pulled directly out without complications. Many are able to be “pushed out” with manual/instrument pressure on the back end/proximal implant end.

### Implant Sheaths

ePTFE has been used in the art as a sheath material to line the pocket where saline or silicone gel breast implants are surgically placed (vide supra). The ePTFE liners allow implants to integrate with the body by tissue in-growth without capsule (scar tissue) formation and also prevent the pocket from closing on itself, thus keeping the pocket open. A typical ePTFE liner is 0.35 mm thick and has a micro porosity of about 40 μm. This allows the body to grow into pores without forming a fibrous scar around the material. The material is permanent and does not degrade within the body. It can be removed if necessary. In some embodiments, an ePTFE liner is placed in a subdermal pocket where the implant(s) is(are) located, reducing the foreign body response and facilitating implant replacement for continuous therapies. In some embodiments, an ePTFE liner is placed in a intramuscular pocket where the implant(s) is(are) located, reducing the foreign body response and facilitating implant replacement for continuous therapies.

Provided herein are devices for implantation into the body of a patient. In some cases, implantation into the body comprises implantation into a sterile anatomic compartment. In some cases, the sterile anatomic compartment is selected from the subcutaneous space, the intramuscular space, the eye, the ear, and the brain. In some cases, the sterile anatomic compartment is the subcutaneous space. In some cases, the sterile anatomic compartment is the intramuscular space. In some cases, the sterile anatomic compartment is the eye. In some cases, the sterile anatomic compartment is the ear. In some cases, the sterile anatomic compartment is the brain.

In some cases, implantation into the body comprises implantation into a nonsterile anatomic compartment. In some cases, the nonsterile anatomic compartment is selected from the vagina, the rectum, and the nasal cavity. In some cases, the nonsterile anatomic compartment is the vagina. In some cases, the nonsterile anatomic compartment is the rectum. In some cases, the nonsterile anatomic compartment is the nasal cavity.

Provided herein are devices comprising a shape adapted to be disposed within the body of a patient. In some cases, the device is capsule-shaped.

### Use and Applications of the Device

Provided herein are methods of delivering one or more APIs to a subject in need thereof, the method comprising providing a drug delivery device disclosed herein to the subject. In some cases, providing the drug delivery device comprises implanting the drug delivery device into the subject's body. Also provided are method of treating or preventing a disease or disorder in a subject, the method comprising providing a drug delivery device described herein to the subject. In some cases, the disease or disorder is an infectious disease, a transplant graft rejection, a condition indicating hormonal therapy, a physiological or pathophysiological application, diabetes mellitus, an allergy or hypersensitivity, an autoimmune disorder, cancer, a hematological disease, a musculoskeletal disorder, a psychological or neurological disorder, a genetic disease, or a veterinary condition. In some cases, the disease or disorder is an infectious disease. In some cases, the infectious disease is HIV.

One purpose of the drug delivery devices described herein is to deliver one or more APIs to a body compartment for the purposes of treating, preventing, reducing the likelihood of having, reducing the severity of and/or slowing the progression of a medical condition in a subject, termed “application” hereunder. In some cases, the anatomic compartment is the vagina. In other cases, the target body compartment is systemic circulation. The purpose is augmented by the associated intent of increasing patient compliance by reducing problems in adherence to treatment and prevention associated with more frequent dosing regimens. Consequently, the disclosure relates to a plurality of applications. Illustrative, non-restrictive examples of such applications are provided below in summary form. Based on these examples, one skilled in the art could adapt the disclosed technology to other applications. One skilled in the art would recognize whether such applications involve topical API delivery (e.g., certain vaginal implant devices such as IVRs; microarray patches for transdermal or ocular delivery) or systemic API delivery (e.g., subdermal or intramuscular implant devices; subdermal or intramuscular injection of particle suspensions).

### Infectious Diseases, Including Multiple, Overlapping Infections:

In some cases, a patient in need of treatment for a disease or disorder disclosed herein, such as an infectious disease, is symptomatic for the disease or disorder. In some cases, a patient in need of treatment for a disease or disorder disclosed herein, such as an infectious disease, is asymptomatic for the disease or disorder. A patient in need of treatment for a disease or disorder disclosed herein can be identified by a skilled practitioner, such as without limitation, a medical doctor or a nurse.

HIV prevention using one or more one or more suitable peptide conjugates with antiretroviral agents, including biologics, and/or one or more vaccines and/or adjuvants delivered from a drug delivery device(i.e., an implant or injectable formulation, as described herein); and treatment, using one or more suitable peptide conjugates with antiretroviral agents, including biologics, delivered from the drug delivery device.

Sexually transmitted infections (STIs), including but not limited to prevention or treatment, both active and chronic active, with peptide conjugates with one or more suitable antimicrobial agents delivered from a drug delivery device. Illustrative, but not limiting examples of STIs include: gonorrhea, chlamydia, lymphogranuloma venereum, syphilis, including multidrug-resistant (MDR) organisms, hepatitis C virus, and herpes simplex virus.

Bacterial vaginosis (BV), as well as other microbial dysbiotic vaginal states, including but not limited to prevention or treatment, both active and chronic active, with one or more suitable APIs delivered from a drug delivery device.

Hepatitis B virus (HBV) prevention or treatment, both active and chronic active, with one or more suitable APIs delivered from a drug delivery device.

Herpes simplex virus (HSV) and varicella-zoster virus (shingles) Zoster/Shingles, prevention or treatment, both active and chronic active, with one or more suitable APIs delivered from a drug delivery device.

Cytomegalovirus (CMV) and congenital CMV infection, prevention or treatment, both active and chronic active, with one or more suitable APIs delivered from a drug delivery device.

Malaria, prevention or treatment, both active and chronic active, with one or more suitable APIs delivered from a drug delivery device.

Tuberculosis, including multidrug-resistant (MDR) and extensively drug-resistant (XDR) tuberculosis, prevention or treatment, both active and chronic active, with one or more suitable APIs delivered from a drug delivery device.

Acne, treatment or management with one or more suitable APIs delivered from a drug delivery device.

Respiratory viral infections, prevention or treatment, including, but not limited to influenza viruses and coronaviruses, for example SARS-CoV-2.

Influenza spreads around the world in seasonal epidemics, resulting in the deaths of hundreds of thousands annually-millions in pandemic years. For example, three influenza pandemics occurred in the 20th century and killed tens of millions of people, with each of these pandemics being caused by the appearance of a new strain of the virus in humans. Often, these new strains result from the spread of an existing influenza virus to humans from other animal species. Influenza viruses are RNA viruses of the family Orthomyxoviridae, which comprises five genera: Influenza virus A, Influenza virus B, Influenza virus C, Isavirus and Thogoto virus. The influenza A virus can be subdivided into different serotypes based on the antibody response to these viruses. The serotypes that have been confirmed in humans, ordered by the number of known human pandemic deaths, are: H1N1 (which caused Spanish influenza in 1918), H2N2 (which caused Asian Influenza in 1957), H3N2 (which caused Hong Kong Flu in 1968), H5N1 (a pandemic threat in the 2007-08 influenza season), H7N7 (which has unusual zoonotic potential), H1N2 (endemic in humans and pigs), H9N2, H7N2, H7N3 and H10N7. Influenza B causes seasonal flu and influenza C causes local epidemics, and both influenza B and C are less common than influenza A.

Coronaviruses are a family of common viruses that cause a range of illnesses in humans from the common cold to severe acute respiratory syndrome (SARS). Coronaviruses can also cause a number of diseases in animals. Coronaviruses are enveloped, positive-stranded RNA viruses whose name derives from their characteristic crown-like appearance in electron micrographs. Coronaviruses are classified as a family within the Nidovirales order, viruses that replicate using a nested set of mRNAs. The coronavirus subfamily is further classified into four genera: alpha, beta, gamma, and delta coronaviruses. The human coronaviruses (HCoVs) are in two of these genera: alpha coronaviruses (including HCoV-229E and HCoV-NL63) and beta coronaviruses (including HCoV-HKU1, HCoV-OC43, Middle East respiratory syndrome coronavirus (MERS-CoV), the severe acute respiratory syndrome coronavirus (SARS-CoV), and SARS-CoV-2.

### Transplants—Graft Rejection:

Chronic immune-suppressive post-transplant therapy with one or more suitable APIs delivered from a drug delivery device.

### Hormonal Therapy:

Contraception, including estrogens and progestins, with one or more suitable APIs delivered from a drug delivery device.

Hormone replacement, with one or more suitable APIs delivered from a drug delivery device.

Testosterone replacement, with one or more suitable APIs delivered from a drug delivery device.

Thyroid replacement/blockers, with one or more suitable APIs delivered from a drug delivery device.

Hormonal treatment to regulate triglycerides (TGs) using one or more suitable APIs delivered from a drug delivery device.

Chronic pharmacologic support for all transgender individuals (all stages from cis-trans), using one or more suitable APIs delivered from a drug delivery device.

### Physiology and Pathophysiology:

Gastrointestinal (GI) applications, with one or more suitable agents delivered from the implant, including, but not limited to the treatment/management of diarrhea, pancreatic insufficiency, cirrhosis, fibrosis in all organs; GI organs-related parasitic diseases, gastroesophageal reflux disease (GERD).

Cardiovascular applications, with one or more suitable APIs delivered from a drug delivery device, including, but not limited to the treatment/management of hypertension (HTN) using, for example, statins or equivalent, cerebral/peripheral vascular disease, stroke/emboli/arrhythmias/deep venous thrombosis (DVT) using, for example anticoagulants and anti-atherosclerotic cardiovascular disease (ASCVD) medications, and congestive heart failure (CHF) using for example β-blockers, ACE inhibitors, and angiotensin receptor blockers.

Pulmonary applications, with one or more suitable APIs delivered from a drug delivery device, including, but not limited to the treatment/management of sleep apnea, asthma, longer-term pneumonia treatment, pulmonary HTN, fibrosis, and pneumonitis.

Bone applications, with one or more suitable APIs delivered from a drug delivery device, including, but not limited to the treatment/management of chronic pain (joints as well as bone including sternal), osteomyelitis, osteopenia, cancer, idiopathic chronic pain, and gout.

Urology applications, with one or more suitable APIs delivered from a drug delivery device, including, but not limited to the treatment/management of benign prostatic hyperplasia (BPH), bladder cancer, chronic infection (entire urologic system), chronic cystitis, prostatitis.

Ophthalmology applications, with one or more suitable APIs delivered from a drug delivery device, including, but not limited to the treatment/management of glaucoma, ocular infections.

Cholesterol management, with one or more suitable APIs delivered from a drug delivery device.

Metabolic applications, with one or more suitable APIs delivered from a drug delivery device, including, but not limited to the treatment/management of weight gain, weight loss, obesity, malnutrition (replacement), osteopenia, Vitamin deficiency (B vitamins/D), folate, and smoking/drug reduction/cessation.

### Diabetes Mellitus:

Treatment and management of diabetes (type 1 and 2), with one or more suitable APIs delivered from a drug delivery device.

Allergies and Hypersensitivities, with “Desensitization”, Often Need Low-Dose Repeated Exposure:

TYPES: Type I (IgE mediated reactions), Type II (antibody mediated cytotoxicity reactions), Type Ill (immune complex-mediated reactions), and Type IV for delayed type hypersensitivity (154), with one or more suitable APIs delivered from a drug delivery device.

Hypersensitivity reactions (HSRs), with one or more suitable APIs delivered from a drug delivery device.

Antibiotics, biologics (drug and antibody portion), chemotherapy (e.g., platins), progesterone, as well as other treatments known in the art and described in (154), with one or more suitable APIs delivered from a drug delivery device.

Food allergies (e.g., nuts, shellfish) with one or more suitable APIs delivered from a drug delivery device.

Allergy medication dosing with one or more suitable APIs delivered from a drug delivery device, as an alternative to allergy shots, recommended for people with severe allergy symptoms who do not respond to usual medications; for people who have significant medication side effects from their medications; for people who find their lives disrupted by allergies/insect stings; or people for whom allergies might become life threatening: anaphylaxis.

### Autoimmune Disorders, Often Classified as Chronic Inflammatory Disorders:

Treatment and management of Crohn's disease and ulcerative colitis, with one or more suitable APIs delivered from a drug delivery device.

Rheumatoid arthritis (RA) treatment and management with one or more suitable APIs delivered from a drug delivery device.

Multiple sclerosis (MS) treatment and management with one or more suitable APIs delivered from a drug delivery device.

Psoriasis treatment and management with one or more suitable APIs delivered from a drug delivery device.

Lupus treatment and management with one or more suitable APIs delivered from a drug delivery device.

Autoimmune thyroiditis treatment and management with one or more suitable APIs delivered from a drug delivery device.

### Oncology:

Chemotherapy and targeted therapy (e.g., Ig) chronic or sub-chronic cancer management with one or more suitable APIs delivered from a drug delivery device.

### Hematologic Diseases:

Treatment/management of Hemophilia A with one or more suitable APIs (e.g., Factor VIII orthologs) delivered from a drug delivery device.

Administration of anticoagulants and/or antiplatelet therapy with one or more suitable APIs delivered from a drug delivery device.

Treatment/management of leukemia/lymphoma and bone marrow transplant (MBT) therapies with one or more suitable APIs delivered from a drug delivery device.

Iron replacement therapy with one or more suitable APIs delivered from a drug delivery device.

### Musculoskeletal Applications:

One or more anti-inflammatory APIs (e.g., NSAIDS) delivered from a drug delivery device.

Opioids addiction/pain management with one or more suitable APIs delivered from a drug delivery device.

Hypertrophic fibrosis/scar tissue.

### Psychological and Neurologic Disorders:

Treatment and management of depression with one or more suitable agents APIs delivered from a drug delivery device.

Treatment and management of schizophrenia, and related, with one or more suitable APIs delivered from a drug delivery device.

Treatment and management of bipolar disorders with one or more suitable APIs delivered from a drug delivery device.

Treatment and management of dysthymic disorders with one or more suitable APIs delivered from a drug delivery device.

Treatment and management of seizure control with one or more suitable APIs delivered from a drug delivery device.

Treatment and management of ADD/ADHD and hyperactivity disorders with one or more suitable APIs delivered from a drug delivery device.

Treatment and management of behavioral/emotional secondary to early-onset (child/adolescent), substance use, physical, sexual, emotional abuse, PTSD, and anxiety with one or more suitable APIs delivered from a drug delivery device.

Treatment and management of seizures, including but not limited to epilepsy and traumatic brain injury with one or more suitable APIs delivered from a drug delivery device.

Treatment and management of Parkinson's disease with one or more suitable APIs delivered from a drug delivery device.

Treatment and management of Alzheimer's disease with one or more suitable APIs delivered from a drug delivery device.

### Genetic Diseases:

Treatment of congenital genetic deficiency diseases, including genetic excess diseases, with one or more suitable APIs delivered from a drug delivery device.

Treatment of primary immunodeficiencies (e.g., agammaglobulinemia, secretory IgA deficiency, sIgA deficiency) with one or more suitable APIs delivered from a drug delivery device.

Severe combined immunodeficiency (SCID) treated SCID with one or more suitable APIs delivered from a drug delivery device, including, but not limited to enzyme replacement therapy (ERT) with pegylated bovine ADA (PEG-ADA),

Muscular dystrophy treated and managed with one or more suitable APIs delivered from a drug delivery device.

Treatment or management of Duchenne's disease with one or more suitable APIs (e.g., eteplirsen) delivered from a drug delivery device.

Treatment or management of Pompe's disease with one or more suitable APIs delivered from a drug delivery device, including ERT such as intravenous administration of recombinant human acid α-glucosidase,

Treatment or management of Gaucher disease with one or more suitable APIs delivered from a drug delivery device.

Veterinary Applications involving all mammals, including, but not limited to dogs, cats, horses, pigs, sheep, goats, and cows.

In one embodiment, the delivery of APIs from a drug delivery device serves multiple purposes, where more than one application is targeted simultaneously. An example of such a multipurpose API delivery device involves the prevention of HIV infection, with the delivery of one or more antiretroviral agents, including peptide-drug conjugates, and contraception, with the delivery of one or more contraceptive agents. In another embodiment, the multipurpose API delivery device protects against multiple diseases using a single agent. The intravaginal delivery of a peptide against enveloped viruses, such as taken from the group described by Cheng et al. (155), incorporated by reference in its entirety, is used to prevent HIV and HSV infection, among other enveloped viruses. The peptide also can be combined with other agents (e.g., contraceptives and/or antiviral agents, and peptide conjugates thereof) in an IVR as a multipurpose prevention technology.

The disclosure also provides methods of delivering an API to subject via a device of the disclosure comprising a kernel comprising an excipient and an API (such as TAF) which is implanted in the subject. In some cases, the API is delivered with a consistent, sustained release profile. In some cases, the excipient is PEG or TEC.

Provided herein are methods of delivering one or more APIs to a patient in need thereof, comprising implanting a device disclosed herein into the patient's body. In some cases, the device delivers one or more APIs for 1 to 12 months. In some cases, delivers one or more APIs for 1 to 3 months. In some cases, the device delivers one or more APIs for 3 to 12 months. In some cases, the device delivers one or more APIs for 1, 2, 3, 4, 5, 6, 7, 8, 9, 10, 11, or 12 months. In some cases, the device delivers one API for 1 to 12 months. In some cases, delivers one API for 1 to 3 months. In some cases, the device delivers one API for 3 to 12 months. In some cases, the device delivers one API for 1, 2, 3, 4, 5, 6, 7, 8, 9, 10, 11, or 12 months. In some cases, the device delivers more than one API for 1 to 12 months. In some cases, delivers more than one API for 1 to 3 months. In some cases, the device delivers more than one API for 3 to 12 months. In some cases, the device delivers more than one API for 1, 2, 3, 4, 5, 6, 7, 8, 9, 10, 11, or 12 months.

In some cases, the API comprises a hydrophobic or hydrophilic drug. In some cases, the API comprises a hydrophobic drug. In some cases, the one or more drugs are hydrophobic. In some cases, the API comprises a hydrophilic drug. In some cases, the one or more drugs are hydrophilic In some cases, the API is tenofovir alafenamide, ivermectin, or a ROCK2 inhibitor. In some cases, the API is tenofovir alafenamide. In some cases, the API is ivermectin or a ROCK2 inhibitor. In some cases, the ROCK2 inhibitor is KD025 (Kadmon). In some cases, the one or more drugs are antiretrovirals, antimicrobial agents, antibacterial agents, antivirals, hormones, statins, β-blockers, ACE inhibitors, angiotensin receptor blockers, vitamins, steroids, biologics, anti-cancer drugs, allergy medications, anticoagulants, antiplatelet therapies, non-steroidal anti-inflammatory drugs, vaccines, or combinations thereof. In some cases, the one or more drugs are antiretrovirals. In some cases, the one or more drugs comprise zidovudine, cabotegravir, dapivirine, fluticasone propionate, chlordiazepoxide, haloperidol, indomethacin, prednisone, ethinyl estradiol, acyclovir, tenofovir, atenolol, aminoglycosides, exenatide acetate, leuprolide acetate, acetylsalicylic acid (aspirin), levodopa, remdesivir, acyclovir, ganciclovir, ribavirin, lamivudine, dolutegravir, chloroquine, hydroxychloroquine, azithromycin, lopinavir, ritonavir, EV-100, EV-200, EV-300, EV-400, KD025, tenofovir, emtricitabine, elvitegravir, lenacapavir, islatravir, C5A, VRC01, or combinations thereof. In some cases, the one or more drugs comprise tenofovir. In some cases, the one or more drugs comprise tenofovir alafenamide or tenofovir disoproxil fumarate. In some cases, the one or more drugs comprise tenofovir alafenamide. In some cases, the one or more drugs comprise tenofovir disoproxil fumarate.

### EQUIVALENTS

The present disclosure is not to be limited in terms of the particular embodiments described in this application, which are intended as illustrations of various aspects. Many modifications and variations can be made without departing from the spirit and scope of the disclosure, as will be apparent to those skilled in the art. Functionally equivalent methods, systems, and apparatus within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting. As will be understood by one skilled in the art, for any and all purposes, such as in terms of providing a written description, all ranges disclosed herein also encompass any and all possible subranges and combinations of subranges thereof.

As a person skilled in the art would readily know many changes can be made to the preferred embodiments without departing from the scope thereof. It is intended that all matter contained herein be considered illustrative of the disclosure and not in a limiting sense.

While various aspects and embodiments have been disclosed herein, other aspects and embodiments will be apparent to those skilled in the art. All references cited herein are incorporated by reference in their entireties.

### EXAMPLES

### Example 1—Illustrative Subdermal Implant Specifications for Tenofovir Alafenamide and HIV Prevention

The disclosed implant technology for the sustained, controlled delivery of APIs to systemic circulation is a platform technology and not directed at any specific API or application. The restrictions on the choice of API and, hence application, have been outlined under “Choice of API”. An illustrative, non-restrictive example of the interplay between API physical, chemical, and biological properties and implant characteristics is provided here.

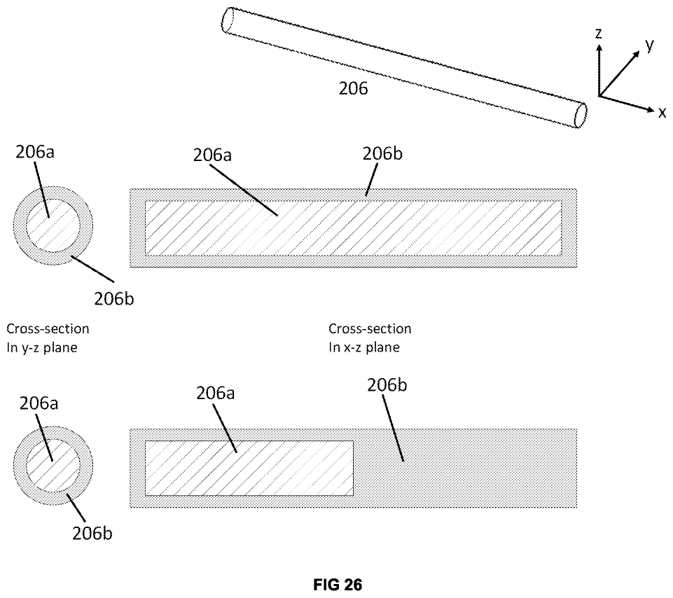

Tenofovir alafenamide (TAF) is a nucleoside reverse transcriptase inhibitor (NRTI) and a potent antiretroviral drug against HIV. Preclinical and clinical studies suggest that TAF delivered systemically could safely prevent HIV infection in uninfected individuals. It is believed by many in the art that steady-state concentrations of tenofovir diphosphate (TFV-DP), the active metabolite of TAF, in peripheral blood mononuclear cells (PBMCs) are predictive of efficacy in preventing sexual HIV transmission. It is further believed by many in the art that TFV-DP concentrations in PBMCs of 50 fmol per million cells is a good target concentration for effective HIV prevention. A simulation using physiologically based PK (PB-PK) modeling estimates that a linear, subcutaneous release of TAF at a rate of 0.5 mg d⁻¹ would lead to the above protective TFV-DP PBMC concentrations (165). Another study estimated that lower TAF release rates of ca. 0.3 mg d⁻¹ could be protective (166). Two subdermal implants of the design 206 shown in FIG. 26 (dimensions, 2.5 mm dia., 40 mm, length; volume, 196 mm³, TAF-peptide content, 75% w/v) each containing a TAF-peptide conjugate equivalent to 130 mg TAF and delivering at 0.25 mg d⁻¹ each, with zero order kinetics, could prevent HIV infection for up to one year.

### Example 2—Illustrative Subdermal Implant Specifications for Cabotegravir and HIV Prevention

The disclosed implant technology for the sustained, controlled delivery of APIs to systemic circulation is a platform technology and not directed at any specific API or application. The restrictions on the choice of API and, hence application, have been outlined under “Choice of API”. An illustrative, non-restrictive example of the interplay between API physical, chemical, and biological properties and implant characteristics is provided here.

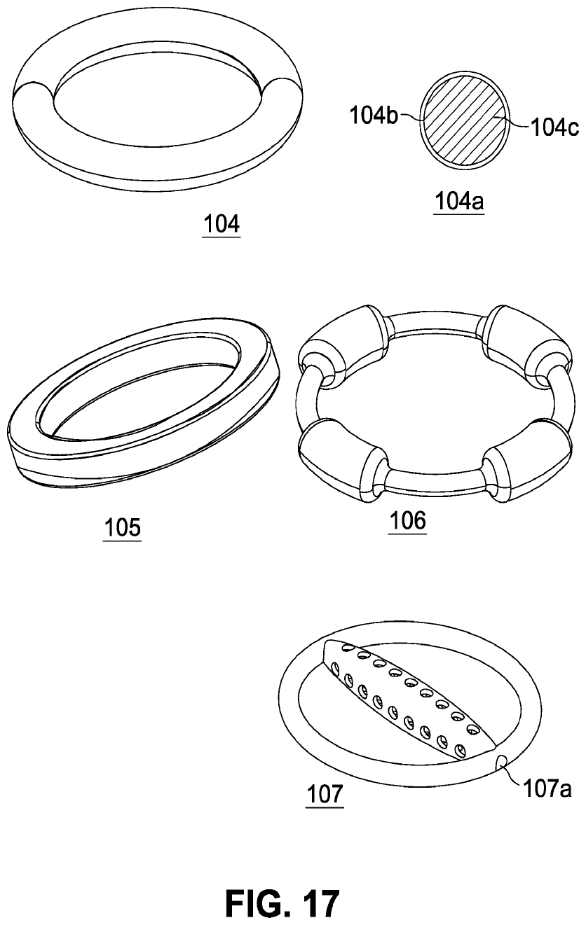

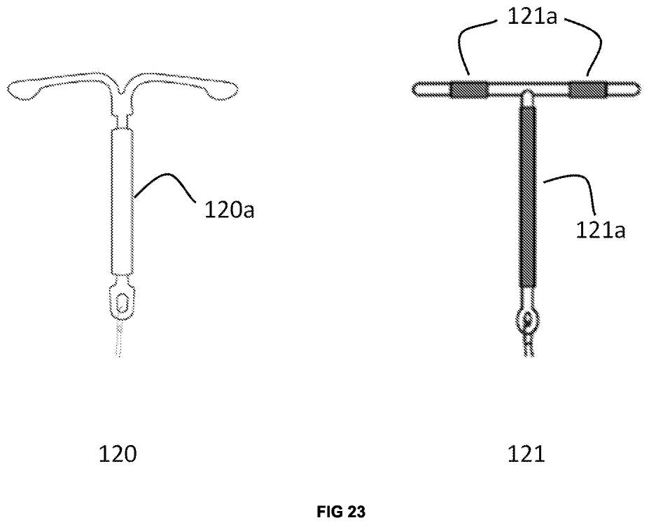

Cabotegravir (CAB) is a potent strand-transfer integrase inhibitor being developed for HIV treatment and prevention. It is believed by many in the art that steady-state plasma concentrations of CAB are predictive of efficacy in preventing sexual HIV transmission. It is further believed by many in the art that steady state plasma CAB concentrations of 0.66 μg mL⁻¹, or four times the protein adjusted IC₉₀ (PA-IC₉₀) concentration from non-human primate efficacy studies (167, 168), are a good target for effective HIV prevention. The target was adjusted subsequently based on human clinical PK data (169). A phase 2a trial evaluating an injectable, intramuscular, long-acting CAB formulation suggests that male and female participants dosed with 600 mg every 8 weeks met the targets of 80% and 95% of participants with trough concentrations above 4× and 1×PA-IC₉₀, respectively (170). Due to the tailing (i.e., non-steady state) PK profile of the injectable CAB formulation (169, 170), a lower dose or longer duration should be achievable from an CAB-peptide implant with linear in vivo drug release profiles. It is estimated that two subdermal or intramuscular implants of the geometry 102, Shown in FIG. 12, of design 202 shown in FIG. 24 (dimensions, 3.5 mm×2.5 mm×35 mm; volume, 306 mm³, CAB-peptide content, 85% w/v) each containing a CAB-peptide conjugate equivalent to 250 mg CAB and delivering at 2.2 mg d⁻¹ each, with zero order kinetics, could prevent HIV infection for up to three months.

### Example 3—Illustrative Drug Delivery Implant Specification Calculator

A non-limiting example of an algorithm for calculating the implant specifications for any application is given below.

$$V = \frac{( RR \times t )}{SF \times {m}_{f} \times ρ}$$

where,
V (mL) is the total implant volume (i.e., volume of single implant or sum of volumes of multiple implants),
RR (g d⁻¹) is the total API release rate of the implant(s). For nonlinear release rates, RR corresponds to the integral of cumulative drug release (y-axis) over time (x-axis) for the period of use, divided by t,
t (d) is the duration of use,
SF is a dimensionless scaling factor, typically between 0.50 and 0.99 to ensure that sufficient drug remains in the implant to maintain the target API delivery profile over the period of use,
m_{f} is the mass fraction of drug in the implant(s), typically between 0.25 and 0.95, to account for the presence of excipients,
ρ (g mL⁻¹) is the density of the implant(s).

The value of RR will be determined in part by the potency of the API and how efficiently it distributes to the target compartment(s) to achieve consistent pharmacologic efficacy. In many cases RR will need to be determined in preclinical studies and confirmed clinically.

### Example 4—TAF Microneedles as Porogens in PDMS

Tenofovir alafenamide free-base (TAF, 5.00 g) was added to toluene (200 mL) at 90° C. in a conical flask. To the cloudy solution was added more toluene (25 mL) with magnetic stirring. When the turbid suspension reached thermal equilibrium, it was filtered hot to afford a clear solution. The hot TAF solution was allowed to cool to room temperature overnight, followed by additional cooling at 4° C. resulting in copious needles depositing at the bottom of the flask. The solid was collected by filtration in vacuo, followed by washing with cold n-hexane, and drying under high vacuum to yield colorless TAF microneedles (4.35 g, 87%), typically 10-25 μm wide×250-450 μm long.

### Example 5—BSA Release Kinetics from ePTFE Implants

Implants were fabricated using an ePTFE tubing skin (ca. 20 mm length, 2.0 mm I.D., 0.18 mm wall thickness, ρ=0.84 g cm³) and filled with bovine serum albumin (BSA) as powders at 100% (i.e., in the absence of excipients) or at 50% w/w blended with D-(+)-trehalose (45% w/w) and L-histidine hydrochloride (5% w/w). In another group, the BSA (30% w/w) was blended with monoolein (Myverol 18-93K, 70% w/w) and added to the hollow tube as a paste. The implants contained between 10-20 mg BSA depending on the formulation. The ends of the tubes were sealed prior to conducting in vitro release studies in 20 mL of release media (1×PBS containing 0.1% solutol HS 15 and 0.01% sodium azide) at 37° C. in an orbital shaking incubator at 30 RPM. The analysis of BSA in the release media was carried out using the Bradford reagent (λₘₐₓ 595 nm). The 100% BSA powder did not appreciably release from the implants over 28 d while the implants that contained BSA formulated with D-(+)-trehalose and L-histidine hydrochloride released their BSA payload within 2 d. However, when co-formulated as a paste with monoolein, BSA released linearly from the implant over 8 d. The data were analyzed using a simple linear regression fit model (solid line) to afford a measured slope (release rate) of 1.7 mg d⁻¹ (R²=0.9800). It was unexpected and unpredictable that these three formulations would result in such dramatically different release profiles of a model biologic, BSA. The fact that the release of BSA, a highly water-soluble compound, from a monoolein paste and through an ePTFE skin was linear and controllable over one week is novel and unknown to one skilled in the art.

### Example 6—Cell Penetrating Peptide Conjugate of Lamivudine

The disclosed technology for the sustained, controlled delivery of APIs is not directed at any specific API or application. The restrictions on the choice of API and, hence application, have been outlined under “Choice of API”. An illustrative, non-restrictive example is provided here.

The antiviral agent lamivudine (3TC) is used to treat viral infections, including HIV and HBV, but is not sufficiently potent to be delivered efficiently using most systemic, long-acting formulation approaches.

In this example, 3TC is reacted with succinic anhydride to afford the corresponding succinate ester. The carboxylic acid group is coupled to the N-terminus of on-resin, side-chain protected octaarginine. Cleavage and purification of the resulting deprotected peptide-drug conjugate by HPLC yields the target compound.

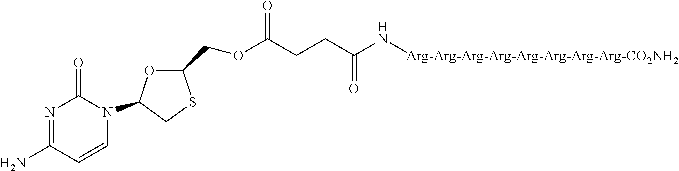

### Example 7—Cell Targeting Peptide Conjugate of Dolutegravir

The disclosed technology for the sustained, controlled delivery of APIs is not directed at any specific API or application. The restrictions on the choice of API and, hence application, have been outlined under “Choice of API”. An illustrative, non-restrictive example is provided here.

The antiretroviral agent dolutegravir (DTG) is used to treat HIV infections, but due to high protein binding it is not sufficiently potent to be delivered efficiently using most systemic, long-acting formulation approaches.

In this example, The N-terminus of on-resin, side-chain protected tuftsin (TKPR) is reacted with chloromethyl chloroformate to give the corresponding N-[(chloro-methyl)oxy]carbonyl derivative, which is reacted with DTG under phase transfer catalysis to yield the target peptide conjugate following resin cleavage and purification by HPLC.

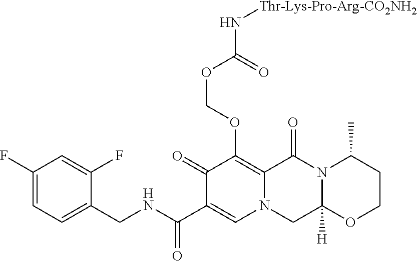

### Example 8—Cell Targeting Peptide Conjugate of Lamivudine

In this example, 3TC was reacted with succinic anhydride to afford the corresponding succinate ester. The carboxylic acid group was coupled to the N-terminus of on-resin, side-chain protected HIV-1 gp120 V3-A (RKSIHIGPGRAFYTTG). Cleavage of the resulting deprotected peptide-drug conjugate yielded the target compound with 69% purity by HPLC prior to purification. HRMS (ES-TOF) (m/z): calcd for C₉₁H₁₃₉N₂₉O₂₅S, 1036.0190 [M+2H]²⁺, 691.0151 [M+3H]³⁺, and 518.5132 [M+4H]⁴⁺; found, 1036.0178 (−1.16 ppm), 691.0137 (−2.03 ppm), and 518.5126 (−1.16 ppm).

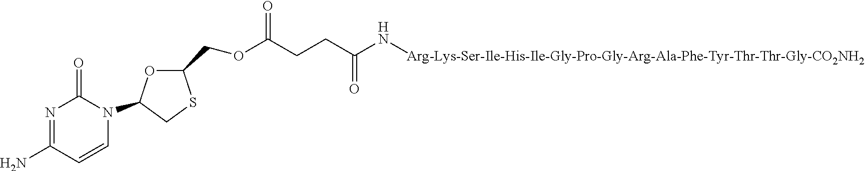

### Example 9—Screening Algorithm

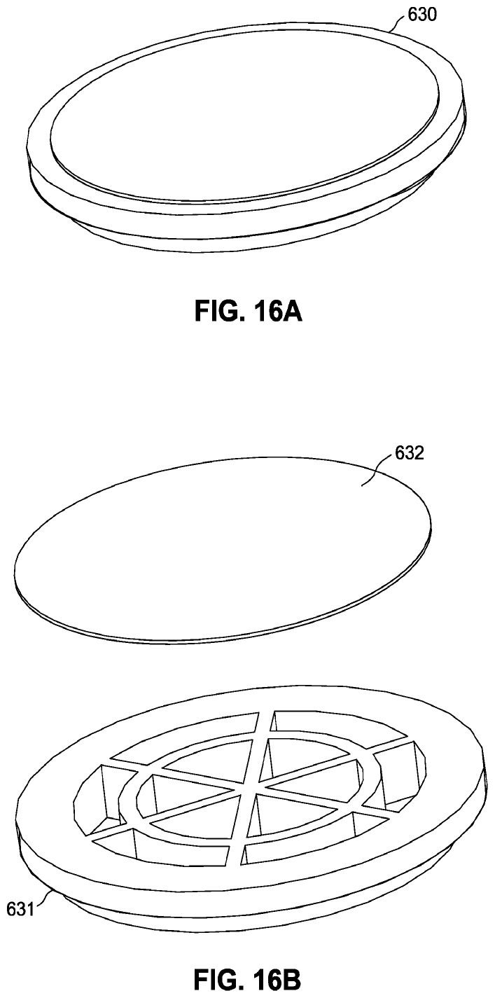

In cases when multiple drug-peptide conjugates are being screened during discovery, it may be beneficial to use a decision tree to select candidates for further testing. A nonlimiting example of a decision tree is shown in FIG. 11. The decision tree is used to score, rank, and down-select candidates based on clearly defined, quantitative rules informed by results from an evaluation pipeline, with the goal of selecting a lead candidate for formulation. In one nonlimiting example of a decision tree as shown in FIG. 11, ca. 28 compounds per ARV drug are used for in vitro screening studies. As shown in FIG. 11, efficacy is measured as a molar quantity, but the dose-capped cargo mass fraction taken up by the ARV drug relative to the prodrug is determined by their relative molecular weights. The larger the peptide molecular weight, the more diluted the drug implant content. This important consideration is factored into the selection process, by also considering the potency in mass concentration units. We recognize that the decision tree (FIG. 11) is a “living algorithm” and will evolve as the screening progresses.

### REFERENCES CITED

1. Landovitz, R. J., et al., N. Engl. J. Med. 2021, 385 (7), 595-608.
2. Marzinke, M. A., et al., J. Infect. Dis. 2021.
3. Kovarova, M., et al., Nat. Commun. 2018, 9 (1), 4156.
4. Min, S., et al., Antimicrob. Agents Chemother. 2010, 54 (1), 254-258.
5. Kandel, C. E., et al., Drug Des. Devel. Ther. 2015, 9, 3547-3555.
6. Somboonwit, C., et al., Expert Opin. Drug Metab. Toxicol. 2009, 5 (12), 1599-1606.
7. Kumar, M., et al., Expert Rev. Gastroenterol. Hepatol. 2008, 2 (4), 465-495.
8. Sanrame, C. N., et al., Mol. Pharm. 2014, 11 (10), 3617-3623.
9. Sillman, B., et al., Nat. Commun. 2018, 9 (1), 443.
10. Bundgaard, H., Designs of Prodrugs. Elsevier: New York, 1985; p 360.
11. Rautio, J., et al., Nat. Rev. Drug Discov. 2008, 7(3), 255-270.
12. Stella, V. J., J. Pharm. Sci. 2010, 99 (12), 4755-4765.
13. Rautio, J., et al., Nat. Rev. Drug Discov. 2018, 17 (8), 559-587.
14. Vives, E., et al., J. Biol. Chem. 1997, 272 (25), 16010-16017.
15. Regberg, J., et al., Pharmaceuticals 2012, 5(9), 991-1007.
16. Agrawal, P., et al., Nucleic Acids Res. 2016, 44 (D1), D1098-1103.
17. Shah, S. S., et al., Front. Chem. 2020, 8, 218.
18. Encyclopedia of Membranes. 1st ed.; Springer: Berlin, Heidelberg, Germany, 2016; p 2090.
19. Gu, Z., et al., ACS Nano 2013, 7 (8), 6758-6766.
20. Habibi, Y., et al., Chemical Reviews 2010, 110 (6), 3479-3500.
21. Voisin, H., et al., Nanomaterials 2017, 7(3), E57.
22. Delalat, B., et al., Nat. Commun. 2015, 6.
23. Uthappa, U. T., et al., J. Control. Release 2018, Epub ahead of print May 14.
24. Kuo, S.-H., et al. U.S. Pat. No. 7,842,303 B2, Nov. 30, 2010.
25. Welton, T., Chem. Rev. 1999, 99 (8), 2071-2084.
26. Hallett, J. P., et al., Chem. Rev. 2011, 111 (5), 3508-3576.
27. Shamshina, J. L., et al., Expert Opin. Drug Deliv. 2013, 10 (10), 1367-1381.
28. Dunn, R. L., et al. U.S. Pat. No. 4,938,763A, Ju. 3, 1990.
29. Royals, M. A., et al., J. Biomed. Mater. Res. 1999, 45 (3), 231-239.
30. Ravivarapu, H. B., et al., J. Pharm. Sci. 2000, 89 (6), 732-741.
31. Ravivarapu, H. B., et al., Int. J. Pharm. 2000, 194 (2), 181-191.
32. Wu, W. Q., et al., Mol. Pharm. 2014, 11 (10), 3378-3385.
33. Han, L., et al., Biomaterials 2016, 105, 185-194.
34. Wang, M., et al., J. Control. Release 2016, 230, 45-56.
35. Xiang, N. X., et al., J. Pharm. Sci. 2016, 105 (3), 1148-1155.
36. Khan, U. A., et al., Drug Des. Devel. Ther. 2020, 14, 2237-2247.
37. Milak, S., et al., Int. J. Pharm. 2015, 478 (2), 569-587.
38. Alsberg, E., et al., Journal of Dental Research 2001, 80 (11), 2025-2029.
39. Sun, J. Y., et al., Nature 2012, 489 (7414), 133-136.
40. Yang, Y., et al., Chemical Society Reviews 2013, 42 (17), 7446-7467.
41. Appel, E. A., et al., Nat. Commun. 2015, 6, 6295.
42. Fenton, O. S., et al., Biomacromolecules 2019, 20 (12), 4430-4436.
43. Steele, A. N., et al., Adv. Healthc. Mater. 2019, 8 (5), e1801147.
44. Liu, F., et al., Progress in Polymer Science 2010, 35 (1-2), 3-23.
45. Wei, M. L., et al., Polymer Chemistry 2017, 8 (1), 127-143.
46. Singh, S., et al., Int. J. Pharm. 2007, 341 (1-2), 68-77.
47. Rey-Rico, A., et al., Int. J. Mol. Sci. 2018, 19 (3).
48. Chakraborty, S., et al., Adv. Drug Deliv. Rev. 2009, 61 (12), 1043-1054.
49. Ball, C., et al., PLoS One 2012, 7 (11), e49792.
50. Blakney, A. K., et al., Antiviral Res. 2013, 100, S9-S16.
51. Ball, C., et al., Antimicrob. Agents Chemother. 2014, 58 (8), 4855-4865.
52. Krogstad, E. A., et al., Int. J. Pharm. 2014, 475(1-2), 282-291.
53. Chou, S. F., et al., J. Control. Release 2015, 220, 584-591.
54. Ball, C., et al., Mater. Sci. Eng. C-Mater. Biol. Appl. 2016, 63, 117-124.
55. Blakney, A. K., et al., ACS Biomater. Sci. Eng. 2016, 2 (9), 1595-1607.
56. Carson, D., et al., Pharm. Res. 2016, 33 (1), 125-136.
57. Leach, M. K., et al., J. Vis. Exp. 2011, 47, e2494.
58. Wu, Y. H., et al., E-Polymers 2017, 17 (1), 39-44.
59. Huang, Z. X., et al., Mater. Manuf. Process. 2018, 33 (2), 202-219.
60. Jiang, H. L., et al., J. Control. Release 2014, 193, 296-303.
61. Yu, D. G., et al., Chem. Commun. 2017, 53 (33), 4542-4545.
62. Yoo, H. S., et al., Adv. Drug Deliv. Rev. 2009, 61 (12), 1033-1042.
63. Quiros, J., et al., Polymer Reviews 2016, 56 (4), 631-667.
64. Katta, P., et al., Nano Lett. 2004, 4 (11), 2215-2218.
65. Sundaray, B., et al., Appl. Phys. Lett. 2004, 84 (7), 1222-1224.
66. Dalton, P. D., et al., Polymer 2005, 46 (3), 611-614.
67. Liu, L. Q., et al., Appl. Phys. Lett. 2007, 90 (8).
68. Zheng, J., et al., Nanoscale Res. Lett. 2015, 10 (1), 475.
69. Badrossamay, M. R., et al., Nano Lett. 2010, 10 (6), 2257-2261.
70. Mellado, P., et al., Appl. Phys. Lett. 2011, 99 (20), 203107.
71. Wang, L., et al., Microelectron. Eng. 2011, 88 (8), 1718-1721.
72. Gonzalez, G. M., et al., Macromol. Mater. Eng. 2017, 302 (1), 1600365.
73. Kroschwitz, J. I., Encyclopedia of Polymer Science and Engineering. 2nd ed.; John Wiley & Sons: New York, 1986; Vol. 6.
74. Blades, H. U.S. Pat. No. 3,767,756, Oct. 23, 1973.
75. Park, S. K., et al., Fibers Polym. 2000, 1 (2), 92-96.
76. Vallet-Regi, M., et al., Eur. J. Inorg. Chem. 2003, (6), 1029-1042.
77. Ahola, M., et al., Int. J. Pharm. 2000, 195 (1-2), 219-227.
78. Giri, S., et al., Nanomed.-Nanotechnol. Biol. Med. 2007, 2 (1), 99-111.
79. Kaity, S., et al., J. Adv. Pharm. Technol. Res. 2010, 1 (3), 283-290.
80. Choi, S. J., et al., ACS Appl. Mater. Interfaces 2011, 3 (12), 4552-4556.
81. Yu, C. L., et al., Adv. Mater. Interfaces 2017, 4 (3).
82. Calcagnile, P., et al., ACS Nano 2012, 6 (6), 5413-5419.
83. Tejashri, G., et al., Acta Pharm. 2013, 63 (3), 335-358.
84. Deuber, F., et al., Chemistry Select 2016, 1 (18), 5595-5598.
85. Deuber, F., et al., ACS Appl. Mater. Interfaces 2018, 10 (10), 9069-9076.
86. Amin Yavari, S., et al., Biomaterials 2014, 35 (24), 6172-6181.
87. Li, X., et al., Regen. Biomater. 2015, 2 (3), 221-228.
88. Yasenchuk, Y., et al., Materials 2019, 12 (15).
89. Heikkinen, M. S. A., et al., J. Aerosol Sci. 2000, 31 (6), 721-738.
90. Ou, Q., et al., Aerosol Sci. Technol. 2017, 51 (11), 1303-1312.
91. Uhlmann, E., et al., Procedia CIRP 2015, 35, 55-60.
92. Grummon, D. S., et al., Appl. Phys. Lett. 2003, 82 (16), 2727-2729.
93. Lindahl, C., et al., ISRN Biomater. 2013, 2013, 205601.
94. Gultepe, E., et al., Adv. Drug Deliv. Rev. 2010, 62 (3), 305-315.
95. Badar, M., et al., J. Biomed. Mater. Res. A 2015, 103 (6), 2141-2149.
96. Shi, J. R., et al., J. Mat. Chem. B 2016, 4 (46), 7415-7422.
97. Si, P. X., et al., Polym. Adv. Technol. 2015, 26 (9), 1091-1096.
98. Priyadarshani, J., et al., A/P Conf. Proc. 2020, 2270, 020004.
99. Gore, R. W. U.S. Pat. No. 3,953,566, Apr. 27, 1976.
100. Shim, J. H., et al., Biofabrication 2011, 3 (3), 034102.
101. Bernards, D. A., et al., Advanced Materials 2010, 22 (21), 2358-2362.
102. Koch, B., et al., Materials 2016, 9 (8).
103. Baeten, J. M., et al., N. Engl. J. Med. 2016, 375, 2121-2132.
104. Nel, A., et al., N. Engl. J. Med. 2016, 375 (22), 2133-2143.
105. Kirschman, J. L., et al., Nucleic Acids Res. 2017, 45 (12).
106. Tiwari, P. M., et al., Nat. Commun. 2018, 9.
107. Loomis, K. H., et al., Bioconjugate Chem. 2018, 29 (9), 3072-3083.
108. Zhang, L., et al., Acs Biomaterials Science & Engineering 2017, 3 (8), 1654-1665.
109. Chang, L., et al., J. Pharm. Sci. 2009, 98 (9), 2886-2908.
110. Mensink, M. A., et al., Eur. J. Pharm. Biopharm. 2017, 114, 288-295.
111. Forney-Stevens, K. M., et al., J. Pharm. Sci. 2015, 105, 697-704.
112. Kuzma, P., et al. U.S. Pat. No. 7,858,110 B2, Dec. 28, 2010.
113. Cima, M. J., et al. U.S. Pat. No. 9,586,035 B2, Dec. 11, 2008.
114. Conrad, K., et al., Arch. Facial Plast. Surg. 2008, 10 (4), 224-231.
115. Zelken, J. A., et al., Ann. Plast. Surg. 2017, 78 (2), 131-137.
116. Conrad, K., et al., J. Otolaryngol. 1992, 21 (3), 218-222.
117. Kastellorizios, M., et al., Foreign Body Reaction to Subcutaneous Implants. In Immune Responses to Biosurfaces: Mechanisms and Therapeutic Interventions, Lambris, J. D.; Ekdahl, K. N.; Ricklin, D.; Nilsson, B., Eds. 2015; Vol. 865, pp 93-108.
118. Vegas, A. J., et al., Nat. Biotechnol. 2016, 34 (3), 345-352.
119. Manavitehrani, I., et al., Polymers 2016, 8 (1).
120. Teo, A. J. T., et al., ACS Biomater. Sci. Eng. 2016, 2 (4), 454-472.
121. Donnelly, R. F., et al., Drug Discov. Today 2018, 23 (5), 1026-1033.
122. Alkilani, A. Z., et al., Pharmaceutics 2022, 14 (6).
123. Singh, R. R. T., et al., Expert Opin. Drug Deliv. 2017, 14 (4), 525-537.
124. Wu, Y., et al., Drug Deliv. Transl. Res. 2022.
125. DiCesare, P., et al. U.S. Pat. Appl. US 2016/0213904 A1, Aug. 5, 2010.
126. Blanc, D., et al. U.S. Pat. No. 9,056,953 B2, Jun. 16, 2015.
127. Escale, P., et al., Eur. Polym. J. 2012, 48 (6), 1001-1025.
128. Zhu, N., et al., Biofabrication of Tissue Scaffolds. In Advances in Biomaterials Science and Biomedical Applications, Pignatello, R., Ed. InTech: Rijeka, Croatia, 2013; pp 315-328.
129. Brazinsky, I., et al. U.S. Pat. No. 4,138,459 A, Feb. 6, 2979.
130. Oh, I.-H., et al., Scr. Mater. 2003, 49, 1197-1202.
131. Bansiddhi, A., et al., Acta Biomater. 2008, 4 (4), 773-782.
132. Mansourighasri, A., et al., J. Mater. Process. Technol. 2012, 212 (1), 83-89.
133. Wen, C. E., et al., Scr. Mater. 2001, 45(10), 1147-1153.
134. Kotan, G., et al., Turkish J. Eng. Env. Sci. 2007, 32, 149-156.
135. Niu, W., et al., Mater. Sci. Eng. A 2009, 506 (1), 148-151.
136. Ryan, G., et al., Biomaterials 2006, 27(13), 2651-26; Ryan et al., Biomaterials 2006, 27 (13), 2651-267070.
137. Jonathan, G., et al., Int. J. Pharm. 2016, 499 (1-2), 376-394.
138. Liaw, C. Y., et al., Biofabrication 2017, 9 (2).
139. Dickens Jr., E. D., et al. U.S. Pat. No. 5,648,450, Jul. 15, 1997.
140. Bandyopadhyay, A., et al., Ann. Biomed. Eng. 2017, 45 (1), 249-260.
141. Mullen, L., et al., J. Biomed. Mater. Res. B Appl. Biomater. 2009, 89 (2), 325-34.
142. Vaucher, S., et al., Phys. Status Solidi 2003, 199 (3), R11-R13.
143. Harrysson, O. L. A., et al., Direct Fabrication of Custom Orthopedic Implants Using Electron Beam Melting Technology. In Advanced Manufacturing Technology for Medical Applications, 2005; pp 191-206.
144. Lin, C. Y., et al., J. Biomed. Mater. Res. A 2007, 83 (2), 272-279.
145. Li, J. P., et al., Journal of materials science. Materials in medicine 2005, 16 (12),
1159-1163.
146. Li, J. P., et al., J. Biomed. Mater. Res. A 2005, 73 (2), 223-233.
147. Li, J. P., et al., Biomaterials 2006, 27 (8), 1223-1235.
148. Kummailil, J., et al., J. Manuf. Process. 2005, 7 (1), 42-50.
149. Kraibuhler, H., et al. U.S. Pat. No. 9,889,604, Feb. 13, 2018.
150. Vidin, E., et al., Contraception 2007, 76 (1), 35-39.
151. Persaud, T., et al., Eur. Radiol. 2008, 18 (11), 2582-2585.
152. Mascarenhas, L., Contraception 1998, 58 (6), 79S-83S.
153. Nguyen, T.-T., et al., Contraceptive Procedures: Subdermal Contraceptive Implants. In A Practical Guide to Office Gynecologic Procedures, 2nd ed. ed.; Blumenthal, P. D.; Berek, J. S., Eds. Lippincott Williams & Wilkins: Philadelphia, PA, 2013; pp 145-154.
154. de Las Vecillas Sanchez, L., et al., Int. J. Mol. Sci. 2017, 18 (6), E1316.
155. Cheng, G., et al., Proc. Natl. Acad. Sci. U.S.A. 2008, 105 (8), 3088-3093.
156. Moss, J. A., et al., Microbicide Vaginal Rings. In Drug Delivery and Development of Anti-HIV Microbicides, das Neves, J.; Sarmento, B., Eds. Pan Stanford Publishing: Singapore, 2014; pp 221-290.
157. Groenewegen, R. J. J. U.S. Pat. No. 5,989,581, Apr. 8, 1998.
158. Smith, J. M., et al., Proc. Natl. Acad. Sci. USA. 2013, 110 (40), 16145-16150.
159. Clark, J. T., et al., PLoS One 2014, 9 (3).
160. Woolfson, A. D., et al. U.S. Pat. No. 8,962,010 B2, Jun. 26, 2008.
161. Moss, J. A., et al. U.S. Patent 61/520,152, Jun. 6, 2011.
162. Baum, M. M., et al., J. Pharm. Sci. 2012, 101 (8), 2833-2843.
163. Benhabbour, S. R., et al. U.S. Pat. Appl. 2019/0091141 A1, Mar. 23, 2016.
164. Welsh, N. R., et al., Int. J. Pharm. 2019, Epub ahead of print October 21.
165. Kumar, R., et al. In In Silico Simulation of Long-acting Tenofovir Alafenamide Subcutaneous Implant, 2019 Conference on Retroviruses and Opportunistic Infections (CROI), Seattle, WA, Mar. 4-7, 2019; CROI, Alexandria, VA: Seattle, WA, 2019; p Abstract Number 487.
166. Gunawardana, M., et al., Antimicrob. Agents Chemother. 2015, 59 (7), 3913-3919.
167. Andrews, C. D., et al., Science 2014, 343 (6175), 1151-1154.
168. Andrews, C. D., et al., Sci. Transl. Med. 2015, 7 (270).
169. Markowitz, M., et al., Lancet HIV 2017, 4 (8), E331-E340.
170. Landovitz, R. J., et al., PLoS Med. 2018, 15 (11).

What is claimed is:

1. A drug delivery device comprising one or more peptide-drug conjugates, wherein the device comprises
(a) one or more kernels comprising the peptide-drug conjugate; and
(b) one or more skins comprising a continuous membrane;
wherein the one or more kernels and/or the skin comprises defined pores which are not produced mechanically, and
wherein the peptide-drug conjugate comprises a cell-penetrating peptide or cell-targeting peptide covalently linked to one or more drugs via one or more linkers.

2. The drug delivery device of claim 1, wherein the peptide comprises a cell-penetrating peptide.

3. The drug delivery device of claim 1, wherein the peptide comprises a cell-targeting peptide.

4. The drug delivery device of claim 3, wherein the peptide is an immunopotentiating peptide.

5. The drug delivery device of any one of claims 1 to 4, wherein the peptide is linear.

6. The drug delivery device of any one of claims 1 to 4, wherein the peptide is cyclic.

7. The drug delivery device of any one of claims 1 to 6, wherein the peptide is selected from a peptide in Table A.

8. The drug delivery device of any one of claims 1 to 7, wherein the one or more linkers are covalently linked to a terminal residue of the peptide.

9. The drug delivery device of claim 8, wherein the terminal residue is an N-terminal residue.

10. The drug delivery device of claim 8, wherein the terminal residue is a C-terminal residue.

11. The drug delivery device of any one of claims 1 to 7, wherein the one or more linkers are covalently linked to a non-terminal residue of the peptide.

12. The drug delivery device of claim 11, wherein the one or more linkers are covalently linked to a side chain of the non-terminal residue.

13. The drug delivery device of claim 11 or 12, wherein the non-terminal residue is a lysine, a cysteine, an aspartic acid, or a glutamic acid residue.

14. The drug delivery device of any one of claims 1 to 13, wherein the one or more linkers are (C₁-C₈)alkyl, (C₂-C₈)alkenyl, (C₂-C₈)alkynyl, (C₃-C₁₀)cycloalkyl, (C₆-C₁₀)aryl, (C₂-C₈)heteroalkyl, or (C₂-C₈)heteroaryl moieties; wherein alkyl, alkenyl, alkynyl, cycloalkyl, aryl, or heteroaryl is optionally substituted by 1 to 3 (C₁-C₆)alkyl, 1-4 halogens, (C₁-C₄)alkoxy, (C₁-C₄)alkoxycarbonyl, hydroxy, amino, (C₁-C₄)alkylamino, (C₁-C₄)dialkylamino, (C₃-C₁₀)cycloalkyl, (C₁-C₆)alkylcarbonyloxy, (C₁-C₆)alkylcarbonylamido, (C₁-C₄)alkylamidocarbonyl, (C₁-C₄)dialkylamidocarbonyl, nitro, cyano, (C₁-C₄)alkylimino, mercapto, or (C₁-C₄)alkylmercapto moieties.

15. The drug delivery device of any one of claims 1 to 13, wherein the one or more linkers have the structure:
—C(O)-J-C(O)—K—C(O)—,
wherein K is selected from —C₁-C₁₀alkylene-, —(CH₂),A-(CH₂)ₜ— and Q; —C(O)-J- is absent, an amino acid residue or a peptide of 2 to 10 amino acid residues,
wherein the —C(O)— is derived from the carboxy terminal of the amino acid or peptide;
A is —O—, —S—, —NR₁—, —N⁺(R₁)₂—, —S—S—, —[OCH₂CH₂]ᵣ—O—, -L-, or —O—Y—O—;
Q is L or —Z═N—NH—S(O)ᵥ-L-;
L is cycloalkyl, heterocycloalkyl, aryl, or heteroaryl;
Z is —(CH₂)_{w}—C(CH₃)═, —(CH₂)_{w}CH═, cycloalkyl, or heterocycloalkyl;
R₁ is hydrogen or C₁-C₄alkyl;
s and t are independently an integer selected from 1 and 2;
r is an integer selected from 1, 2, and 3;
v is an integer selected from 0, 1, and 2; and
w is an integer selected from 1, 2, 3, and 4.

16. The drug delivery device of any one of claims 1 to 13, wherein the one or more linkers comprise two or more amino acids.

17. The drug delivery device of claim 16, wherein one or more linkers are a peptide.

18. The drug delivery device of claim 17, wherein one or more linkers comprise Gly-Ser-Gly (“GSG”).

19. The drug delivery device of any one of claims 1 to 13, wherein one or more linkers comprise a polyether.

20. The drug delivery device of claim 19, wherein one or more linkers comprise polyethylene glycol.

21. The drug delivery device of any one of claims 1 to 20, wherein the one or more linkers are conjugated to the peptide and/or the drug via an ester, a carbonate, or a carbamate moiety.

22. The drug delivery device of any one of claims 1 to 21, wherein the one or more linkers comprise a succinimidyl thioether or a triazole moiety.

23. The drug delivery device of any one of claims 1 to 22, wherein the one or more drugs are hydrophobic.

24. The drug delivery device of any one of claims 1 to 23, wherein the one or more drugs are antiretrovirals, antimicrobial agents, antibacterial agents, antivirals, hormones, statins, β-blockers, ACE inhibitors, angiotensin receptor blockers, vitamins, steroids, biologics, anti-cancer drugs, allergy medications, anticoagulants, antiplatelet therapies, non-steroidal anti-inflammatory drugs, vaccines, or combinations thereof.

25. The drug delivery device of claim 24, wherein the one or more drugs are antiretrovirals.

26. The drug delivery device of claim 24, wherein the one or more drugs comprise zidovudine, cabotegravir, dapivirine, fluticasone propionate, chlordiazepoxide, haloperidol, indomethacin, prednisone, ethinyl estradiol, acyclovir, tenofovir, atenolol, aminoglycosides, exenatide acetate, leuprolide acetate, acetylsalicylic acid (aspirin), levodopa, remdesivir, acyclovir, ganciclovir, ribavirin, lamivudine, dolutegravir, chloroquine, hydroxychloroquine, azithromycin, lopinavir, ritonavir, EV-100, EV-200, EV-300, EV-400, KD025, tenofovir, emtricitabine, elvitegravir, lenacapavir, islatravir, C5A, VRC01, or combinations thereof.

27. The drug delivery device of claim 26, wherein the one or more drugs comprise tenofovir.

28. The drug delivery device of claim 27, wherein the one or more drugs comprise tenofovir alafenamide or tenofovir disoproxil fumarate.

29. The drug delivery device of any one of claims 1 to 28, wherein the drug delivery device is a subdermal or intramuscular implant, an intravaginal ring, or a microarray patch.

30. The drug delivery device of any one of claims 1 to 29, comprising one kernel.

31. The drug delivery device of any one of claims 1 to 29, comprising a plurality of kernels.

32. The drug delivery device of any one of claims 1 to 31, wherein the kernel or kernels comprise a defined microscopic or nanoscopic pore structure.

33. The drug delivery device of any one of claims 1 to 32, wherein the kernel is a reservoir kernel.

34. The drug delivery device of claim 33, wherein the reservoir kernel comprises a powder comprising one or more APIs.

35. The drug delivery device of claim 34, wherein the powder comprises a microscale or nanoscale drug carrier.

36. The drug delivery device of claim 35, wherein the drug carrier is a bead, capsule, microgel, nanocellulose, dendrimer, or diatom.

37. The drug delivery device of claim 33, wherein the reservoir kernel comprises a paste comprising one or more peptide-drug conjugates.

38. The drug delivery device of claim 37, wherein the paste comprises an oil excipient, an ionic liquid, a phase inversion system, or a gel.

39. The drug delivery device of claim 38, wherein the phase inversion system comprises a biodegradable polymer, a combination of phospholipids and medium-chain triglycerides, or lyotropic liquid crystals.

40. The drug delivery device of claim 38, wherein the gel is a stimulus-responsive gel or a self-healing gel.

41. The drug delivery device of any one of claims 1 to 32, wherein the kernel is a pellet, tablet, or a microtablet.

42. The drug delivery device of any one of claims 1 to 32, wherein the kernel comprises a fiber-based carrier.

43. The drug delivery device of claim 42, wherein the fiber-based carrier comprises an electrospun microfiber or nanofiber.

44. The drug delivery device of claim 43, wherein the electrospun nanofiber is a Janus microfiber or nanofiber.

45. The drug delivery device of any one of claims 42 to 44, wherein the fiber-based carrier comprises random or oriented fibers.

46. The drug delivery device of claim 45, wherein the fiber-based carrier comprises bundles, yarns, woven mats, or non-woven mats of fibers.

47. The drug delivery device of claim 42, wherein the fiber-based carrier comprises rotary jet spun, wet spun, or dry-jet spun fibers.

48. The drug delivery device of any one of claims 42 to 47, wherein the fiber-based carrier comprises glucose, sucrose, or a polymer material.

49. The drug delivery device of any one of claims 1 to 32, wherein the kernel comprises a porous sponge.

50. The drug delivery device of claim 49, wherein the porous sponge comprises silicone, a silica sol-gel material, xerogel, mesoporous silica, polymeric microsponge, polyurethane foam, nanosponge, or aerogel.

51. The drug delivery device of claim 49 or 50, wherein the porous sponge comprises a porogen.

52. The drug delivery device of claim 51, wherein the porogen comprises a fiber mat.

53. The drug delivery device of claim 52, wherein the fiber mat comprises glucose or sucrose.

54. The drug delivery device of claim 51, wherein the porogen comprises a peptide-drug conjugate.

55. The drug delivery device of any one of claims 49 to 54, wherein the porous sponge is impregnated with the peptide-drug conjugate.

56. The drug delivery device of claim 55, wherein the porous sponge comprises a sponge material that has an affinity for a solvent capable of dissolving a peptide-drug conjugate.

57. The drug delivery device of claim 56, wherein the porous sponge comprises polydimethylsiloxane (PDMS).

58. The drug delivery device of any one of claims 1 to 57, comprising one skin.

59. The drug delivery device of any one of claims 1 to 57, comprising a plurality of skins.

60. The drug delivery device of any one of claims 1 to 59, wherein the skin covers part of the device.

61. The drug delivery device of any one of claims 1 to 59, wherein the skin covers the entire device.

62. The drug delivery device of any one of claims 1 to 61, wherein the skin comprises a rate-limiting skin.

63. The drug delivery device of claim 62, wherein the skin is non-resorbable.

64. The drug delivery device of claim 63, wherein the skin comprises a biocompatible elastomer.

65. The drug delivery device of claim 64, wherein the skin comprises poly(dimethyl siloxane), silicone, one or more synthetic polymers, and/or metal.

66. The drug delivery device of claim 65, wherein the synthetic polymer is a poly(ether), poly(acrylate), poly(methacrylate), poly(vinyl pyrolidone), poly(vinyl acetate), poly(urethane), cellulose, cellulose acetate, poly(siloxane), poly(ethylene), poly(tetrafluoroethylene) and other fluorinated polymers, poly(siloxanes), copolymers thereof, or combinations thereof.

67. The drug delivery device of claim 66, wherein the polymer is expanded poly(tetrafluoroethylene) (ePTFE).

68. The drug delivery device of claim 6, wherein the polymer is ethylene vinyl acetate (EVA).

69. The drug delivery device of claim 65, wherein the metal is titanium, nickel-titanium (Nitinol) alloy, or stainless steel.

70. The drug delivery device of any one of claims 58 to 62, wherein the skin is resorbable.

71. The drug delivery device of claim 70, wherein the skin comprises a biocompatible elastomer.

72. The drug delivery device of claim 71, wherein the skin comprises poly(amides), poly(esters), poly(ester amides), poly(anhydrides), poly(orthoesters), polyphosphazenes, pseudo poly(amino acids), poly(glycerol-sebacate), poly(lactic acids), poly(glycolic acids), poly(lactic-co-glycolic acids), poly(caprolactones) (PCLs), PCL derivatives, amino alcohol-based poly(ester amides) (PEA), poly(octane-diol citrate) (POC), copolymers thereof, or mixtures thereof.

73. The drug delivery device of claim 72, wherein the polymer is crosslinked PCL.

74. The drug delivery device of claim 73, wherein the crosslinked PCL comprises lysine diisocyanate or 2,2-bis(-caprolacton-4-yl)propane.

75. The drug delivery device of claim 73, wherein the polymer comprises poly(caprolactone)/poly(lactic-co-glycolic acid) and tri-calcium phosphate.

76. The drug delivery device of any one of claims 1 to 75, wherein the skin is fabricated via casting and etching, soft lithography, or microlithography.

77. The drug delivery device of claim 76, wherein the skin is fabricated via microlithography.

78. The drug delivery device of claim 76 or 77, wherein the skin comprises a defined surface morphology.

79. The drug delivery device of claim 78, wherein the defined surface morphology comprises a grid pattern.

80. The drug delivery device of any one of claims 1 to 79, wherein the defined pores are microscopic or nanoscopic pores.

81. The drug delivery device of any one of claims 1 to 80, wherein the defined pores have a diameter less than 2 nm.

82. The drug delivery device of any one of claims 1 to 80, wherein the defined pores have a diameter of 2 nm to 50 nm.

83. The drug delivery device of any one of claims 1 to 80, wherein the defined pores have a diameter greater than 50 nm.

84. The drug delivery device of any one of claims 1 to 83 for implantation into the body of a patient.

85. The drug delivery device of claim 86, wherein implantation into the body comprises implantation into a sterile anatomic compartment.

86. The drug delivery device of claim 85, wherein the sterile anatomic compartment is selected from the subcutaneous space, the intramuscular space, the eye, the ear, and the brain.

87. The drug delivery device of claim 84, wherein implantation into the body comprises implantation into a nonsterile anatomic compartment.

88. The drug delivery device of claim 87, wherein the nonsterile anatomic compartment is selected from the vagina, the rectum, and the nasal cavity.

89. The drug delivery device of any one of claims 1 to 88, further comprising a shape adapted to be disposed within the body of a patient.

90. The drug delivery device of claim 89, wherein the device is capsule-shaped.

91. The drug delivery device of claim 89 or 90, wherein the device comprises one or more reservoirs.

92. The drug delivery device of claim 91, wherein the one or more reservoirs are separated into one or more compartments.

93. The drug delivery device of claim 92, wherein the one or more reservoirs are separated into compartments by one or more rib structures.

94. The drug delivery device of any one of claims 90 to 93, wherein the device further comprises one or more non-permeable disk-shaped covers.

95. The drug delivery device of claim 94, wherein the device comprises an outer sealing ring that forms a seal with the one or more covers.

96. The device of claim 95, wherein the one or more covers comprise an outer lip that fits inside the sealing ring to form a seal.

97. The drug delivery device of any one of claims 94 to 96, comprising one cover.

98. The drug delivery device of any one of claims 94 to 96, comprising two covers.

99. The drug delivery device of any one of claims 91 to 93, wherein the reservoir is sealed by the skin.

100. The drug delivery device of claim 99, wherein the skin is attached to the device with an adhesive.

101. The drug delivery device of any one of claims 92 to 100, wherein the one or more kernels are disposed within the one or more compartments.

102. The drug delivery device of claim 89, wherein the device is in the shape of a torus.

103. The drug delivery device of claim 102, comprising one or more cylindrical core elements disposed within a first skin, wherein the core elements comprise a kernel and optionally a second skin.

104. The drug delivery device of claim 102, comprising a molded lower structure comprising one or more compartments containing one or more kernels, and an upper structure bonded to the lower carrier to seal the plurality of compartments.

105. The drug delivery device of claim 104, wherein the skin covers the lower carrier.

106. The drug delivery device of claim 104, wherein the skin covers the lower structure and the upper structure.

107. The drug delivery device of any one of claims 102 to 106, comprising one or more lobes protruding inward from the outer edge of the torus.

108. The drug delivery device of claim 107, wherein the one or more compartments are disposed in the lobes.

109. The drug delivery device of claim 107 or 108, comprising one or more recessed structures to facilitate sealing of the device.

110. The drug delivery device of any one of claims 107 to 109, wherein the one or more compartments comprise ribs.

111. The drug delivery device of any one of claims 107 to 110, further comprising a protective mesh disposed over the surface of the device.

112. A method of delivering one or more APIs to a subject in need thereof, the method comprising providing the drug delivery device of any one of claims 1 to 111 to the subject.

113. The method of claim 112, wherein providing the drug delivery device comprises implanting the drug delivery device into the subject's body.

114. A method of treating or preventing a disease or disorder in a subject, the method comprising providing the drug delivery device of any one of claims 1 to 111 to the subject.

115. The method of claim 114, wherein the disease or disorder is an infectious disease, a transplant graft rejection, a condition indicating hormonal therapy, a physiological or pathophysiological application, diabetes mellitus, an allergy or hypersensitivity, an autoimmune disorder, cancer, a hematological disease, a musculoskeletal disorder, a psychological or neurological disorder, a genetic disease, or a veterinary condition.

116. The method of claim 115, wherein the disease or disorder is an infectious disease.

117. The method of claim 116, wherein the infectious disease is HIV.


SEQUENCE LISTING (119 sequences)

SEQ ID NO: 1 - protein, 12 aa, Synthetic construct
AHLPIVRASLPS

SEQ ID NO: 2 - protein, 17 aa, Synthetic construct
KGVSLSYRKKGVSLSYR

SEQ ID NO: 3 - protein, 8 aa, Synthetic construct
KGVSLSYR

SEQ ID NO: 4 - protein, 12 aa, Synthetic construct
YSAYPDSVPMMS

SEQ ID NO: 5 - protein, 12 aa, Synthetic construct
SWLAYPGAVSYR

SEQ ID NO: 6 - protein, 9 aa, Synthetic construct
CHVLWSTRC

SEQ ID NO: 7 - protein, 9 aa, Synthetic construct
CVSNPRWKC

SEQ ID NO: 8 - protein, 15 aa, Synthetic construct
QEYSPNLWGHEFRSH

SEQ ID NO: 9 - protein, 15 aa, Synthetic construct
QEFSPNLWGLEFQKN

SEQ ID NO: 10 - protein, 14 aa, Synthetic construct
QEFSPYMGLEFKKH

SEQ ID NO: 11 - protein, 6 aa, Synthetic construct
KCCYSL

SEQ ID NO: 12 - protein, 24 aa, Synthetic construct
AEGEFMYWGDSHWLQYWYEGDPAK

SEQ ID NO: 13 - protein, 6 aa, Synthetic construct
MSRTMS

SEQ ID NO: 14 - protein, 6 aa, Synthetic construct
MARAKE

SEQ ID NO: 15 - protein, 6 aa, Synthetic construct
MARSGL

SEQ ID NO: 16 - protein, 7 aa, Synthetic construct
NLYVASW

SEQ ID NO: 17 - protein, 7 aa, Synthetic construct
TLTVLPW

SEQ ID NO: 18 - protein, 7 aa, Synthetic construct
LTVSPWY

SEQ ID NO: 19 - protein, 18 aa, Synthetic construct
BCDSDSDITWDQLWDLMK

SEQ ID NO: 20 - protein, 15 aa, Synthetic construct
GSWYAWSPLVPSAQI

SEQ ID NO: 21 - protein, 15 aa, Synthetic construct
RFRGLISLSQVYLSP

SEQ ID NO: 22 - protein, 15 aa, Synthetic construct
HGRFILPWWYAFSPS

SEQ ID NO: 23 - protein, 16 aa, Synthetic construct
PRNICSRRDPTCWTTY

SEQ ID NO: 24 - protein, 16 aa, Synthetic construct
AYTKCSRQWRTCMTTH

SEQ ID NO: 25 - protein, 16 aa, Synthetic construct
LTGTCLQYQSRCGNTR

SEQ ID NO: 26 - protein, 15 aa, Synthetic construct
LYPLHTYTPLSLPLF

SEQ ID NO: 27 - protein, 15 aa, Synthetic construct
PKMTLQRSNIRPSMP

SEQ ID NO: 28 - protein, 15 aa, Synthetic construct
GVTDSSTSNLDMPHW

SEQ ID NO: 29 - protein, 15 aa, Synthetic construct
APRPGPWLWSNADSV

SEQ ID NO: 30 - protein, 15 aa, Synthetic construct
DKPTAFVSVYLKTAL

SEQ ID NO: 31 - protein, 15 aa, Synthetic construct
SMEPALPDWWWKMFK

SEQ ID NO: 32 - protein, 15 aa, Synthetic construct
PQNSKIPGPTFLDPH

SEQ ID NO: 33 - protein, 22 aa, Synthetic construct
AEGEFGWWGDSHWLQYEGDPAK

SEQ ID NO: 34 - protein, 24 aa, Synthetic construct
AEGEFGHWCDQHWLQYWYEGDPAK

SEQ ID NO: 35 - protein, 24 aa, Synthetic construct
AEGEFLMWGGSHWLEYWYEGDPAK

SEQ ID NO: 36 - protein, 24 aa, Synthetic construct
AEGEFPRWGDSHWLQYWYEGDPAK

SEQ ID NO: 37 - protein, 22 aa, Synthetic construct
AEGEFIHNRYNRFFYWYGDPAK

SEQ ID NO: 38 - protein, 22 aa, Synthetic construct
AEGEFWGDSHWLQYWYEGDPAK

SEQ ID NO: 39 - protein, 12 aa, Synthetic construct
SVSVGMKPSPRP

SEQ ID NO: 40 - protein, 7 aa, Synthetic construct
GNGRAHA

SEQ ID NO: 41 - protein, 9 aa, Synthetic construct
CRGDKGPDC

SEQ ID NO: 42 - protein, 6 aa, Synthetic construct
RGDGWK

SEQ ID NO: 43 - protein, 9 aa, Synthetic construct
CDCRGDCFC

SEQ ID NO: 44 - protein, 6 aa, Synthetic construct
CRGDCF

SEQ ID NO: 45 - skipped

SEQ ID NO: 46 - protein, 6 aa, Synthetic construct
GPLPLR

SEQ ID NO: 47 - protein, 5 aa, Synthetic construct
CRGDK

SEQ ID NO: 48 - protein, 32 aa, Synthetic construct
KDEPQRRSARLSAKPAPPKPEPKPKKAPAKKC

SEQ ID NO: 49 - protein, 31 aa, Synthetic construct
KDEPQRRSARLSAKPAPPKPEPKPKKAPAKK

SEQ ID NO: 50 - protein, 31 aa, Synthetic construct; identical to SEQ ID NO: 49

SEQ ID NO: 51 - protein, 9 aa, Synthetic construct
CLDGGRPKC

SEQ ID NO: 52 - protein, 9 aa, Synthetic construct
CLSDGKRKC

SEQ ID NO: 53 - protein, 10 aa, Synthetic construct
LTLRWVGLMS

SEQ ID NO: 54 - protein, 8 aa, Synthetic construct
KGVSLSYR

SEQ ID NO: 55 - protein, 19 aa, Synthetic construct
TFFYGGSRGKRNNFKTEEY

SEQ ID NO: 56 - protein, 10 aa, Synthetic construct
LRKLRKRLLR

SEQ ID NO: 57 - protein, 5 aa, Synthetic construct
CNGRC

SEQ ID NO: 58 - protein, 7 aa, Synthetic construct
  REGION 6: note = The entire sequence of amino acids 1-5 is repeated three times.
KKEEEXK

SEQ ID NO: 59 - protein, 6 aa, Synthetic construct
  SITE 6: note = X denotes L-NH2
WAVGHX

SEQ ID NO: 60 - skipped

SEQ ID NO: 61 - protein, 6 aa, Synthetic construct
VQIVYK

SEQ ID NO: 62 - protein, 12 aa, Synthetic construct
KYLAYPDSVHIW

SEQ ID NO: 63 - protein, 7 aa, Synthetic construct
SSKYQSL

SEQ ID NO: 64 - protein, 5 aa, Synthetic construct
  REGION 1..5: note = The peptide RGDFK is circular.
RGDFK

SEQ ID NO: 65 - skipped

SEQ ID NO: 66 - protein, 5 aa, Synthetic construct
QEXSP

SEQ ID NO: 67 - skipped

SEQ ID NO: 68 - protein, 5 aa, Synthetic construct
CSRQW

SEQ ID NO: 69 - protein, 5 aa, Synthetic construct
AEGEF

SEQ ID NO: 70 - skipped

SEQ ID NO: 71 - protein, 13 aa, Synthetic construct
GRKKRRQRRRPPQ

SEQ ID NO: 72 - protein, 12 aa, Synthetic construct
TATRKKRRQRRR

SEQ ID NO: 73 - protein, 17 aa, Synthetic construct
TRQARRNRRRRWRERQR

SEQ ID NO: 74 - protein, 15 aa, Synthetic construct
RRRRNRTRRNRRRVR

SEQ ID NO: 75 - protein, 16 aa, Synthetic construct
RQIKIWFQNRRMKWKK

SEQ ID NO: 76 - protein, 33 aa, Synthetic construct
GDCLPHLKLCKENKDCCSKKCKRRGTNIEKRCR

SEQ ID NO: 77 - protein, 27 aa, Synthetic construct
GWTLNSAGYLLGKINLKALAALAKKIL

SEQ ID NO: 78 - protein, 7 aa, Synthetic construct
PKKKRKV

SEQ ID NO: 79 - protein, 10 aa, Synthetic construct
VQRKRQKLMP

SEQ ID NO: 80 - protein, 8 aa, Synthetic construct
SKKKKTKV

SEQ ID NO: 81 - protein, 8 aa, Synthetic construct
GRKRKKRT

SEQ ID NO: 82 - protein, 8 aa, Synthetic construct
ERKKRRRE

SEQ ID NO: 83 - protein, 7 aa, Synthetic construct
FKKFRKF

SEQ ID NO: 84 - protein, 8 aa, Synthetic construct
RRMKWKKK

SEQ ID NO: 85 - protein, 4 aa, Synthetic construct
  REGION 1..4: note = The entire sequence of amino acids 1-4 can be repeated but not more than 12 peptides in total.
RRRR

SEQ ID NO: 86 - protein, 4 aa, Synthetic construct
  REGION 1..4: note = The entire sequence of amino acids 1-4 can be repeated but not more than 12 peptides in total.
KKKK

SEQ ID NO: 87 - protein, 4 aa, Synthetic construct
  REGION 1..4: note = The entire sequence of amino acids 1-4 can be repeated but not more than 12 peptides in total.
PPPP

SEQ ID NO: 88 - protein, 26 aa, Synthetic construct
GIGAVLKVLTTGLPALISWIKRKRQQ

SEQ ID NO: 89 - protein, 21 aa, Synthetic construct
GFCWYVCVYRNGVRVCYRRCN

SEQ ID NO: 90 - protein, 21 aa, Synthetic construct
KETWWETWWTEWSQPKKKRKV

SEQ ID NO: 91 - protein, 21 aa, Synthetic construct
KKTWWKTWWTKWSQPKKKRKV

SEQ ID NO: 92 - protein, 27 aa, Synthetic construct
GALFLGFLGAAGSTMGAWSQPKKKRKV

SEQ ID NO: 93 - protein, 20 aa, Synthetic construct
GLFRALLRLLRSLWRLLLRA

SEQ ID NO: 94 - protein, 20 aa, Synthetic construct
HGLASTLTRWAHYNALIRAF

SEQ ID NO: 95 - protein, 13 aa, Synthetic construct
PLIYLRLLRGQWC

SEQ ID NO: 96 - protein, 21 aa, Synthetic construct
AGYLLGKINLKALAALAKKIL

SEQ ID NO: 97 - protein, 23 aa, Synthetic construct
LIRLWSHLIHIWFQNRRLKWKKK

SEQ ID NO: 98 - protein, 22 aa, Synthetic construct
MVTVLFRRLRIRRACGPPRVRV

SEQ ID NO: 99 - protein, 22 aa, Synthetic construct
LGTYTQDFNKTFPQTAIGVGAP

SEQ ID NO: 100 - protein, 18 aa, Synthetic construct
RGGRLSYSRRRFSTSTGR

SEQ ID NO: 101 - protein, 18 aa, Synthetic construct
LLIILRRRIRKQAHAHSK

SEQ ID NO: 102 - protein, 16 aa, Synthetic construct
KLALKLALKALKAALK

SEQ ID NO: 103 - protein, 9 aa, Synthetic construct
KLAKLAKLA

SEQ ID NO: 104 - protein, 18 aa, Synthetic construct
VRLPPPVRLPPPVRLPPP

SEQ ID NO: 105 - protein, 20 aa, Synthetic construct
GLWRALWRLLRSLWRLLWRA

SEQ ID NO: 106 - protein, 17 aa, Synthetic construct
CSIPPEVKFNKPFVYLI

SEQ ID NO: 107 - protein, 6 aa, Synthetic construct
PFVYLI

SEQ ID NO: 108 - protein, 15 aa, Synthetic construct
SDLWEMMMVSLACQY

SEQ ID NO: 109 - protein, 12 aa, Synthetic construct
YTFGLKTSFNVQ

SEQ ID NO: 110 - protein, 15 aa, Synthetic construct
AAVALLPAVLLALLP

SEQ ID NO: 111 - protein, 34 aa, Synthetic construct
  REGION 1..34: note = The peptide GGVCPKILKKCRRDSDCPGACICRGNGYCGSGSD is circular.
GGVCPKILKKCRRDSDCPGACICRGNGYCGSGSD

SEQ ID NO: 112 - protein, 34 aa, Synthetic construct
  REGION 1..34: note = The peptide GGVCPKILAACRRDSDCPGACICRGNGYCGSGSD is circular.
GGVCPKILAACRRDSDCPGACICRGNGYCGSGSD

SEQ ID NO: 113 - protein, 34 aa, Synthetic construct
  REGION 1..34: note = The peptide GGVCPAILKKCRRDSDCPGACICRGNGYCGSGSD is circular.
GGVCPAILKKCRRDSDCPGACICRGNGYCGSGSD

SEQ ID NO: 114 - protein, 34 aa, Synthetic construct
  REGION 1..34: note = The peptide GGVCPKILAKCRRDSDCPGACICRGNGYCGSGSD is circular.
GGVCPKILAKCRRDSDCPGACICRGNGYCGSGSD

SEQ ID NO: 115 - protein, 34 aa, Synthetic construct
  REGION 1..34: note = The peptide GGVCPKILKACRRDSDCPGACICRGNGYCGSGSD is circular.
GGVCPKILKACRRDSDCPGACICRGNGYCGSGSD

SEQ ID NO: 116 - protein, 29 aa, Synthetic construct
  REGION 1..29: note = The peptide GLPVCGETCVGGTCNTPGCTCSWPVCTRN is circular.
GLPVCGETCVGGTCNTPGCTCSWPVCTRN

SEQ ID NO: 117 - protein, 29 aa, Synthetic construct
  REGION 1..29: note = The peptide GLPVCGETCVGGTCNTPGCKCSWPVCTRN is circular.
GLPVCGETCVGGTCNTPGCKCSWPVCTRN

SEQ ID NO: 118 - protein, 29 aa, Synthetic construct
  REGION 1..29: note = The peptide GLPVCGETCVGGTCNTPGCTCSWPKCTRN is circular.
GLPVCGETCVGGTCNTPGCTCSWPKCTRN

SEQ ID NO: 119 - protein, 14 aa, Synthetic construct
  REGION 1..14: note = The peptide GRCTKSIPPICFPD is circular.
GRCTKSIPPICFPD